(19) United States
(12) Patent Application Publication
(10) Pub. No.: US 2010/0144717 A1
(43) Pub. Date: Jun. 10, 2010

(54) 2-QUINOLINONE AND 2-QUINOXALINONE-DERIVATIVES AND THEIR USE AS ANTIBACTERIAL AGENTS

(76) Inventors: Janelle Comita-Prevoir, Waltham, MA (US); Mark Cronin, Waltham, MA (US); Bolin Geng, Waltham, MA (US); Andrew Aydon Godfrey, Cheshire (GB); Folkert Reck, Waltham, MA (US)

Correspondence Address: ASTRAZENECA R&D BOSTON, 35 GATEHOUSE DRIVE, WALTHAM, MA, 02451-1215, US

(21) Appl. No.: 12/519,338

(22) PCT Filed: Dec. 15, 2006
(86) PCT No.: PCT/GB2007/004766

§ 371 Date: Dec. 17, 2009

Related U.S. Application Data
(60) Provisional application No. 60/870,181, filed on Dec. 15, 2006.

(51) Int. Cl. A61K 31/542; C07D 491/056; A61K 31/4709; C07D 498/04; A61K 31/5383; C07D 513/04; A61K 31/498; A61P 31/04

(52) U.S. Cl. 514/224.2; 546/115; 514/302; 544/105; 514/230.5; 544/48; 544/354; 514/249

(57) ABSTRACT

The present invention relates to compounds of Formula (I): and pharmaceutically acceptable salts thereof, to their use in the treatment of bacterial infections, and to their methods of preparation.

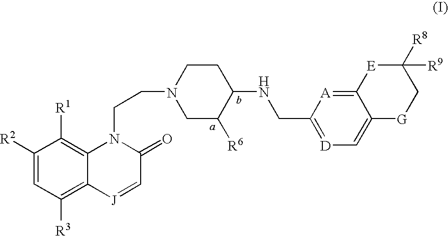

### FIELD OF INVENTION

The present invention relates to novel piperidines, pharmaceutical compositions thereof, and methods of use. In addition, the present invention relates to therapeutic methods for the treatment of bacterial infections.

### BACKGROUND

The international health community continues to express serious concern that the evolution of antibacterial resistance will result in strains against which currently available antibacterial agents will be ineffective. For example, resistant strains of Gram-positive pathogens such as methicillin-resistant Staphylococcus aureus (MRSA), methicillin-resistant coagulase-negative staphylococci (MRCNS), penicillin-resistant Streptococcus pneumoniae and multiple resistant Enterococcus faecium are both difficult to treat and difficult to eradicate. Consequently, in order to overcome the threat of widespread multi-drug resistant organisms, there is an on-going need to develop new antibiotics, particularly those with either a novel mechanism of action and/or containing new pharmacophoric groups.

### SUMMARY

In accordance with the present invention, the applicants have hereby discovered compounds that possess the ability to act as antimicrobials. Accordingly, the present invention relates to compounds that demonstrate antibacterial activity, processes for their preparation, pharmaceutical compositions containing them as the active ingredient, their use as medicaments, and their use in the manufacture of medicaments for use in the treatment of bacterial infections in warm-blooded animals such as humans.

Accordingly the present invention provides a compound of Formula (I):

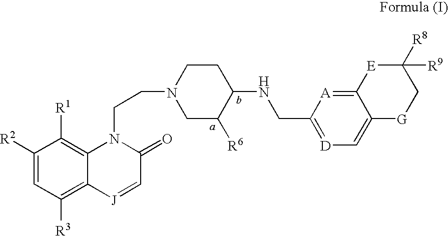

or a pharmaceutically acceptable salt thereof, wherein the compound of Formula (I) is substantially free of a cis (±) mixture of its enantiomers, and wherein
A is selected from CH and N;
D is selected from C—R⁷ and N;

wherein at least one of A and D is carbon;
E is selected from O, NH, and S,
wherein:

i) E is NH if R⁸ and R⁹ together from ═O; and

ii) E is O or S if R⁸ and R⁹ are each H;
G is selected from O and S;
J is selected from C—R⁴ and N;
R¹ is selected from H, halo, cyano, C₁₋₆alkyl, C₂₋₆alkenyl, and C₂₋₆alkynyl, OR^{1a}, and —N(R^{1a})₂, wherein said C₁₋₆alkyl, C₂₋₆alkenyl, and C₂₋₆alkynyl are optionally substituted with one or more R¹⁰;
R^{1a} in each occurrence is independently selected from H and C₁₋₆alkyl, wherein said C₁₋₆alkyl is optionally substituted with one or more R²⁰;
R² is selected from H, halo, cyano, C₁₋₆alkyl, C₂₋₆alkenyl, C₂₋₆alkynyl, —OR^{2a}, and —N(R^{2a})₂ wherein said C₁₋₆alkyl, C₂₋₆alkenyl, and C₂₋₆alkynyl are optionally substituted with one or more R²⁰;
R^{2a} in each occurrence is independently selected from H and C₁₋₆alkyl, wherein said C₁₋₆alkyl are optionally substituted with one or more R²⁰;
R³ is selected from H, halo, cyano, C₁₋₆alkyl, C₂₋₆alkenyl, and C₂₋₆alkynyl, —OR^{3a}, and —N(R^{3a})₂, wherein said C₁₋₆alkyl, C₂₋₆alkenyl, and C₂₋₆alkynyl are optionally substituted with one or more R³⁰;
R^{3a} in each occurrence is independently selected from H and C₁₋₆alkyl, wherein said C₁₋₆alkyl is optionally substituted with one or more R³⁰;
R⁴ is selected from H, halo, —CO₂H, cyano, C₁₋₆alkyl, C₂₋₆alkenyl, and C₂₋₆alkynyl, wherein said C₁₋₆alkyl, C₂₋₆alkenyl, and C₂₋₆alkynyl are optionally substituted with one or more R⁴⁰;
R⁶ is selected from fluoro, C₁₋₆alkyl, C₂₋₆alkenyl, C₂₋₆alkynyl, —OR^{6a}, wherein said C₁₋₆alkyl, C₂₋₆alkenyl, and C₂₋₆alkynyl are optionally substituted with one or more R⁶⁰;
R^{6a} in each occurrence is independently selected from H and C₁₋₆alkyl, wherein said C₁₋₆alkyl are optionally substituted with one or more R⁶⁰;
R⁷ is selected from H, halo, cyano, C₁₋₆alkyl, C₂₋₆alkenyl, and C₂₋₆alkynyl, wherein said C₁₋₆alkyl, C₂₋₆alkenyl, and C₂₋₆alkynyl are optionally substituted with one or more R⁷⁰;
R⁸ and R⁹ are each hydrogen, or R⁸ and R⁹ together form ═O; and
R¹⁰, R²⁰, R³⁰, R³⁰, R⁶⁰, and R⁷⁰ in each occurrence are each, independently, selected from halo, hydroxy, cyano, —CO₂H, C₁₋₆alkyl, C₂₋₆alkenyl, and C₂₋₆alkynyl.

In another aspect, the present invention provides a compound of Formula (II):

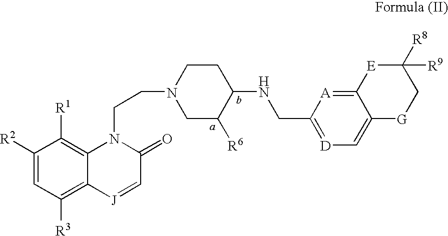

or a pharmaceutically acceptable salt thereof, wherein the R⁶ group on carbon “a” and the —NH— group on carbon “b” are in a trans relationship to each other, and wherein
A is selected from CH and N;
D is selected from C—R⁷ and N;

wherein at least one of A and D is carbon;
E is selected from O, NH, and S,
wherein:

i) E is NH if R⁸ and R⁹ together from ═O; and
ii) E is O or S if R⁸ and R⁹ are each H;
G is selected from O and S;
J is selected from C—R⁴ and N;
R¹ is selected from H, halo, cyano, C₁₋₆alkyl, C₂₋₆alkenyl, and C₂₋₆alkynyl, OR^{1a}, and —N(R^{1a})₂, wherein said C₁₋₆alkyl, C₂₋₆alkenyl, and C₂₋₆alkynyl are optionally substituted with one or more R¹⁰;
R^{1a} in each occurrence is independently selected from H and C₁₋₆alkyl, wherein said C₁₋₆alkyl is optionally substituted with one or more R²⁰;
R² is selected from H, halo, cyano, C₁₋₆alkyl, C₂₋₆alkenyl, C₂₋₆alkynyl, —OR^{2a}, and —N(R^{2a})₂ wherein said C₁₋₆alkyl, C₂₋₆alkenyl, and C₂₋₆alkynyl are optionally substituted with one or more R²⁰;
R^{2a} in each occurrence is independently selected from H and C₁₋₆alkyl, wherein said C₁₋₆alkyl are optionally substituted with one or more R²⁰;
R³ is selected from H, halo, cyano, C₁₋₆alkyl, C₂₋₆alkenyl, and C₂₋₆alkynyl, —OR^{3a}, and —N(R^{3a})₂, wherein said C₁₋₆alkyl, C₂₋₆alkenyl, and C₂₋₆alkynyl are optionally substituted with one or more R³⁰;
R^{3a} in each occurrence is independently selected from H and C₁₋₆alkyl, wherein said C₁₋₆alkyl is optionally substituted with one or more R³⁰;
R⁴ is selected from H, halo, —CO₂H, cyano, C₁₋₆alkyl, C₂₋₆alkenyl, and C₂₋₆alkynyl, wherein said C₁₋₆alkyl, C₂₋₆alkenyl, and C₂₋₆alkynyl are optionally substituted with one or more R⁴⁰;
R⁶ is selected from fluoro, C₁₋₆alkyl, C₂₋₆alkenyl, C₂₋₆alkynyl, —OR^{6a}, wherein said C₁₋₆alkyl, C₂₋₆alkenyl, and C₂₋₆alkynyl are optionally substituted with one or more R⁶⁰;
R^{6a} in each occurrence is independently selected from H and C₁₋₆alkyl, wherein said C₁₋₆alkyl are optionally substituted with one or more R⁶⁰;
R⁷ is selected from H, halo, cyano, C₁₋₆alkyl, C₂₋₆alkenyl, and C₂₋₆alkynyl, wherein said C₁₋₆alkyl, C₂₋₆alkenyl, and C₂₋₆alkynyl are optionally substituted with one or more R⁷⁰;
R⁸ and R⁹ are each hydrogen, or R⁸ and R⁹ together form ═O; and
R¹⁰, R²⁰, R³⁰, R⁴⁰, R⁶⁰, and R⁷⁰ in each occurrence are each, independently, selected from halo, hydroxy, cyano, —CO₂H, C₁₋₆alkyl, C₂₋₆alkenyl, and C₂₋₆alkynyl.

The compounds of Formulas (I) and (II) and pharmaceutically acceptable salts thereof are believed to be effective in treating bacterial infections. Accordingly, the present invention relates to compounds of Formulas (I) and (II) that demonstrate antibacterial activity, processes for their preparation, pharmaceutical compositions containing them as the active ingredient, methods for their us in the treatment of bacterial infections, their use as medicaments for the treatment of bacterial infections, and their use in the manufacture of such medicaments.

### DETAILED DESCRIPTION OF THE INVENTION

In this specification the prefix C_{x-y} as used in terms such as C_{x-y}alkyl and the like (where x and y are integers) indicates the numerical range of carbon atoms that are present in the group; for example, C₁₋₄alkyl includes C₁alkyl (methyl), C₂alkyl (ethyl), C₃alkyl(propyl and isopropyl) and C₄alkyl(butyl, 1-methylpropyl, 2-methylpropyl, and t-butyl).

As used herein the term “alkyl” refers to both straight and branched chain saturated hydrocarbon radicals having the specified number of carbon atoms. References to individual alkyl groups such as “propyl” are specific for the straight chain version only and references to individual branched chain alkyl groups such as ‘isopropyl’ are specific for the branched chain version only.

The term “alkenyl” refers to both straight and branched chain hydrocarbon radicals having the specified number of carbon atoms and containing at least one carbon-carbon double bond. For example, “C₂₋₈alkenyl” includes, but is not limited to, groups such as C₂₋₆alkenyl, C₂₋₄alkenyl, ethenyl, 2-propenyl, 2-methyl-2-propenyl, 3-butenyl, 4-pentenyl, 5-hexenyl, 2-heptenyl, and 2-methyl-1-heptenyl.

The term “alkynyl” refers to both straight and branched chain hydrocarbon radicals having the specified number of carbon atoms and containing at least one carbon-carbon triple bond. For example, “C₂₋₈alkynyl” includes, but is not limited to, groups such as C₂₋₆alkynyl, C₂₋₄alkynyl, ethynyl, 2-propynyl, 2-methyl-2-propynyl, 3-butynyl, 4-pentynyl, 5-hexynyl, 2-heptynyl, and 4-methyl-5-heptynyl.

The term “halo” refers to fluoro, chloro, and bromo. In one aspect, “halo” may refer to fluoro.

Where optional substituents are chosen from “one or more” groups it is to be understood that this definition includes all substituents being chosen from one of the specified groups or the substituents being chosen from two or more of the specified groups.

The term “carbocyclyl” refers to a saturated, partially saturated, or unsaturated, mono or bicyclic carbon ring that contains 3-12 ring atoms, wherein one or more —CH₂— groups can optionally be replaced by a corresponding number of —C(O)— groups. In one aspect, the term “carbocyclyl” may refer to a monocyclic ring containing 3 to 6 ring atoms or a bicyclic ring containing 9 or 10 atoms. In another aspect, the term “carbocyclyl” may refer to a monocyclic ring containing 5 or 6 atoms. Illustrative examples of “carbocyclyl” include, but are not limited to, adamantyl, cyclopropyl, cyclobutyl, cyclopentyl, cyclopentenyl, cyclohexyl, cyclohexenyl, 1-oxocyclopentyl, phenyl, naphthyl, tetralinyl, indanyl or 1-oxoindanyl. A particular example of a “carbocyclyl” group is phenyl.

The term “heterocyclyl” refers to a saturated, partially saturated or unsaturated, mono or bicyclic ring containing 4-12 ring atoms of which at least one ring atom is selected from nitrogen, sulfur, and oxygen, and which may, unless otherwise specified, be carbon or nitrogen linked, wherein a —CH₂— group can optionally be replaced by a —C(O)—. Ring sulfur atoms may be optionally oxidized to form S-oxides. Ring nitrogen atoms may be optionally oxidized to form N-oxides. Illustrative examples of the term “heterocyclyl” include, but are not limited to, 1,3-benzodioxolyl, 3,5-dioxopiperidinyl, imidazolyl, indolyl, isoquinolone, isothiazolyl, isoxazolyl, morpholino, 2-oxopyrrolidinyl, 2-oxo-1,3-thiazolidinyl, piperazinyl, piperidyl, pyranyl, pyrazolyl, pyridinyl, pyrrolyl, pyrrolidinyl, pyrrolinyl, pyrimidyl, pyrazinyl, pyrazolyl, pyridazinyl, 4-pyridone, quinolyl, tetrahydropyranyl, thiazolyl, thiadiazolyl, thiazolidinyl, thienyl, thiomorpholino, thiophenyl, pyridine-N-oxide and quinoline-N-oxide. In one aspect of the invention the term “heterocyclyl” may refer to a saturated, partially saturated or unsaturated, monocyclic ring containing 5 or 6 atoms of which at least one atom is selected from nitrogen, sulfur, and oxygen, and may, unless otherwise specified, be carbon or nitrogen linked, and a ring nitrogen atom may be optionally oxidized to form an N-oxide.

The symbol “(±)” is intended to signify a racemic mixture; i.e. an optically inactive mixture of equal amounts of (+) and (−) enantiomers of the indicated compound. When a compound or mixture is designated as being “cis (±)” or “trans (±),” it should be understood that the cis or trans relationship indicated therein pertains to the relationship between the group on carbon “a” and the —NH— group on carbon “b.”

Where a particular R group is present in a compound of Formulas (I) or (II) more than once, it is intended that each selection for that R group is independent at each occurrence.

Unless specifically stated, the bonding atom of a group may be any suitable atom of that group; for example, propyl includes prop-1-yl and prop-2-yl.

The term “substantially free” is intended to indicate that the specified entity is present in an amount less than 10%, more particularly less than 5%, in particular less than 2%, more particularly less than 1%, in particular less then 0.5%, particularly less than 0.2%.

As used herein, the term “optionally substituted,” indicates that substitution is optional and therefore it is possible for the designated group to be either substituted or unsubstituted. In the event a substitution is desired, any number of hydrogens on the designated group may be replaced with a selection from the indicated substituents, provided that the normal valency of the atoms on a particular substituent is not exceeded, and that the substitution results in a stable compound which exhibits an antibacterial effect.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “effective amount” means an amount of a compound or composition which is sufficient to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician.

Compounds and substituent definitions of the present invention have been named with the aid of ACD/Name by ACD/Labs®.

Compounds of Formulas (I) and (II) and pharmaceutically acceptable salts thereof may form stable pharmaceutically acceptable acid or base salts, and in such cases administration of a compound as a salt may be appropriate, and pharmaceutically acceptable salts may be made by conventional methods well-known in the art.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The compounds of Formulas (I) and (II) have two chiral carbons on the central piperidine ring, carbon “a” and carbon “b.” Additionally, the compounds of Formulas (I) and (II) may have other chiral centers and/or geometric isomeric centers (E- and Z-isomers) It is to be understood that the invention encompasses all such optical, diastereoisomers and geometric isomers that possess antibacterial activity. The invention further relates to any and all tautomeric forms of the compounds of Formula (I) and (II) and pharmaceutically acceptable salts thereof that possess antibacterial activity.

It is also to be understood that certain compounds of Formulas (I) and (II) can exist in solvated as well as unsolvated forms such as, for example, hydrated forms. It is to be understood that the invention encompasses all such solvated forms which possess antibacterial activity.

Additional embodiments of the invention are as follows. Unless otherwise indicated, these additional embodiments relate to both compounds of Formulas (I) and (II), and to pharmaceutically acceptable salts thereof. The specific substituents and stereochemical relationships may be used, where appropriate, with any of the definitions, claims or embodiments defined hereinbefore or hereinafter.

Carbon “a” and Carbon “b”

In one aspect, the R⁶ group on carbon “a” and the —NH— group on carbon “b” of the compounds of Formula (I) are in a cis (+) relationship to each other, wherein the compound of Formula (I) is substantially free of a cis (±) mixture of its enantiomers.

In another aspect, the R⁶ group on carbon “a” and the —NH— group on carbon “b” of the compounds of Formula (I) are in a cis (−) relationship to each other, the compound of Formula (I) is substantially free of a cis (±) mixture of its enantiomers.

In still another aspect, the R⁶ group on carbon “a” and the —NH— group on carbon “b” of the compounds of Formula (I) are in a trans (+) relationship to each other.

In still another aspect, the R⁶ group on carbon “a” and the —NH— group on carbon “b” of the compounds of Formula (I) are in a trans (−) relationship to each other.

### A

In one aspect, A is N.

In another aspect, A is CH.

### D

In one aspect, D is N.

In another aspect, D is CH.

In still another aspect, D is selected from N and CH.

### A and D

In one aspect, A is N;

and D is CH.

In another aspect, A is CH; and

D is N.

### E, G, R⁸, and R⁹

In one aspect, E and G are each O; and

R⁸ and R⁹ are each H.

In another aspect, E is NH; G is selected from O and S; and

R⁸ and R⁹ together form ═O;

In still another aspect, E is NH; G is S; and

R⁸ and R⁹ together form ═O.

In yet another aspect, E is NH; G is O; and

R⁸ and R⁹ together form ═O.

### J

In one aspect, J is N.

In another aspect, J is CH.

In still another aspect, J is selected from N and CH.

In yet another aspect, J is selected from N and C—R⁴; and

R⁴ is selected from H and C₁₋₆alkyl.

In a further aspect, J is selected from N and C—R⁴; and

R⁴ is selected from H and methyl.

In still a further aspect, J is selected from C—R⁴; and

R⁴ is selected from H and methyl.

### A, D, E, G, R⁸, and R⁹

In one aspect, A, D, E, G, R⁸, and R⁹, together with the ring atoms to which they are attached, form a group selected from:

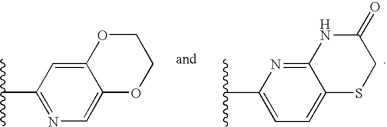

In another aspect, A, D, E, G, R⁸, and R⁹, together with the ring atoms to which they are attached, form:

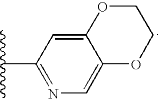

In still another aspect, A, D, E, G, R⁸, and R⁹, together with the ring atoms to which they are attached, form:

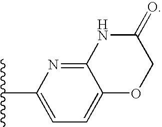

In yet another aspect, A, D, E, G, R⁸, and R⁹, together with the ring atoms to which they are attached, form:

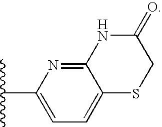

In a further aspect, A, D, E, G, R⁸, and R⁹, together with the ring atoms to which they are attached, form a group selected from:

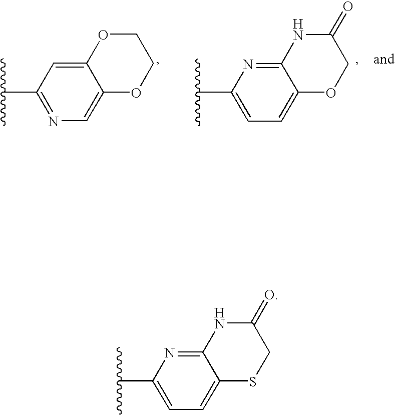

### R¹

In one aspect, R¹ is H.

### R²

In one aspect, R² is selected from cyano and —OR^{2a}; and

R^{2a} is C₁₋₆alkyl.

In another aspect, R² is selected from cyano and methoxy.

In still another aspect, R² is cyano.

In yet another aspect, R² is —OR^{2a}; and

R^{2a} is C₁₋₆alkyl.

In a further aspect, R² is methoxy.

### R³

In one aspect, R³ is H.

### R⁶

In one aspect, R⁶ is selected from fluoro and —OR^{6a}; and

R^{6a} is selected from H and C₁₋₆alkyl.

In another aspect, R⁶ is selected from fluoro, hydroxy, and methoxy.

In still another aspect, R⁶ is fluoro.

In yet another aspect, R⁶ is hydroxy.

In a further aspect, R⁶ is methoxy.

### R¹, R², R³, R⁶, R⁸, R⁹, A, D, E, G, and J

### Formula (I)

1) In one aspect, the compound of Formula (I), or a pharmaceutically acceptable salt thereof, may be a compound of Formula (Ia):

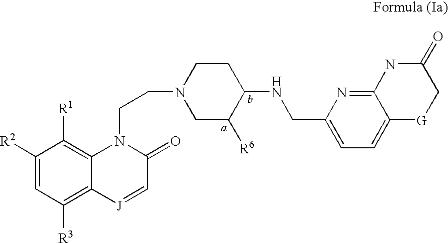

or a pharmaceutically acceptable salt thereof, wherein R¹, R², R³, R⁶, G, and J are as defined hereinabove, and wherein the compound of Formula (Ia) is substantially free of a cis (+) mixture of its enantiomers.
1.1) In one aspect of the compound of Formula (Ia), or a pharmaceutically acceptable salt thereof,

G is selected from O and S;

J is selected from N and CH;

R¹ is H;

R² is selected from cyano and —OR^{2a};

R^{2a} is C₁₋₆alkyl;

R³ is H;

R⁶ is selected from fluoro and —OR^{6a}; and

R^{6a} is selected from H and C₁₋₆alkyl.
1.2) In another aspect of the compound of Formula (Ia), or a pharmaceutically acceptable salt thereof,

G is selected from O and S;

J is selected from N and CH;

R¹ is H;

R² is selected from cyano and methoxy;

R³ is H; and

R⁶ is selected from fluoro, hydroxy, and methoxy.
1.3) In still another aspect of the compound of Formula (Ia), or a pharmaceutically acceptable salt thereof,

G is selected from O and S;

J is selected from N and C—R⁴;

R¹ is H;

R² is selected from cyano and —OR^{2a};

R^{2a} s C₁₋₆alkyl;

R³ is H;

R⁴ is selected from H and C₁₋₆alkyl;

R⁶ is selected from fluoro and —OR^{6a}; and

R^{6a} is selected from H and C₁₋₆alkyl.
1.4) In yet another aspect of the compound of Formula (Ia), or a pharmaceutically acceptable salt thereof,

G is selected from O and S;

J is selected from N and C—R⁴;

R¹ is H;

R² is selected from cyano and —OR^{2a};

R^{2a} is methyl;

R³ is H;

R⁴ is selected from H and methyl;

R⁶ is selected from fluoro and —OR^{6a}; and

R^{6a} is selected from H and methyl.
2) In another aspect, the compound of Formula (I), or a pharmaceutically acceptable salt thereof, is a compound of Formula (Ib):

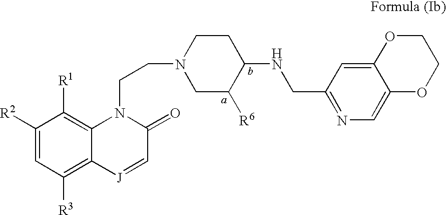

or a pharmaceutically acceptable salt thereof, wherein R¹, R², R³, R⁶, and J are as defined hereinabove, wherein the compound of Formula (Ib) is substantially free of a cis (±) mixture of its enantiomers.
2.1) In one aspect of the compound of Formula (Ib),

J is selected from N and CH;

R¹ is H;

R² is selected from cyano and —OR^{2a};

R^{2a} is C₁₋₆alkyl;

R³ is H;

R⁶ is selected from fluoro and —OR^{6a}; and

R^{6a} is selected from H and C₁₋₆alkyl.
2.2) In another aspect of the compound of Formula (Ib),

J is selected from N and CH;

R¹ is H;

R² is selected from cyano and methoxy;

R³ is H; and

R⁶ is selected from fluoro, hydroxy, and methoxy.
2.3) In still another aspect of the compound of Formula (Ib), or a pharmaceutically acceptable salt thereof,

J is selected from N and CH;

R¹ is H;

R² is selected from cyano and —OR^{2a};

R^{2a} is C₁₋₆alkyl;

R³ is H;

R⁶ is selected from fluoro and —OR^{6a}; and

R^{6a} is selected from H and C₁₋₆alkyl.
2.4) In yet another aspect of the compound of Formula (Ib), or a pharmaceutically acceptable salt thereof,

J is selected from N and CH;

R¹ is H;

R² is selected from cyano and methoxy;

R³ is H; and

R⁶ is selected from fluoro, hydroxy, and methoxy.
3) In still another aspect, the compound of Formula (I), or a pharmaceutically acceptable salt thereof, may be a compound of Formula (Ic):

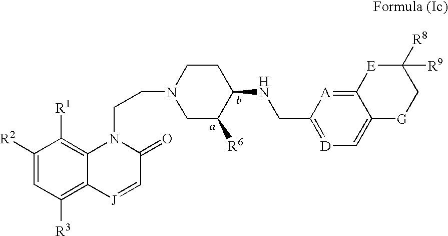

or a pharmaceutically acceptable salt thereof, wherein R¹, R², R³, R⁶, R⁸, R⁹, A, D, E,

G, and J are as defined hereinabove, wherein the compound of Formula (Ic) is substantially free of a cis (±) mixture of its enantiomers.
3.1) In one aspect of the compound of Formula (Ic), or a pharmaceutically acceptable salt thereof,

A, D, E, G, R⁸, and R⁹, together with the ring atoms to which they are attached form, a group selected from:

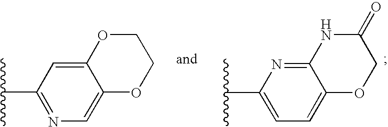

J is selected from N and CR⁴;

R¹ is H;

R² is selected from cyano and —OR^{2a};

R^{2a} is C₁₋₆alkyl;

R³ is H;

R⁴ is selected from H and C₁₋₆alkyl;

R⁶ is selected from fluoro and —OR^{6a}; and

R^{6a} is selected from H and C₁₋₆alkyl.
3.2) In another aspect of the compound of Formula (Ic), or a pharmaceutically acceptable salt thereof, A, D, E, G, R⁸, and R⁹, together with the ring atoms to which they are attached, form

A, D, E, G, R⁸, and R⁹, together with the ring atoms to which they are attached, form

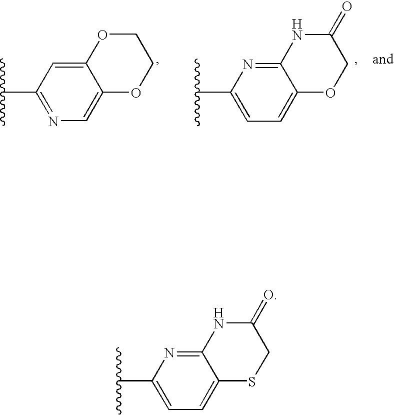

J is selected from N and CR⁴; R^{I} is H;

R¹ is H;

R^{2a} is C₁₋₆alkyl;

R³ is H;

R⁴ is selected from H and C₁₋₆alkyl;

R⁶ is selected from fluoro and —OR^{6a}; and

R^{6a} is selected from H and C₁₋₆alkyl.
3.3) In still another aspect of the compound of Formula (Ic), or a pharmaceutically acceptable salt thereof,

A, D, E, G, R⁸, and R⁹, together with the ring atoms to which they are attached, form a group selected from:

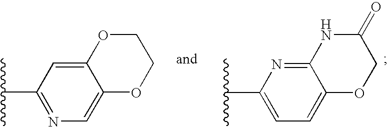

J is selected from N and CR″;

R¹ is H;

R² is H;

R^{2a} is methyl;

R³ is H;

R⁴ is selected from H and methyl;

R⁶ is selected from fluoro and —OR^{6a}; and

R^{6a} is selected from H and methyl.
3.4) In yet another aspect of the compound of Formula (Ic), or a pharmaceutically acceptable salt thereof,

A, D, E, G, R⁸, and R⁹, together with the ring atoms to which they are attached, form a group selected from:

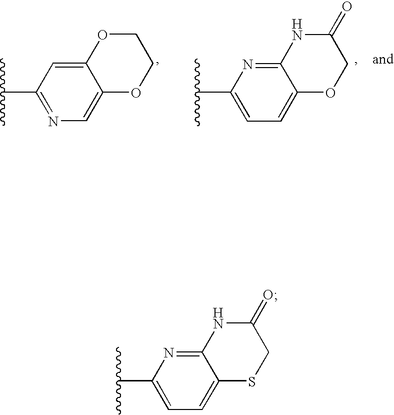

J is selected from N and CR⁴;

R¹ is H;

R² is H;

R^{2a} is methyl;

R³ is H;

R⁴ is selected from H and methyl;

R⁶ is selected from fluoro and —OR^{6a}; and

R^{6a} is selected from H and methyl.
4) In a further aspect, the compound of Formula (I), or a pharmaceutically acceptable salt thereof, may be a compound of Formula (Id):

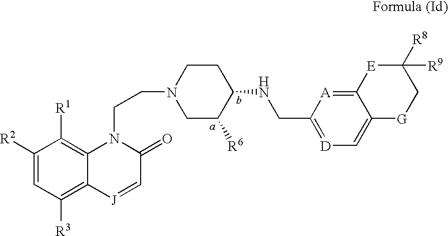

or a pharmaceutically acceptable salt thereof, wherein R¹, R², R³, R⁶, R⁸, R⁹, A, D, E, G, and J are as defined hereinabove, and wherein the compound of Formula (Id) is substantially free of a cis (±) mixture of its enantiomers.
4.1) In one aspect of the compound of Formula (Id), or a pharmaceutically acceptable salt thereof,

A, D, E, G, R⁸, and R⁹, together with the ring atoms to which they are attached, form a group selected from:

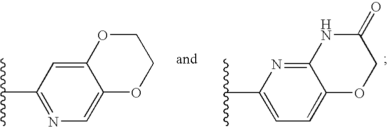

J is selected from N and CR⁴;

R¹ is H;

R² is H;

R^{2a} is C₁₋₆alkyl;

R³ is H;

R⁴ is selected from H and C₁₋₆alkyl;

R⁶ is selected from fluoro and —OR^{6a}; and

R^{6a} is selected from H and C₁₋₆alkyl.
4.2) In another aspect of the compound of Formula (Id), or a pharmaceutically acceptable salt thereof,

A, D, E, G, R⁸, and R⁹, together with the ring atoms to which they are attached, form a group selected from:

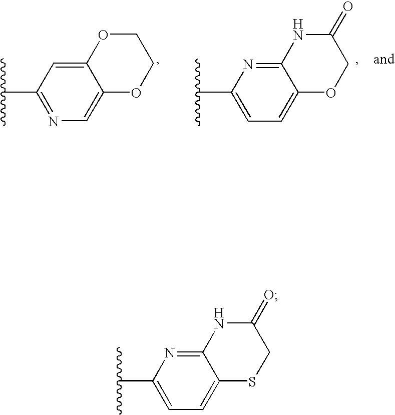

J is selected from N and CR⁴;

R¹ is H;

R² is H;

R^{2a} is C₁₋₆alkyl;

R³ is H;

R⁴ is selected from H and C₁₋₆alkyl;

R⁶ is selected from fluoro and —OR^{6a}; and

R^{6a} is selected from H and C₁₋₆alkyl.
4.3) In still another aspect of the compound of Formula (Id), or a pharmaceutically acceptable salt thereof,

A, D, E, G, R⁸, and R⁹, together with the ring atoms to which they are attached, form a group selected from:

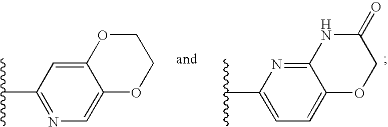

J is selected from N and CR⁴;

R¹ is H;

R² is selected from cyano and —OR^{2a};

R^{2a} is methyl;

R³ is H;

R⁴ is selected from H and methyl;

R⁶ is selected from fluoro and —OR^{6a}; and

R^{6a} is selected from H and methyl.
4.4) In yet another aspect of the compound of Formula (Id), or a pharmaceutically acceptable salt thereof,

A, D, E, G, R⁸, and R⁹, together with the ring atoms to which they are attached, form a group selected from:

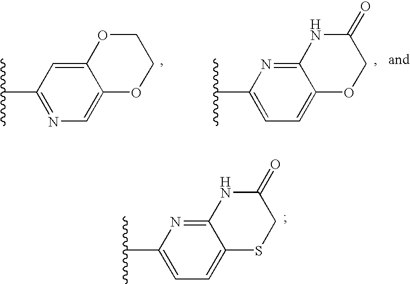

J is selected from N and CR⁴;

R¹ is H;

R² is selected from cyano and —OR^{2a};

R^{2a} is methyl;

R³ is H;

R⁴ is selected from H and methyl;

R⁶ is selected from fluoro and —OR^{6a}; and

R^{6a} is selected from H and methyl.
5) In still a further aspect, the compound of Formula (I), or a pharmaceutically acceptable salt thereof, may be a compound of Formula (Ie):

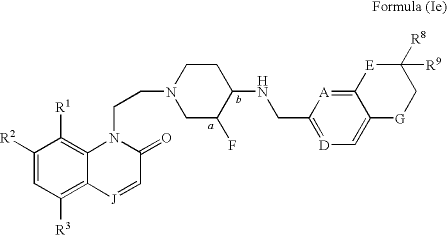

or a pharmaceutically acceptable salt thereof, wherein R¹, R², R³, R⁶, R⁸, R⁹, A, D, E, G, and J are as defined hereinabove, and wherein the compound of Formula (Ie) is substantially free of a cis (±) mixture of its enantiomers.
5.1) In one aspect of the compound of Formula (Ie), or a pharmaceutically acceptable salt thereof,

A, D, E, G, R⁸, and R⁹, together with the ring atoms to which they are attached, form a group selected from:

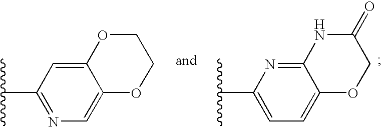

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
5.2) In another aspect of the compound of Formula (Ie), or a pharmaceutically acceptable salt thereof,

A, D, E, G, R⁸, and R⁹, together with the ring atoms to which they are attached, form a group selected from:

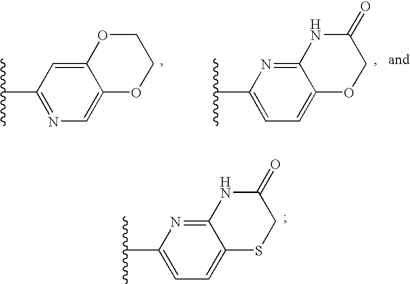

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
5.3) In still another aspect of the compound of Formula (Ie), or a pharmaceutically acceptable salt thereof,

A, D, E, G, R⁸, and R⁹, together with the ring atoms to which they are attached, form a group selected from:

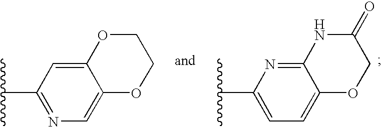

J is selected from N and CR⁴;

R¹ is H;

R² is selected from cyano and methoxy;

R³ is H; and

R⁴ is selected from H and methyl.
5.4) In yet another aspect of the compound of Formula (Ie), or a pharmaceutically acceptable salt thereof,

A, D, E, G, R⁸, and R⁹, together with the ring atoms to which they are attached, form a group selected from:

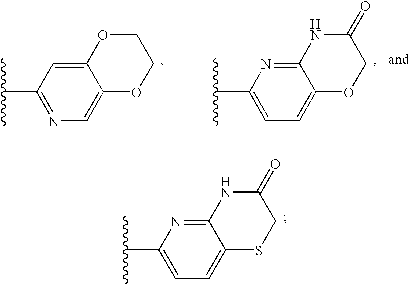

J is selected from N and CR⁴;

R¹ is H;

R² is selected from cyano and methoxy;

R³ is H; and

R⁴ is selected from H and methyl.
6) In yet a further aspect, the compound of Formula (I), or a pharmaceutically acceptable salt thereof, may be a compound of Formula (If):

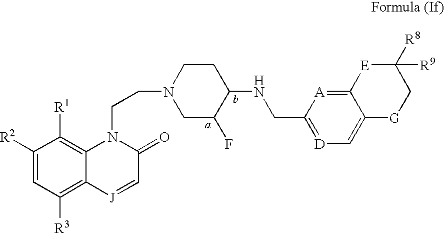

or a pharmaceutically acceptable salt thereof, wherein R¹, R², R³, R⁸, R⁹, A, D, E, G, and J are as defined hereinabove, wherein the fluoro group on carbon “a” and the —NH— group on carbon “b” are in a cis relationship to each other, and wherein the compound of Formula (If) is substantially free of a cis (±) mixture of its enantiomers.
6.1) In one aspect of the compound of Formula (If), or a pharmaceutically acceptable salt thereof,

A, D, E, G, R⁸, and R⁹, together with the ring atoms to which they are attached, form a group selected from:

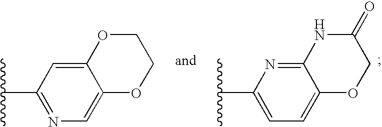

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
6.2) In another aspect of the compound of Formula (If), or a pharmaceutically acceptable salt thereof,

A, D, E, G, R⁸, and R⁹, together with the ring atoms to which they are attached, form a group selected from:

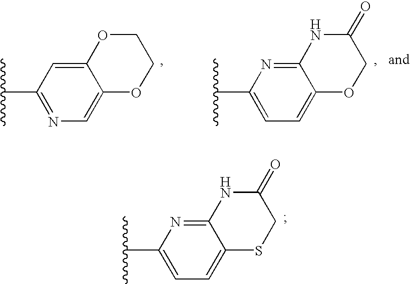

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
6.3) In still another aspect of the compound of Formula (If), or a pharmaceutically acceptable salt thereof,

A, D, E, G, R⁸, and R⁹, together with the ring atoms to which they are attached, form a group selected from:

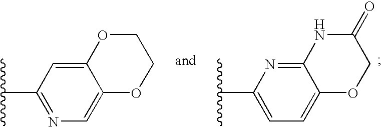

J is selected from N and CR⁴;

R¹ is H;

R² is selected from cyano and methoxy;

R³ is H; and

R⁴ is selected from H and methyl.
6.4) In yet another aspect of the compound of Formula (If), or a pharmaceutically acceptable salt thereof,

A, D, E, G, R⁸, and R⁹, together with the ring atoms to which they are attached, form a group selected from:

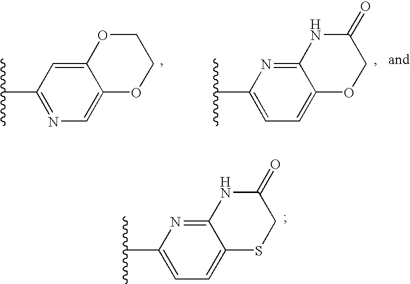

J is selected from N and CR⁴;

R¹ is H;

R² is selected from cyano and methoxy;

R³ is H; and

R⁴ is selected from H and methyl.
7) In one aspect, the compound of Formula (I), or a pharmaceutically acceptable salt thereof, may be a compound of Formula (Ig):

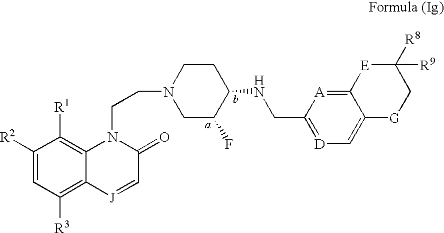

or a pharmaceutically acceptable salt thereof, wherein R¹, R², R³, R⁸, R⁹, A, D, E, G, and J are as defined hereinabove, wherein the compound of Formula (Ig) is substantially free of a cis (±) mixture of its enantiomers.
7.1) In one aspect of the compound of Formula (Ig), or a pharmaceutically acceptable salt thereof,

A, D, E, G, R⁸, and R⁹, together with the ring atoms to which they are attached, form a group selected from:

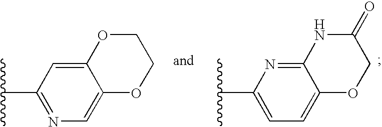

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
7.2) In another aspect of the compound of Formula (Ig), or a pharmaceutically acceptable salt thereof,

A, D, E, G, R⁸, and R⁹, together with the ring atoms to which they are attached, form a group selected from:

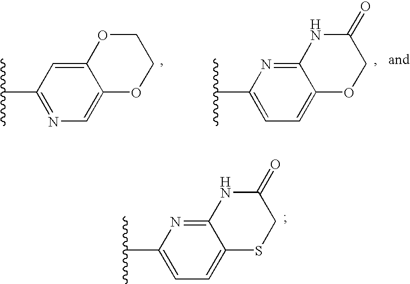

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
7.3) In still another aspect of the compound of Formula (Ig), or a pharmaceutically acceptable salt thereof,

A, D, E, G, R⁸, and R⁹, together with the ring atoms to which they are attached, form a group selected from:

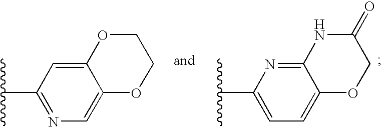

J is selected from N and CR⁴;

R¹ is H;

R² is selected from cyano and methoxy;

R³ is H; and

R⁴ is selected from H and methyl.
7.4) In yet another aspect of the compound of Formula (Ig), or a pharmaceutically acceptable salt thereof

A, D, E, G, R⁸, and R⁹, together with the ring atoms to which they are attached, form a group selected from:

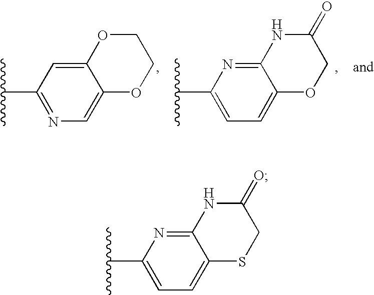

J is selected from N and CR⁴;

R¹ is H;

R² is selected from cyano and methoxy;

R³ is H; and

R⁴ is selected from H and methyl.
8) In another aspect, the compound of Formula (I) or a pharmaceutically acceptable salt thereof, may be a compound of Formula (Ih):

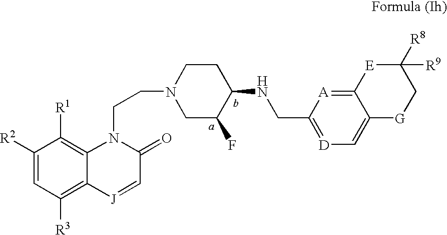

or a pharmaceutically acceptable salt thereof, wherein R¹, R², R³, R⁸, R⁹, A, D, E, G, and J are as defined hereinabove, wherein the compound of Formula (Ii) is substantially free of a cis (±) mixture of its enantiomers.
8.1) In one aspect of the compound of Formula (Ih), or a pharmaceutically acceptable salt thereof,

A, D, E, G, R⁸, and R⁹, together with the ring atoms to which they are attached, form a group selected from:

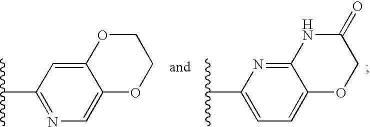

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
8.2) In another aspect of the compound of Formula (Ih), or a pharmaceutically acceptable salt thereof,

A, D, E, G, R⁸, and R⁹, together with the ring atoms to which they are attached, form a group selected from:

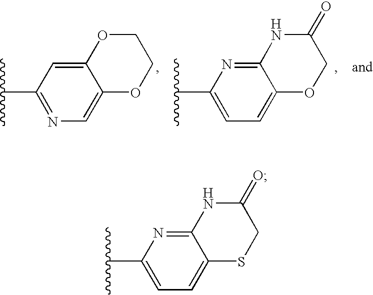

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
8.3) In still another aspect of the compound of Formula (Ih), or a pharmaceutically acceptable salt thereof,

A, D, E, G, R⁸, and R⁹, together with the ring atoms to which they are attached, form a group selected from:

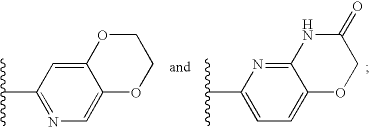

J is selected from N and CR⁴;

R¹ is H;

R² is selected from cyano and methoxy;

R³ is H; and

R⁴ is selected from H and methyl.
8.4) In yet another aspect of the compound of Formula (Ih), or a pharmaceutically acceptable salt thereof,

A, D, E, G, R⁸, and R⁹, together with the ring atoms to which they are attached, form a group selected from:

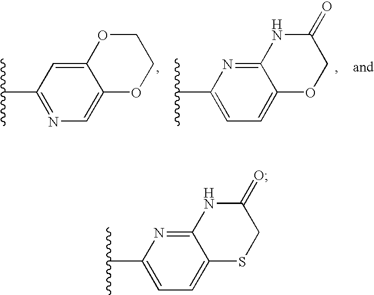

J is selected from N and CR⁴;

R¹ is H;

R² is selected from cyano and methoxy;

R³ is H; and

R⁴ is selected from H and methyl.

### Formula (II)

1.1 In one aspect of the compound of Formula (II), or a pharmaceutically acceptable salt thereof,

A, D, E, G, R⁸, and R⁹, together with the ring atoms to which they are attached, form a group selected from:

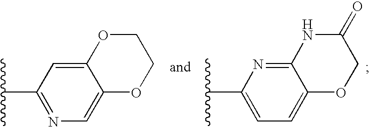

J is selected from N and CR⁴;

R¹ is H;

R² is selected from cyano and —OR^{2a};

R^{2a} is C₁₋₆alkyl;

R³ is H;

R⁴ is selected from H and C₁₋₆alkyl;

R⁶ is selected from fluoro and —OR^{6a}; and

R^{6a} is selected from H and C₁₋₆alkyl.
1.2) In another aspect of the compound of Formula (II), or a pharmaceutically acceptable salt thereof,

A, D, E, G, R⁸, and R⁹, together with the ring atoms to which they are attached, form a group selected from:

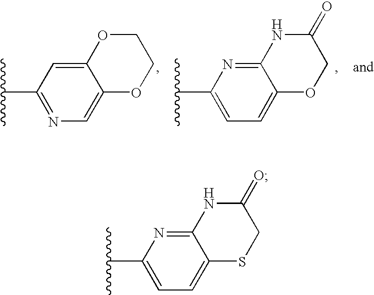

J is selected from N and CR⁴;

R¹ is H;

R² is selected from cyano and —OR^{2a};

R^{2a} is C₁₋₆alkyl;

R³ is H;

R⁴ is selected from H and C₁₋₆alkyl;

R⁶ is selected from fluoro and —OR^{6a}; and

R^{6a} is selected from H and C₁₋₆alkyl.
1.3) In still another aspect of the compound of Formula (II), or a pharmaceutically acceptable salt thereof,

A, D, E, G, R⁸, and R⁹, together with the ring atoms to which they are attached, form a group selected from:

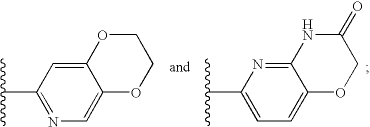

J is selected from N and CR⁴;

R¹ is H;

R² is selected from cyano and —OR^{2a};

R^{2a} is methyl;

R³ is H;

R⁴ is selected from H and methyl;

R⁶ is selected from fluoro and —OR^{6a}; and

R^{6a} is selected from H and methyl.
1.4) In yet another aspect of the compound of Formula (II), or a pharmaceutically acceptable salt thereof,

A, D, E, G, R⁸, and R⁹, together with the ring atoms to which they are attached, form a group selected from:

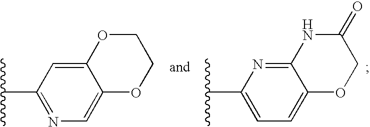

J is selected from N and CR⁴;

R¹ is H;

R² is selected from cyano and —OR^{2a};

R^{2a} is methyl;

R³ is H;

R⁴ is H;

R⁶ is selected from fluoro and —OR^{6a}; and

R^{6a} is selected from H and methyl.
1.5) In a further aspect of the compound of Formula (II), or a pharmaceutically acceptable salt thereof,

A, D, E, G, R⁸, and R⁹, together with the ring atoms to which they are attached, form a group selected from:

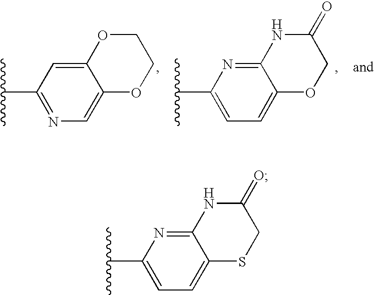

J is selected from N and CR⁴;

R¹ is H;

R² is selected from cyano and —OR^{2a};

R^{2a} is methyl;

R³ is H;

R⁴ is selected from H and methyl;

R⁶ is selected from fluoro and —OR^{6a}; and

R^{6a} is selected from H and methyl.
2) In one aspect, the compound of Formula (II), or a pharmaceutically acceptable salt thereof, may be a compound of Formula (IIa):

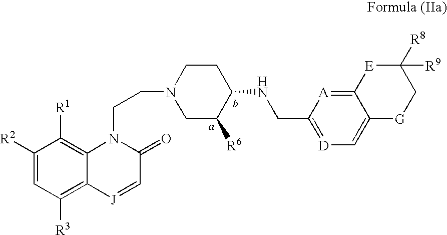

or a pharmaceutically acceptable salt thereof, wherein R¹, R², R³, R⁶, R⁸, R⁹, A, D, E, G, and J are as defined hereinabove.
2.1) In one aspect of the compound of Formula (IIa), or a pharmaceutically acceptable salt thereof,

A, D, E, G, R⁸, and R⁹, together with the ring atoms to which they are attached, form a group selected from:

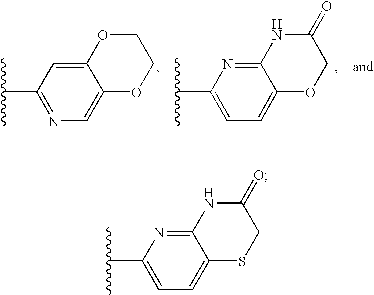

J is selected from N and CR⁴;

R¹ is H;

R² is selected from cyano and —OR^{2a};

R^{2a} is C₁₋₆alkyl;

R³ is H;

R⁴ is selected from H and C₁₋₆alkyl;

R⁶ is selected from fluoro and —OR^{6a}; and

R^{6a} is selected from H and C₁₋₆alkyl.
2.2) In another aspect of the compound of Formula (IIa), or a pharmaceutically acceptable salt thereof,

A, D, E, G, R⁸, and R⁹, together with the ring atoms to which they are attached, form a group selected from:

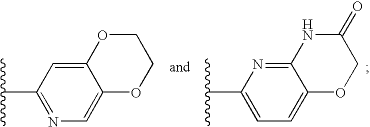

J is selected from N and CR⁴;

R¹ is H;

R² is selected from cyano and —OR^{2a};

R^{2a} is C₁₋₆alkyl;

R³ is H;

R⁴ is selected from H and C₁₋₆alkyl;

R⁶ is selected from fluoro and —OR^{6a}; and

R^{6a} is selected from H and C₁₋₆alkyl.
2.3) In still another aspect of the compound of Formula (Ha), or a pharmaceutically acceptable salt thereof,

A, D, E, G, R⁸, and R⁹, together with the ring atoms to which they are attached, form a group selected from:

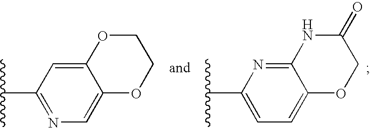

J is selected from N and CR⁴;

R¹ is H;

R² is selected from cyano and —OR^{2a};

R^{2a} is C₁₋₆alkyl;

R³ is H;

R⁴ is H;

R⁶ is selected from fluoro and —OR^{6a}; and

R^{6a} is selected from H and C₁₋₆alkyl.
2.4) In yet another aspect of the compound of Formula (Ha), or a pharmaceutically acceptable salt thereof,

A, D, E, G, R⁸, and R⁹, together with the ring atoms to which they are attached, form a group selected from:

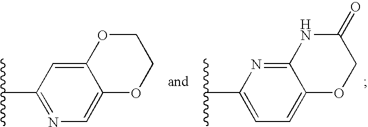

J is selected from N and CR⁴;

R¹ is H;

R² is selected from cyano and —OR^{2a};

R^{2a} is methyl;

R³ is H;

R⁴ is selected from H and methyl;

R⁶ is selected from fluoro and —OR^{6a}; and

R^{6a} is selected from H and methyl.
2.5) In a further aspect of the compound of Formula (Ha), or a pharmaceutically acceptable salt thereof,

A, D, E, G, R⁸, and R⁹, together with the ring atoms to which they are attached, form a group selected from:

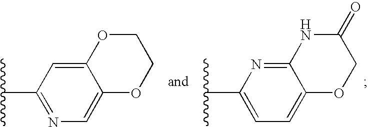

J is selected from N and CR⁴;

R¹ is H;

R² is selected from cyano and —OR^{2a};

R^{2a} is methyl;

R³ is H;

R⁴ is H;

R⁶ is selected from fluoro and —OR^{6a}; and

R^{6a} is selected from H and methyl.
3) In another aspect, the compound of Formula (II), or a pharmaceutically acceptable salt thereof, may be a compound of Formula (IIb):

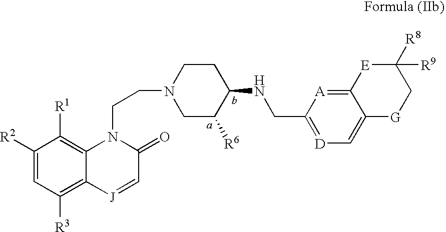

or a pharmaceutically acceptable salt thereof, wherein R¹, R², R³, R⁶, R⁸, R⁹, A, D, E, G, and J are as defined hereinabove.
3.1) In one aspect of the compound of Formula (IIb), or a pharmaceutically acceptable salt thereof,

A, D, E, G, R⁸, and R⁹, together with the ring atoms to which they are attached, form a group selected from:

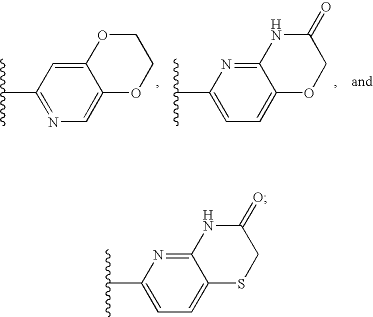

J is selected from N and CR⁴;

R¹ is H;

R² is selected from cyano and —OR^{2a};

R^{2a} is C₁₋₆alkyl;

R³ is H;

R⁴ is selected from H and C₁₋₆alkyl;

R⁶ is selected from fluoro and —OR^{6a}; and

R^{6a} is selected from H and C₁₋₆alkyl.
3.2) In another aspect of the compound of Formula (IIb), or a pharmaceutically acceptable salt thereof,

A, D, E, G, R⁸, and R⁹, together with the ring atoms to which they are attached, form a group selected from:

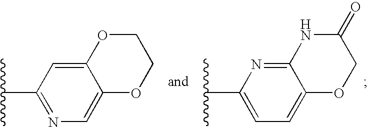

J is selected from N and CR⁴;

R¹ is H;

R² is selected from cyano and —OR^{2a};

R^{2a} is C₁₋₆alkyl;

R³ is H;

R⁴ is selected from H and C₁₋₆alkyl;

R⁶ is selected from fluoro and —OR^{6a}; and

R^{6a} is selected from H and C₁₋₆alkyl.
3.3) In still another aspect of the compound of Formula (IIb), or a pharmaceutically acceptable salt thereof,

A, D, E, G, R⁸, and R⁹, together with the ring atoms to which they are attached, form a group selected from:

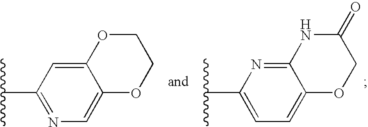

J is selected from N and CR⁴;

R¹ is H;

R² is selected from cyano and —OR^{2a};

R^{2a} is C₁₋₆alkyl;

R³ is H;

R⁴ is selected from H;

R⁶ is selected from fluoro and —OR^{6a}; and

R^{6a} is selected from H and C₁₋₆alkyl.
3.4) In yet another aspect of the compound of Formula (IIb), or a pharmaceutically acceptable salt thereof,

A, D, E, G, R⁸, and R⁹, together with the ring atoms to which they are attached, form a group selected from:

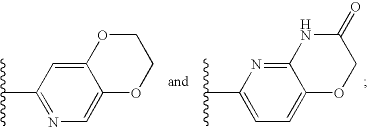

J is selected from N and CR⁴;

R¹ is H;

R² is selected from cyano and —OR^{2a};

R^{2a} is methyl;

R³ is H;

R⁴ is selected from H and methyl;

R⁶ is selected from fluoro and —OR^{6a}; and

R^{6a} is selected from H and methyl.
3.5) In a further aspect of the compound of Formula (lib), or a pharmaceutically acceptable salt thereof,

A, D, E, G, R⁸, and R⁹, together with the ring atoms to which they are attached, form a group selected from:

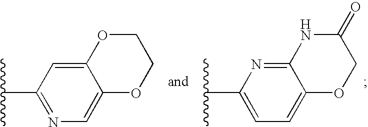

J is selected from N and CR⁴;

R¹ is H;

R² is selected from cyano and —OR^{2a};

R^{2a} is methyl;

R³ is H;

R⁴ is H;

R⁶ is selected from fluoro and —OR^{6a}; and

R^{6a} is selected from H and methyl.
4) In still another aspect, the compound of Formula (II), or a pharmaceutically acceptable salt thereof, may be a compound of Formula (IIc)

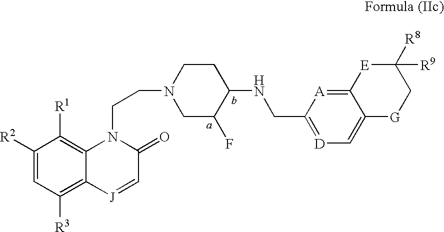

or a pharmaceutically acceptable salt thereof, wherein R¹, R², R³, R⁸, R⁹, A, D, E, G, and J are as defined hereinabove, and wherein the fluoro group on carbon “a” and the —NH— group on carbon “b” are in a trans relationship to each other.
4.1) In one aspect of the compound of Formula (IIc), or a pharmaceutically acceptable salt thereof,

A, D, E, G, R⁸, and R⁹, together with the ring atoms to which they are attached, form a group selected from:

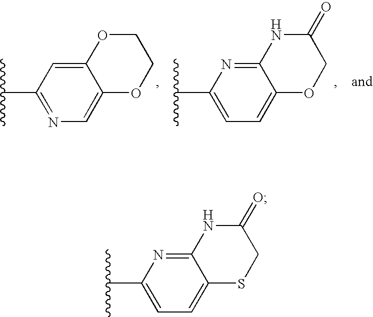

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
4.2) In another aspect of the compound of Formula (IIc), or a pharmaceutically acceptable salt thereof,

A, D, E, G, R⁸, and R⁹, together with the ring atoms to which they are attached, form a group selected from:

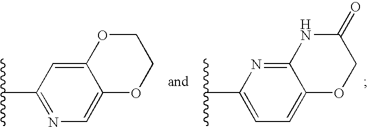

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
4.3) In still another aspect of the compound of Formula (IIc), or a pharmaceutically acceptable salt thereof,

A, D, E, G, R⁸, and R⁹, together with the ring atoms to which they are attached, form a group selected from:

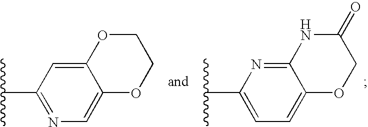

J is selected from N and CR⁴;

R¹ is H;

R² is selected from cyano and —OR^{2a};

R^{2a} is C₁₋₆alkyl;

R³ is H; and

R⁴ is H.
4.4) In yet another aspect of the compound of Formula (IIc), or a pharmaceutically acceptable salt thereof,

A, D, E, G, R⁸, and R⁹, together with the ring atoms to which they are attached, form a group selected from:

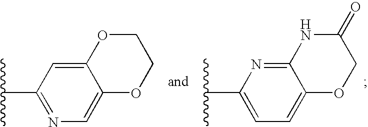

J is selected from N and CR⁴;

R¹ is H;

R² is selected from cyano and methoxy;

R³ is H; and

R⁴ is selected from H and methyl.
4.5) In a further aspect of the compound of Formula (IIc), or a pharmaceutically acceptable salt thereof,

A, D, E, G, R⁸, and R⁹, together with the ring atoms to which they are attached, form a group selected from:

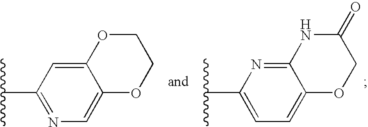

J is selected from N and CR⁴;

R¹ is H;

R² is selected from cyano and methoxy;

R³ is H; and

R⁴ is H.
5) In yet another aspect, the compound of Formula (II), or a pharmaceutically acceptable salt thereof, may be a compound of Formula (IId):

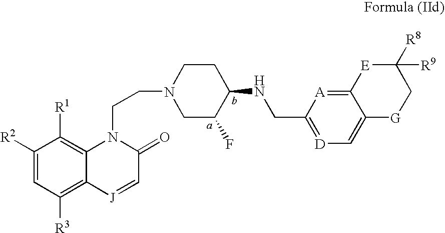

or a pharmaceutically acceptable salt thereof, wherein R¹, R², R³, R⁸, R⁹, A, D, E, G, and J are as defined hereinabove.
5.1) In one aspect of the compound of Formula (IId), or a pharmaceutically acceptable salt thereof,

A, D, E, G, R⁸, and R⁹, together with the ring atoms to which they are attached, form a group selected from:

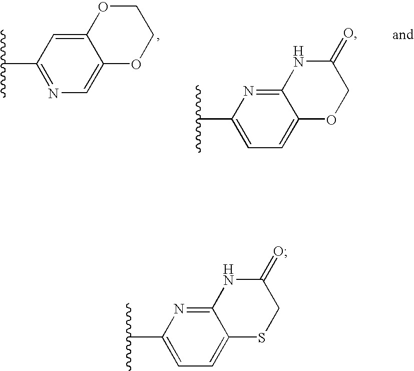

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
5.2) In another aspect of the compound of Formula (IId), or a pharmaceutically acceptable salt thereof,

A, D, E, G, R⁸, and R⁹, together with the ring atoms to which they are attached, form a group selected from:

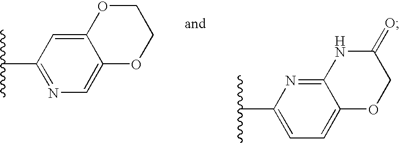

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
5.3) In still another aspect of the compound of Formula (IId), or a pharmaceutically acceptable salt thereof,

A, D, E, G, R⁸, and R⁹, together with the ring atoms to which they are attached, form a group selected from:

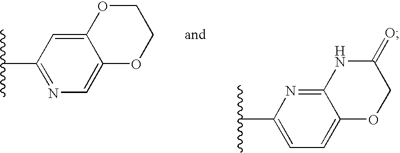

J is selected from N and CR⁴;

R¹ is H;

R² is selected from cyano and —OR^{2a};

R^{2a} is C₁₋₆alkyl;

R³ is H; and

R⁴ is H.
5.4) In yet another aspect of the compound of Formula (IId), or a pharmaceutically acceptable salt thereof,

A, D, E, G, R⁸, and R⁹, together with the ring atoms to which they are attached, form a group selected from:

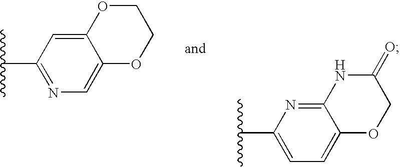

J is selected from N and CR⁴;

R¹ is H;

R² is selected from cyano and methoxy;

R³ is H; and

R⁴ is selected from H and methyl.
5.5) In a further aspect of the compound of Formula (IId), or a pharmaceutically acceptable salt thereof,

A, D, E, G, R⁸, and R⁹, together with the ring atoms to which they are attached, form a group selected from:

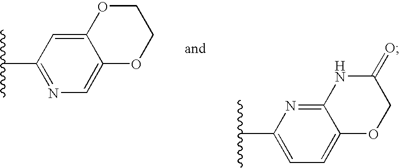

J is selected from N and CR⁴;

R¹ is H;

R² is selected from cyano and methoxy;

R³ is H; and

R⁴ is H.
6) In a further aspect, the compound of Formula (II), or a pharmaceutically acceptable salt thereof, may be a compound of Formula (IIe):

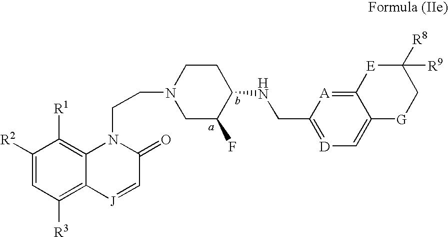

or a pharmaceutically acceptable salt thereof, wherein R¹, R², R³, R⁸, R⁹, A, D, E, G, and J are as defined hereinabove.
6.1) In one aspect of the compound of Formula (He), or a pharmaceutically acceptable salt thereof,

A, D, E, G, R⁸, and R⁹, together with the ring atoms to which they are attached, form a group selected from:

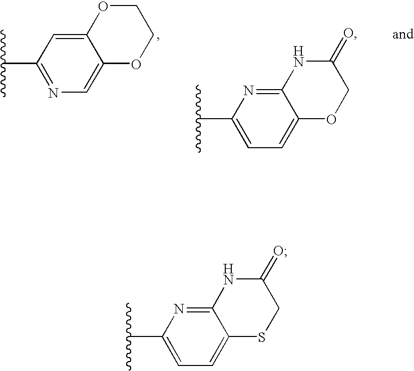

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
6.2) In another aspect of the compound of Formula (IIe), or a pharmaceutically acceptable salt thereof,

A, D, E, G, R⁸, and R⁹, together with the ring atoms to which they are attached, form a group selected from:

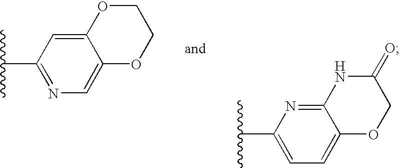

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
6.3) In still another aspect of the compound of Formula (lie), or a pharmaceutically acceptable salt thereof,

A, D, E, G, R⁸, and R⁹, together with the ring atoms to which they are attached, form a group selected from:

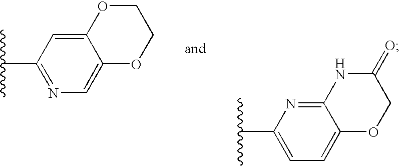

J is selected from N and CR⁴;

R¹ is H;

R² is selected from cyano and —OR^{2a};

R^{2a} is C₁₋₆alkyl;

R³ is H; and

R⁴ is H.
6.4) In yet another aspect of the compound of Formula (lie), or a pharmaceutically acceptable salt thereof,

A, D, E, G, R⁸, and R⁹, together with the ring atoms to which they are attached, form a group selected from:

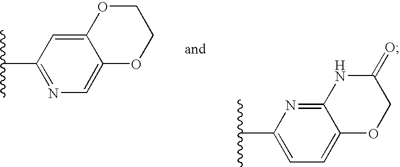

J is selected from N and CR⁴;

R¹ is H;

R² is selected from cyano and methoxy;

R³ is H; and

R⁴ is selected from H and methyl.
6.5) In a further aspect of the compound of Formula (IIe), or a pharmaceutically acceptable salt thereof,

A, D, E, G, R⁸, and R⁹, together with the ring atoms to which they are attached, form a group selected from:

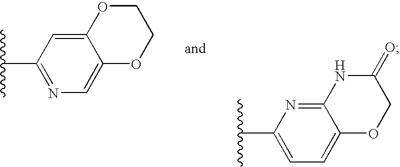

J is selected from N and CR⁴;

R¹ is H;

R² is selected from cyano and methoxy;

R³ is H; and

R⁴ is methyl.
7) In still a further aspect, the compound of Formula (II), or a pharmaceutically acceptable salt thereof, may be a compound of Formula (IIf):

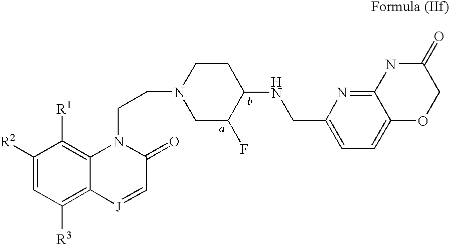

or a pharmaceutically acceptable salt thereof, wherein R¹, R², R³, and J are as defined hereinabove, and wherein the fluoro group on carbon “a” and the —NH— group on carbon “b” are in a trans relationship to each other.
7.1) In one aspect of the compound of Formula (IIf), or a pharmaceutically acceptable salt thereof,

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
7.2) In another aspect of the compound of Formula (IIf), or a pharmaceutically acceptable salt thereof,

J is selected from N and CR⁴;

R¹ is H;

R² is selected from cyano and —OR^{2a};

R^{2a} is C₁₋₆alkyl;

R³ is H; and

R⁴ is H.
7.3) In still another aspect of the compound of Formula (IIf), or a pharmaceutically acceptable salt thereof,

J is selected from N and CR⁴;

R¹ is H;

R² is selected from cyano and methoxy;

R³ is H; and

R⁴ is selected from H and methyl.
7.4) In yet another aspect of the compound of Formula (IIf), or a pharmaceutically acceptable salt thereof,

J is selected from N and CR⁴;

R¹ is H;

R² is selected from cyano and methoxy;

R³ is H; and

R⁴ is H.
8) In yet a further aspect, the compound of Formula (IIg), or a pharmaceutically acceptable salt thereof, may be a compound of Formula (IIg):

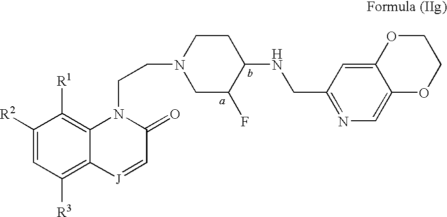

or a pharmaceutically acceptable salt thereof, wherein R¹, R², R³, and J are as defined hereinabove, and wherein the fluoro group on carbon “a” and the —NH— group on carbon “b” are in a trans relationship to each other.
8.1) In one aspect of the compound of Formula (IIg), or a pharmaceutically acceptable salt thereof,

J is selected from N and CR⁴;

R¹ is H;

R² is selected from cyano and —OR^{2a};

R^{2a} is C₁₋₆alkyl;

R³ is H; and

R⁴ is selected from H and C₁₋₆alkyl.
8.2) In another aspect of the compound of Formula (IIg), or a pharmaceutically acceptable salt thereof,

J is selected from N and CR⁴;

R¹ is H;

R² is selected from cyano and —OR^{2a};

R^{2a} is C₁₋₆alkyl;

R³ is H; and

R⁴ is H.
8.3) In still another aspect of the compound of Formula (IIg), or a pharmaceutically acceptable salt thereof,

J is selected from N and CR⁴;

R¹ is H;

R² is selected from cyano and methoxy;

R³ is H; and

R⁴ is selected from H and methyl.
8.4) In yet another aspect of the compound of Formula (IIg), or a pharmaceutically acceptable salt thereof,

J is selected from N and CR⁴;

R¹ is H;

R² is selected from cyano and methoxy;

R³ is H; and

R⁴ is H.

In another aspect, the present invention provides compounds of Formulas (I) and (II) and pharmaceutically acceptable salts thereof, as illustrated by the Examples, each of which provides a further independent aspect of the invention.

### Biological Activity

The compounds of Formulas (I) (II) and pharmaceutically acceptable salts thereof are of interest due to their antibacterial effects. The ability of the invention compounds disclosed herein to achieve an antibacterial effect may be demonstrated by the following tests.

### Bacterial Susceptibility Testing Methods 1

Compounds may be tested for antimicrobial activity by susceptibility testing in liquid media in a 96 well format. Compounds may be dissolved in dimethylsulfoxide and tested in 10 doubling dilutions in the susceptibility assays. The organisms used in the assay may be grown overnight on suitable agar media and then suspended in a liquid medium appropriate for the growth of the organism. The suspension may be adjusted to be equal to a 0.5 McFarland and a further 1 in 10 dilution may be made into the same liquid medium to prepare the final organism suspension in 100 μL. Plates may be incubated under appropriate conditions at 37° C. for 24 hours prior to reading. The Minimum Inhibitory Concentration (MIC) was determined as the lowest drug concentration able to reduce growth by 80% or more.

Compounds were evaluated against a panel of Gram-positive species, including Staphylococcus aureus, Streptococcus pneumoniae, Streptococcus pyogenes, and Enterococcus faecium. In addition, compounds were evaluated against a panel of Gram-negative species including Haemophilus influenzae, Escherichia coli and Moraxella catarrhalis. Compounds of the present invention and pharmaceutically acceptable salts thereof are believed to generally have MIC's less than or equal to 8 μg/ml versus one or more of the organisms named above.

The compound of Example 2 had an MIC of 0.25 (mg/L) against Staphylococcus aureus and an MIC of 0.5 (mg/L) against Escherichia coli.

### Bacterial Susceptibility Testing Methods 2

Compounds may be tested for antimicrobial activity by susceptibility testing using microbroth dilution methods recommended by CSLI. Compounds may be dissolved in dimethylsulfoxide and tested in 10 doubling dilutions in the susceptibility assays such that the final dimethylsulfoxide concentration in the assay is 2% (v/v). The Staphylococcus aureus (Sau) 516 cells used in the assay may be grown overnight on appropriate agar media and then suspended in the NCCLS-recommended liquid susceptibility-testing media. The turbidity of each suspension may be adjusted to be equal to a 0.5 McFarland standard, and a further 1-in-200 dilution may be made into the same liquid medium to prepare the final organism suspension, and 100 μL of this suspension may be added to each well of a microtiter plate containing compound dissolved in 2 μL, of dimethylsulfoxide. Plates may be incubated under appropriate conditions of atmosphere and temperature and for times according to NCCLS standard methods prior to being read. As used in Table 1 below, the term “inhibition” refers to the percentage of Staphylococcus aureus 516 cell growth that is suppressed (compared to an untreated sample) by the compound of the indicated example number.

When tested in the above in-vitro assay, the compounds and salts of the example numbers listed below were tested at the indicated concentrations, providing the indicated inhibitions. In most instances, the highest concentrations at which the compounds were tested was 8 μg/mL. In some instances, however, compounds were tested at concentrations higher than 8 μg/mL, typically when the concentration was first measured in μM and was subsequently converted to μg/mL. In those cases in which compounds were tested at more than one concentration higher than 8 μg/mL, the concentration closest to 8 μg/mL is provided below.

TABLE 1

| | Tested | |
|---|---|---|
| Example | Concentration | Inhibition |
| Number | (μg/mL) | (%) |
| 1 | 8 | 100 |
| 2 | 8 | 100 |
| 3 | 8 | 99 |
| 4 | 8 | 100 |
| 5 | 8 | 100 |
| 6 | 8 | 99 |
| 7 | 8 | 100 |
| 8 | 8 | 99 |
| 9 | 8 | 100 |
| 10 | 8 | 100 |

Examples 11 to 20

| | | |
|---|---|---|
| 11 | 8 | 100 |
| 12 | 8 | 100 |
| 13 | 8 | 100 |
| 14 | 8 | 100 |
| 15 | 8 | 97 |
| 16 | 8 | 97 |
| 17 | 8 | 99 |
| 18 | 8 | 95 |
| 19 | 8 | 100 |
| 20 | 8 | 100 |

Examples 21 to 30

| | | |
|---|---|---|
| 21 | 8 | 100 |
| 22 | 8 | 100 |
| 23 | 8 | 100 |
| 24 | 8 | 100 |
| 25 | 8 | 98 |
| 26 | 8 | 98 |
| 27 | 8 | 87 |
| 28 | 8 | 98 |
| 29 | 8 | 84 |
| 30 | 8 | 98 |

Examples 31 to 40

| | | |
|---|---|---|
| 31 | 8 | 87 |
| 32 | 8 | 98 |
| 33 | 13 | 96 |
| 34 | 12 | 91 |
| 35 | 14 | 100 |
| 36 | 12 | 90 |
| 37 | 13 | 100 |
| 38 | 12 | 100 |
| 39 | 14 | 100 |
| 40 | 12 | 100 |

Examples 41-52

| | | |
|---|---|---|
| 41 | 8 | 100 |
| 42 | 8 | 100 |
| 43 | 4 | 100* |
| 44 | 8 | 100 |
| 45 | 8 | 97 |
| 46 | 8 | 100 |
| 47 | 8 | 100 |
| 50 | 14 | 100 |
| 51 | 9 | 100 |
| 52 | 13 | 100 |

*The inhibition for the compound of Example 43 at a concentration of 8 μg/mL was 12%, though it is believed that the 12% figure is erroneous.

Thus, in one aspect there is provided a compound of Formula (I) or (II), or a pharmaceutically acceptable salt thereof, for use as a medicament.

In still another aspect, there is provided the use a compound of Formula (I) or (II), or a pharmaceutically acceptable salt thereof, in the manufacture of a medicament for use in the treatment of a bacterial infection in a warm-blooded animal such as man.

In yet another aspect, there is provided the use of a compound of Formula (I) or (II), or a pharmaceutically acceptable salt thereof, in the manufacture of a medicament for use in the treatment of a bacterial infection caused by one or more pathogenic organisms such as Acinetobacter baumanii, Aeromis hydrophila, Bacillus anthracis, Bacteroides fragilis, Bordatella pertussis, Burkholderia cepacia, Chlamyida pneumoniae, Citrobacter freundii, Clostridium difficile, Enterobacter cloacae, Enterococcus faecalis, Enterococcus faecium, Enterobacter aerogenes, Escherichia coli, Fusobacterium necrophorum, Haemophilus influenzae, Haemophilus parainfluenzae, Haemophilus somnus, Klebsiella oxytoca, Klebsiella pneumoniae, Legionella pneumophila, Listeria monocytogenes, Moraxella catarrhalis, Morganella morganii, Mycoplasma pneumoniae, Neisseria gonorrhoeae, Neisseria meningitidis, Pasteurella multocida, Proteus mirabilis, Proteus vulgaris, Pseudomonas aeruginosa, Salmonella typhi, Salmonella typhimurium, Serratia marcesens, Shigella flexneria, Shigella dysenteriae, Staphylococcus aureus, Staphylococcus epidermidis, Staphylococcus haemolyticus, Staphylococcus intermedius, Staphylococcus saprophyticus, Stenotrophomonas maltophila, Streptococcus agalactiae, Streptococcus mutans, Streptococcus pneumoniae, and Streptococcus pyrogenes, in a warm-blooded animal such as man.

In a further aspect, there is provided the use of a compound of Formula (I) or (II), or a pharmaceutically acceptable salt thereof, in the manufacture of a medicament for use in the treatment of an infection such as bronchitis, C. difficile colitis, cervicitis, endocarditis, gonococcal urethritis, inhalation Anthrax, intra-abdominal infections, meningitis, osteomyelitis, otitis media, pharyngitis, pneumonia, prostatitis, septicemia, sinusitis, skin and soft tissue infections, and urinary tract infections, in a warm-blooded animal such as man.

In still a further aspect, there is provided the use of a compound of Formula (I) or (II), or a pharmaceutically acceptable salt thereof, in the manufacture of a medicament for use in the treatment of a bacterial infection, wherein the bacteria is of a genus selected from Aeromonas, Acinetobacter, Bacillus, Bacteroides, Bordetella, Burkholderia, Chlamydophila, Citrobacter, Clostridium, Enterobacter, Enterococcus, Escherichia, Flavobacterium, Fusobacterium, Haemophilus, Klebsiella, Legionella, Listeria, Morganella, Moraxella, Mycoplasma, Neisseria, Pasteurella, Peptococci, Peptostreptococci, Prevotella, Proteus Salmonella, Pseudomonas, Serratia, Shigella, Stenotrophomonas, Streptococcus, and Staphylococcus, in a warm-blooded animal such as man.

In another aspect, there is provided a method for treating a bacterial infection in a warm-blooded animal such as man, said method comprising administering to said animal an effective amount of a compound of Formula (I) or (II), or a pharmaceutically acceptable salt thereof.

In still another aspect, there is provided a method for treating a bacterial infection caused by one or more pathogenic organisms such as Acinetobacter baumanii, Aeromis hydrophila, Bacillus anthracis, Bacteroides fragilis, Bordatella pertussis, Burkholderia cepacia, Chlamyida pneumoniae, Citrobacter freundii, Clostridium difficile, Enterobacter cloacae, Enterococcus faecalis, Enterococcus faecium, Enterobacter aerogenes, Escherichia coli, Fusobacterium necrophorum, Haemophilus influenzae, Haemophilus parainfluenzae, Haemophilus somnus, Klebsiella oxytoca, Klebsiella pneumoniae, Legionella pneumophila, Listeria monocytogenes, Moraxella catarrhalis, Morganella morganii, Mycoplasma pneumoniae, Neisseria gonorrhoeae, Neisseria meningitidis, Pasteurella multocida, Proteus mirabilis, Proteus vulgaris, Pseudomonas aeruginosa, Salmonella typhi, Salmonella typhimurium, Serratia marcesens, Shigella flexneria, Shigella dysenteriae, Staphylococcus aureus, Staphylococcus epidermidis, Staphylococcus haemolyticus, Staphylococcus intermedius, Staphylococcus saprophyticus, Stenotrophomonas maltophila, Streptococcus agalactiae, Streptococcus mutans, Streptococcus pneumoniae, and Streptococcus pyrogenes, in a warm-blooded animal such as man, said method comprising administering to said animal an effective amount of a compound of Formula (I) or (II), or a pharmaceutically acceptable salt thereof.

In yet another aspect, there is provided a method for treating a bacterial infection such as bronchitis, C. difficile colitis, cervicitis, endocarditis, gonococcal urethritis, inhalation Anthrax, intra-abdominal infections, meningitis, osteomyelitis, otitis media, pharyngitis, pneumonia, prostatitis, septicemia, sinusitis, skin and soft tissue infections, and urinary tract infections, in a warm-blooded animal such as man, said method comprising administering to said animal an effective amount of a compound of Formula (I) or (II), or a pharmaceutically acceptable salt thereof.

In a further aspect, there is provided a method for treating a bacterial infection, wherein the bacteria is of a genus selected from Aeromonas, Acinetobacter, Bacillus, Bacteroides, Bordetella, Burkholderia, Chlamydophila, Citrobacter, Clostridium, Enterobacter, Enterococcus, Escherichia, Flavobacterium, Fusobacterium, Haemophilus, Klebsiella, Legionella, Listeria, Morganella, Moraxella, Mycoplasma, Neisseria, Pasteurella, Peptococci, Peptostreptococci, Prevotella, Proteus Salmonella, Pseudomonas, Serratia, Shigella, Stenotrophomonas, Streptococcus, and Staphylococcus, in a warm-blooded animal such as man, said method comprising administering to said animal an effective amount of a compound of Formula (I) or (II), or a pharmaceutically acceptable salt thereof.

In still a further aspect, there is provided a compound of Formula (I) or (II), or a pharmaceutically acceptable salt thereof, for use in treating a bacterial infection in a warm-blooded animal, such as man.

In another aspect, there is provided a compound of Formula (I) or (II), or a pharmaceutically acceptable salt thereof, for use in treating a bacterial infection caused by one or more pathogenic organisms such as Acinetobacter baumanii, Aeromis hydrophila, Bacillus anthracis, Bacteroides fragilis, Bordatella pertussis, Burkholderia cepacia, Chlamyida pneumoniae, Citrobacter freundii, Clostridium difficile, Enterobacter cloacae, Enterococcus faecalis, Enterococcus faecium, Enterobacter aerogenes, Escherichia coli, Fusobacterium necrophorum, Haemophilus influenzae, Haemophilus parainfluenzae, Haemophilus somnus, Klebsiella oxytoca, Klebsiella pneumoniae, Legionella pneumophila, Listeria monocytogenes, Moraxella catarrhalis, Morganella morganii, Mycoplasma pneumoniae, Neisseria gonorrhoeae, Neisseria meningitidis, Pasteurella multocida, Proteus mirabilis, Proteus vulgaris, Pseudomonas aeruginosa, Salmonella typhi, Salmonella typhimurium, Serratia marcesens, Shigella flexneria, Shigella dysenteriae, Staphylococcus aureus, Staphylococcus epidermidis, Staphylococcus haemolyticus, Staphylococcus intermedius, Staphylococcus saprophyticus, Stenotrophomonas maltophila, Streptococcus agalactiae, Streptococcus mutans, Streptococcus pneumoniae, and Streptococcus pyrogenes, in a warm-blooded animal such as man.

In still another aspect, there is provided a compound of Formula (I) or (II), or a pharmaceutically acceptable salt thereof, for use in treating infections such as bronchitis, C. difficile colitis, cervicitis, endocarditis, gonococcal urethritis, inhalation Anthrax, intra-abdominal infections, meningitis, osteomyelitis, otitis media, pharyngitis, pneumonia, prostatitis, septicemia, sinusitis, skin and soft tissue infections, and urinary tract infections, in a warm-blooded animal such as man.

In yet another aspect, there is provided a compound of Formula (I) or (II), or a pharmaceutically acceptable salt thereof, for use in treating a bacterial infection, wherein the bacteria is of a genus selected from Aeromonas, Acinetobacter, Bacillus, Bacteroides, Bordetella, Burkholderia, Chlamydophila, Citrobacter, Clostridium, Enterobacter, Enterococcus, Escherichia, Flavobacterium, Fusobacterium, Haemophilus, Klebsiella, Legionella, Listeria, Morganella, Moraxella, Mycoplasma, Neisseria, Pasteurella, Peptococci, Peptostreptococci, Prevotella, Proteus Salmonella, Pseudomonas, Serratia, Shigella, Stenotrophomonas, Streptococcus, and Staphylococcus, and Munnheimia, in a warm-blooded animal such as man.

In one aspect, the terms “infection” and “bacterial infection” may refer to a bacterial infection caused by Acinetobacter baumanii. In another aspect, the terms “infection” and “bacterial infection” may refer to a bacterial infection caused by Aeromis hydrophila. In still another aspect, the terms “infection” and “bacterial infection” may refer to a bacterial infection caused by Bacillus anthracis. In yet another aspect, the terms “infection” and “bacterial infection” may refer to a bacterial infection caused by Bacteroides fragilis. In a further aspect, the terms “infection” and “bacterial infection” may refer to a bacterial infection caused by Bordatella pertussis. In still a further aspect, the terms “infection” and “bacterial infection” may refer to a bacterial infection caused by Burkholderia cepacia. In yet a further aspect, the terms “infection” and “bacterial infection” may refer to a bacterial infection caused by Chlamyida pneumoniae. In one aspect, the terms “infection” and “bacterial infection” may refer to a bacterial infection caused by Citrobacter freundii. In another aspect, the terms “infection” and “bacterial infection” may refer to a bacterial infection caused by Clostridium difficile. In still another aspect, the terms “infection” and “bacterial infection” may refer to a bacterial infection caused by Enterobacter cloacae. In yet another aspect, the terms “infection” and “bacterial infection” may refer to a bacterial infection caused by Enterococcus faecalis. In a further aspect, the terms “infection” and “bacterial infection” may refer to a bacterial infection caused by Enterococcus faecium. In still a further aspect, the terms “infection” and “bacterial infection” may refer to a bacterial infection caused by Enterobacter aerogenes. In yet a further aspect, the terms “infection” and “bacterial infection” may refer to a bacterial infection caused by Escherichia coli. In one aspect, the terms “infection” and “bacterial infection” may refer to a bacterial infection caused by Fusobacterium necrophorum. In another aspect, the terms “infection” and “bacterial infection” may refer to a bacterial infection caused by Haemophilus influenzae. In still another aspect, the terms “infection” and “bacterial infection” may refer to a bacterial infection caused by Haemophilus parainfluenzae. In yet another aspect, the terms “infection” and “bacterial infection” may refer to a bacterial infection caused by Haemophilus somnus. In a further aspect, the terms “infection” and “bacterial infection” may refer to a bacterial infection caused by Klebsiella oxytoca. In still a further aspect, the terms “infection” and “bacterial infection” may refer to a bacterial infection caused by Klebsiella pneumoniae. In yet a further aspect, the terms “infection” and “bacterial infection” may refer to a bacterial infection caused by Legionella pneumophila. In one aspect, the terms “infection” and “bacterial infection” may refer to a bacterial infection caused by Listeria monocytogenes. In another aspect, the terms “infection” and “bacterial infection” may refer to a bacterial infection caused by Moraxella catarrhalis. In still another aspect, the terms “infection” and “bacterial infection” may refer to a bacterial infection caused by Morganella morganii. In yet another aspect, the terms “infection” and “bacterial infection” may refer to a bacterial infection caused by Mycoplasma pneumoniae. In a further aspect, the terms “infection” and “bacterial infection” may refer to a bacterial infection caused by Neisseria gonorrhoeae. In still a further aspect, the terms “infection” and “bacterial infection” may refer to a bacterial infection caused by Neisseria meningitidis. In yet a further aspect, the terms “infection” and “bacterial infection” may refer to a bacterial infection caused by Pasteurella multocida. In one aspect, the terms “infection” and “bacterial infection” may refer to a bacterial infection caused by Proteus mirabilis. In another aspect, the terms “infection” and “bacterial infection” may refer to a bacterial infection caused by Proteus vulgaris. In still another aspect, the terms “infection” and “bacterial infection” may refer to a bacterial infection caused by Pseudomonas aeruginosa. In yet another aspect, the terms “infection” and “bacterial infection” may refer to a bacterial infection caused by Salmonella typhi. In a further aspect, the terms “infection” and “bacterial infection” may refer to a bacterial infection caused by Salmonella typhimurium. In still a further aspect, the terms “infection” and “bacterial infection” may refer to a bacterial infection caused by Serratia marcesens. In yet a further aspect, the terms “infection” and “bacterial infection” may refer to a bacterial infection caused by Shigella flexneria. In one aspect, the terms “infection” and “bacterial infection” may refer to a bacterial infection caused by Shigella dysenteriae. In another aspect, the terms “infection” and “bacterial infection” may refer to a bacterial infection caused by Staphylococcus aureus. In still another aspect, the terms “infection” and “bacterial infection” may refer to a bacterial infection caused by Staphylococcus epidermidis. In yet another aspect, the terms “infection” and “bacterial infection” may refer to a bacterial infection caused by Staphylococcus haemolyticus. In a further aspect, the terms “infection” and “bacterial infection” may refer to a bacterial infection caused by Staphylococcus intermedius. In still a further aspect, the terms “infection” and “bacterial infection” may refer to a bacterial infection caused by Staphylococcus saprophyticus. In yet a further aspect, the terms “infection” and “bacterial infection” may refer to a bacterial infection caused by Stenotrophomonas maltophila. In one aspect, the terms “infection” and “bacterial infection” may refer to a bacterial infection caused by Streptococcus agalactiae. In another aspect, the terms “infection” and “bacterial infection” may refer to a bacterial infection caused by Streptococcus mutans. In a still another aspect, the terms “infection” and “bacterial infection” may refer to a bacterial infection caused by Streptococcus pneumoniae. In yet another aspect, the terms “infection” and “bacterial infection” may refer to a bacterial infection caused by Streptococcus pyrogenes.

In one aspect, the terms “infection and “bacterial infection” may refer to a bacterial infection caused by a bacteria of the genus Aeromonas. In another aspect, the terms “infection and “bacterial infection” may refer to a bacterial infection caused by a bacteria of the genus Acinetobacter. In still another aspect, the terms “infection and “bacterial infection” may refer to a bacterial infection caused by a bacteria of the genus Bacillus. In yet another aspect, the terms “infection and “bacterial infection” may refer to a bacterial infection caused by a bacteria of the genus Bacteroides. In a further aspect, the terms “infection and “bacterial infection” may refer to a bacterial infection caused by a bacteria of the genus Bordetella. In still a further aspect, the terms “infection and “bacterial infection” may refer to a bacterial infection caused by a bacteria of the genus Burkholderia. In yet a further aspect, the terms “infection and “bacterial infection” may refer to a bacterial infection caused by a bacteria of the genus Chlamydophila. In one aspect, the terms “infection and “bacterial infection” may refer to a bacterial infection caused by a bacteria of the genus Citrobacter. In another aspect, the terms “infection and “bacterial infection” may refer to a bacterial infection caused by a bacteria of the genus Clostridium. In still another aspect, the terms “infection and “bacterial infection” may refer to a bacterial infection caused by a bacteria of the genus Enterobacter. In yet another aspect, the terms “infection and “bacterial infection” may refer to a bacterial infection caused by a bacteria of the genus Enterococcus. In a further aspect, the terms “infection and “bacterial infection” may refer to a bacterial infection caused by a bacteria of the genus Escherichia. In still a further aspect, the terms “infection and “bacterial infection” may refer to a bacterial infection caused by a bacteria of the genus Flavobacterium. In yet a further aspect, the terms “infection and “bacterial infection” may refer to a bacterial infection caused by a bacteria of the genus Fusobacterium. In one aspect, the terms “infection and “bacterial infection” may refer to a bacterial infection caused by a bacteria of the genus Haemophilus. In one aspect, the terms “infection and “bacterial infection” may refer to a bacterial infection caused by a bacteria of the genus Klebsiella. In another aspect, the terms “infection and “bacterial infection” may refer to a bacterial infection caused by a bacteria of the genus Legionella. In still another aspect, the terms “infection and “bacterial infection” may refer to a bacterial infection caused by a bacteria of the genus Listeria. In yet another aspect, the terms “infection and “bacterial infection” may refer to a bacterial infection caused by a bacteria of the genus Morganella. In a further aspect, the terms “infection and “bacterial infection” may refer to a bacterial infection caused by a bacteria of the genus Moraxella. In still a further aspect, the terms “infection and “bacterial infection” may refer to a bacterial infection caused by a bacteria of the genus Mycoplasma. In yet a further aspect, the terms “infection and “bacterial infection” may refer to a bacterial infection caused by a bacteria of the genus Neisseria. In one aspect, the terms “infection and “bacterial infection” may refer to a bacterial infection caused by a bacteria of the genus Pasteurella. In another aspect, the terms “infection and “bacterial infection” may refer to a bacterial infection caused by a bacteria of the genus Peptococci. In still another aspect, the terms “infection and “bacterial infection” may refer to a bacterial infection caused by a bacteria of the genus Peptostreptococci. In yet another aspect, the terms “infection and “bacterial infection” may refer to a bacterial infection caused by a bacteria of the genus Prevotella. In a further aspect, the terms “infection and “bacterial infection” may refer to a bacterial infection caused by a bacteria of the genus Proteus. In still a further aspect, the terms “infection and “bacterial infection” may refer to a bacterial infection caused by a bacteria of the genus Pseudomonas. In still another aspect, the terms “infection and “bacterial infection” may refer to a bacterial infection caused by a bacteria of the genus Salmonella. In yet a further aspect, the terms “infection and “bacterial infection” may refer to a bacterial infection caused by a bacteria of the genus Serratia. In one aspect, the terms “infection and “bacterial infection” may refer to a bacterial infection caused by a bacteria of the genus Shigella. In yet another aspect, the terms “infection and “bacterial infection” may refer to a bacterial infection caused by a bacteria of the genus Staphylococcus. In another aspect, the terms “infection and “bacterial infection” may refer to a bacterial infection caused by a bacteria of the genus Stenotrophomonas. In still another aspect, the terms “infection and “bacterial infection” may refer to a bacterial infection caused by a bacteria of the genus Streptococcus.

In one aspect, the terms “infection” and “bacterial infection” may refer to a gynecological infection. In another aspect the terms “infection” and “bacterial infection” may refer to a respiratory tract infection (RTI). In still another, the terms “infection” and “bacterial infection” may refer to a sexually transmitted disease. In yet another aspect, the terms “infection” and “bacterial infection” may refer to a urinary tract infection. In a further aspect, the terms “infection” and “bacterial infection” may refer to acute exacerbation of chronic bronchitis (ACEB). In yet a further aspect, the terms “infection” and “bacterial infection” may refer to acute otitis media. In one aspect, the terms “infection” and “bacterial infection” may refer to acute sinusitis. In another aspect, the terms “infection” and “bacterial infection” may refer to an infection caused by drug resistant bacteria. In still another aspect, the terms “infection” and “bacterial infection” may refer to catheter-related sepsis. In yet another aspect, the terms “infection” and “bacterial infection” may refer to chancroid. In a further aspect, the terms “infection” and “bacterial infection” may refer to chlamydia. In still a further aspect, the terms “infection” and “bacterial infection” may refer to community-acquired pneumonia (CAP). In yet a further aspect, the terms “infection” and “bacterial infection” may refer to complicated skin and skin structure infection. In one aspect, the terms “infection” and “bacterial infection” may refer to uncomplicated skin and skin structure infection. In another aspect, the terms “infection” and “bacterial infection” may refer to endocarditis. In still another aspect, the terms “infection” and “bacterial infection” may refer to febrile neutropenia. In yet another aspect, the terms “infection” and “bacterial infection” may refer to gonococcal cervicitis. In a further aspect, the terms “infection” and “bacterial infection” may refer to gonococcal urethritis. In still a further aspect, the terms “infection” and “bacterial infection” may refer to hospital-acquired pneumonia (HAP). In yet another aspect, the terms “infection” and “bacterial infection” may refer to osteomyelitis. In a further aspect, the terms “infection” and “bacterial infection” may refer to sepsis. In still a further aspect, the terms “infection” and “bacterial infection” may refer to syphilis.

In a further aspect, there is provided a pharmaceutical composition comprising a compound of Formula (I), or a pharmaceutically acceptable salt thereof, and at least one pharmaceutically acceptable carrier, diluent, or excipient.

In one aspect, there is provided a pharmaceutical composition comprising a compound of Formula (Ia), or a pharmaceutically acceptable salt thereof, and at least one pharmaceutically acceptable carrier, diluent, or excipient.

In one aspect, there is provided a pharmaceutical composition comprising a compound of Formula (Ib), or a pharmaceutically acceptable salt thereof, and at least one pharmaceutically acceptable carrier, diluent, or excipient.

In still a further aspect, there is provided a pharmaceutical composition comprising a compound of Formula (Ic), or a pharmaceutically acceptable salt thereof, and at least one pharmaceutically acceptable carrier, diluent, or excipient.

In yet a further aspect, there is provided a pharmaceutical composition comprising a compound of Formula (Id), or a pharmaceutically acceptable salt thereof, and at least one pharmaceutically acceptable carrier, diluent, or excipient.

In one aspect, there is provided a pharmaceutical composition comprising a compound of Formula (Ie), or a pharmaceutically acceptable salt thereof, and at least one pharmaceutically acceptable carrier, diluent, or excipient.

In one aspect, there is provided a pharmaceutical composition comprising a compound of Formula (If), or a pharmaceutically acceptable salt thereof, and at least one pharmaceutically acceptable carrier, diluent, or excipient.

In one aspect, there is provided a pharmaceutical composition comprising a compound of Formula (Ig), or a pharmaceutically acceptable salt thereof, and at least one pharmaceutically acceptable carrier, diluent, or excipient.

In one aspect, there is provided a pharmaceutical composition comprising a compound of Formula (Ih), or a pharmaceutically acceptable salt thereof, and at least one pharmaceutically acceptable carrier, diluent, or excipient.

In one aspect, there is provided a pharmaceutical composition comprising a compound of Formula (II), or a pharmaceutically acceptable salt thereof, and at least one pharmaceutically acceptable carrier, diluent, or excipient.

In one aspect, there is provided a pharmaceutical composition comprising a compound of Formula (IIa), or a pharmaceutically acceptable salt thereof, and at least one pharmaceutically acceptable carrier, diluent, or excipient.

In one aspect, there is provided a pharmaceutical composition comprising a compound of Formula (IIb), or a pharmaceutically acceptable salt thereof, and at least one pharmaceutically acceptable carrier, diluent, or excipient.

In one aspect, there is provided a pharmaceutical composition comprising a compound of Formula (IIc), or a pharmaceutically acceptable salt thereof, and at least one pharmaceutically acceptable carrier, diluent, or excipient.

In one aspect, there is provided a pharmaceutical composition comprising a compound of Formula (IIe), or a pharmaceutically acceptable salt thereof, and at least one pharmaceutically acceptable carrier, diluent, or excipient.

In one aspect, there is provided a pharmaceutical composition comprising a compound of Formula (IIf), or a pharmaceutically acceptable salt thereof, and at least one pharmaceutically acceptable carrier, diluent, or excipient.

In one aspect, there is provided a pharmaceutical composition comprising a compound of Formula (IIg), or a pharmaceutically acceptable salt thereof, and at least one pharmaceutically acceptable carrier, diluent, or excipient.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate; granulating and disintegrating agents such as corn starch or algenic acid; binding agents such as starch; lubricating agents such as magnesium stearate, stearic acid or talc; preservative agents such as ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form or in the form of nano or micronized particles together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives such as ethyl or propyl p-hydroxybenzoate; anti-oxidants such as ascorbic acid); coloring agents; flavoring agents; and/or sweetening agents such as sucrose, saccharine or aspartame.

Oily suspensions may be formulated by suspending the active ingredient in a vegetable oil such as arachis oil, olive oil, sesame oil or coconut oil or in a mineral oil such as liquid paraffin. The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 4 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

In addition to the compounds of the present invention, the pharmaceutical composition of this invention may also contain or be co-administered (simultaneously, sequentially or separately) with one or more known drugs selected from other clinically useful classes of antibacterial agents (for example, macrolides, quinolones, B-lactams or aminoglycosides) and/or other anti-infective agents (for example, an antifungal triazole or amphotericin). These may include carbapenems, for example meropenem or imipenem, to broaden the therapeutic effectiveness. Compounds of this invention may also contain or be co-administered with bactericidal/permeability-increasing protein (BPI) products or efflux pump inhibitors to improve activity against gram negative bacteria and bacteria resistant to antimicrobial agents.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. Accordingly, the optimum dosage may be determined by the practitioner who is treating any particular patient.

In addition to its use in therapeutic medicine, the compound of Formulas (I) and its pharmaceutically acceptable salts are also useful as pharmacological tools in the development and standardization of in vitro and in vivo test systems for the evaluation of antibacterial effects in laboratory animals such as cats, dogs, rabbits, monkeys, rats and mice, as part of the search for new therapeutic agents.

In any of the above-mentioned pharmaceutical composition, process, method, use, medicament, and manufacturing features of the instant invention, any of the alternate embodiments of the compounds of the invention described herein also apply.

### Process

If not commercially available, the necessary starting materials for the procedures such as those described herein may be made by procedures which are selected from standard organic chemical techniques, techniques which are analogous to the synthesis of known, structurally similar compounds, or techniques which are analogous to the described procedure or the procedures described in the Examples.

It is noted that many of the starting materials for synthetic methods as described herein are commercially available and/or widely reported in the scientific literature, or could be made from commercially available compounds using adaptations of processes reported in the scientific literature. The reader is further referred to Advanced Organic Chemistry, 5^{th} Edition, by Jerry March and Michael Smith, published by John Wiley & Sons 2001, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, published by John Wiley and Sons, 1991).

Examples of suitable protecting groups for a hydroxy group are, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric, phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid, for example boron tris(trifluoroacetate). A suitable alternative protecting group for a primary amino group is, for example, a phthaloyl group, which may be removed by treatment with an alkylamine, for example dimethylaminopropylamine or 2-hydroxyethylamine, or with hydrazine. Another suitable protecting group for an amine is, for example, a cyclic ether such as tetrahydrofuran, which may be removed by treatment with a suitable acid such as trifluoroacetic acid.

The protecting groups may be removed at any convenient stage in the synthesis using conventional techniques well known in the chemical art, or they may be removed during a later reaction step or work-up.

In one aspect, the present invention provides a process for preparing a compound of Formula (I) or Formula (II), or a pharmaceutically acceptable salt thereof, said process comprising reacting a compound of Formula (AA):

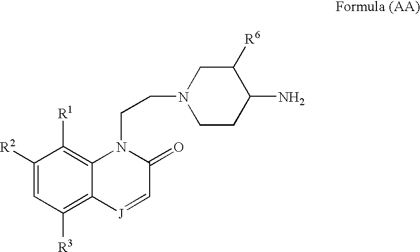

with a compound of Formula (AB):

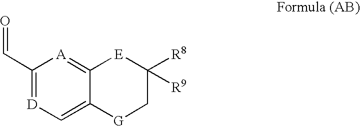

in the presence of a suitable reducing agent,

and thereafter if necessary:
i) converting the compound of Formula (I) or Formula (II) into another compound of Formula (I) or Formula (II);
ii) removing any protecting groups; and/or
iii) forming a pharmaceutically acceptable salt.

The compound of Formula (AA) may be reacted with the compound of Formula (AB) under typical reductive amination conditions. The first step of the reaction, the imine-formation, typically takes place in the presence of a dehydrating agent such as molecular sieves (MS 3 Å), though the reaction will generally proceed without a dehydrating agent. A suitable solvent is methanol or methanol/chloroform mixtures. The imine intermediate is typically not isolated; rather, the reducing agent is generally added to the reaction mixture after imine formation. Suitable reducing agents for the second (reduction) step of the process include boron reducing agents such as NaB(OAc)₃H or NaBH₃CN.

In another aspect, the present invention provides a process for preparing a compound of Formula (I) or (II) in which R⁶ is F, or a pharmaceutically acceptable salt thereof, said process comprising reacting a compound of Formula (AC):

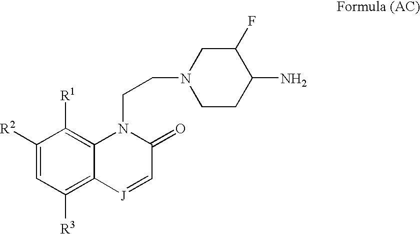

with a compound of Formula (AB):

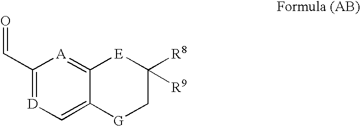

in the presence of a suitable reducing agent,

and thereafter if necessary:
i) converting the compound of Formula (I) or (II) into another compound of Formula (I) or (II);
ii) removing any protecting groups; and/or
iii) forming a pharmaceutically acceptable salt.

The reaction conditions for the reaction of a compound of Formula (AC) with a compound of Formula (AB) are as described above for the reaction of a compound of Formula (AA) with a compound of Formula (AB).

In still another aspect, the present invention provides a process for preparing a compound of Formula (I) or (II), or a pharmaceutically acceptable salt thereof, said process comprising reacting a compound of Formula (BI):

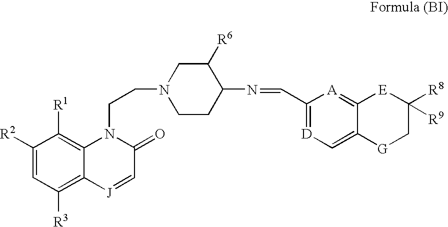

with a suitable reducing agent,

and thereafter if necessary:
i. converting the compound of Formula (I) or (II) into another compound of Formula (I) or (II);
ii. removing any protecting groups; and/or
iii. forming a pharmaceutically acceptable salt.

Suitable reducing agents for the reduction of the compound of Formula (BI) include boron reducing agents such as NaB(OAc)₃H or NaBH₃CN. A suitable solvent is methanol or methanol/chloroform mixtures

Compounds of Formula (AA) may be prepared as shown in Scheme 1.

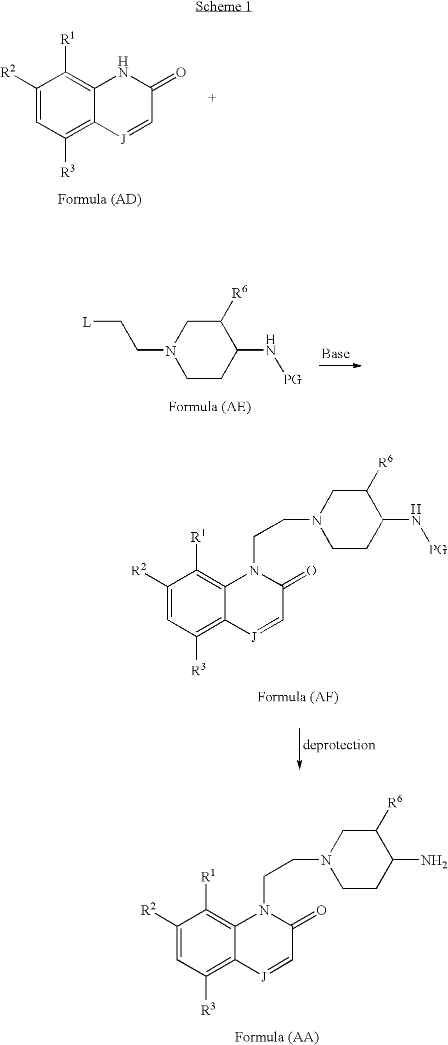

Reaction of a compound of Formula (AD) with a compound of Formula (AE) in the presence of a base such as sodium hydride may be used to obtain a compound of Formula (AF). Deprotection provides a compound of Formula (AA). Suitable leaving groups include leaving groups such as mesylate, chloro, bromo, and iodo. Suitable protecting groups include alkoxycarbonyl groups such as t-butoxycarbonyl, which may be deprotected using acids such as HCl.

Another method that may be used to prepare compounds of Formula (AA) is shown in Scheme 2.

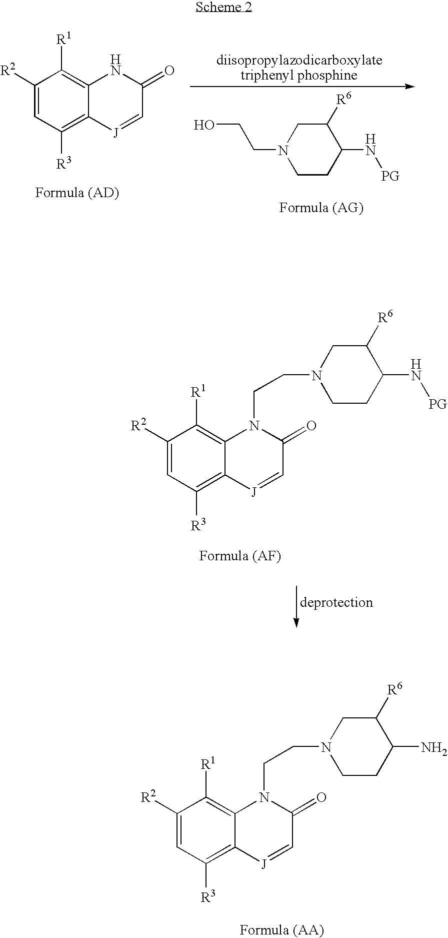

Reaction of a compound of Formula (AD) under Mitsunobu conditions with a compound of Formula (AG) may be used to obtain a compound of Formula (AF). Deprotection provides a compound of Formula (AA). Suitable protecting groups include alkoxycarbonyls such as t-butoxycarbonyl, which may be removed using acids such as HCl.

Scheme 3 depicts another method for preparing compounds of Formula (AA).

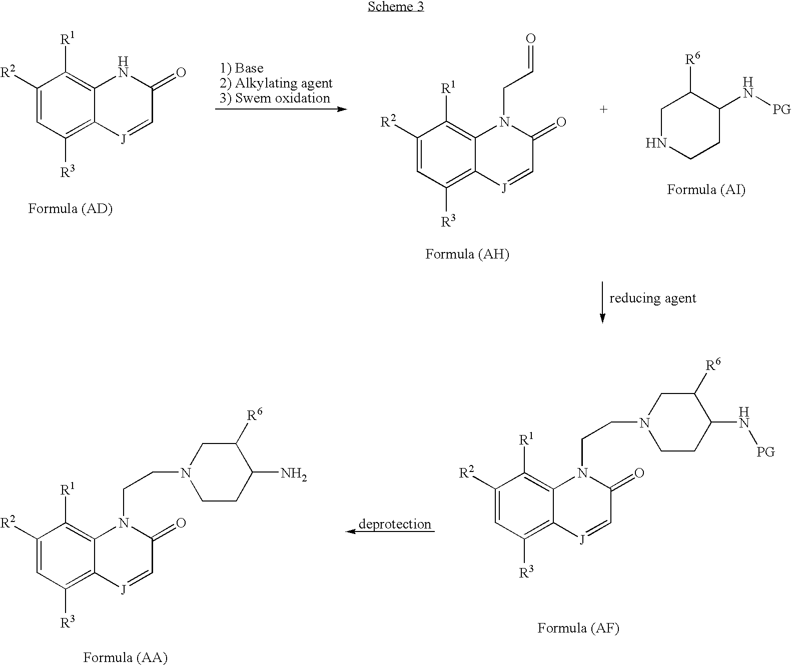

A compound of Formula (AD) may be reacted with a base such as NaH, followed by reaction with bromo- or chloroethanol or bromo-or chloro acetaldehyde, or with a protected derivative thereof, (followed by deprotection and oxidation in case of an alcohol derivative), providing a compound of Formula (AH). The compound of Formula (AH) may be reacted with the compound of Formula (AI) under typical reductive amination conditions. As noted above for the reaction of the compound of Formula (AA) with the compound of Formula (AB), the imine-formation typically takes place in the presence of a dehydrating agent such as molecular sieves (MS 3 Å), though the reaction will generally proceed just as well without a dehydrating agent. A suitable solvent is tetrahydrofuran, dichloromethane or chloroform/methanol mixtures. The imine intermediate is typically not isolated; rather, the reducing agent is generally added to the reaction mixture after imine formation. Suitable reducing agents for the second step of the reaction (the reduction) include boron reducing agents such as NaB(OAc)₃H or NaBH₃CN. Deprotection of the compound of Formula (AF) provides a compound of Formula (AA). Suitable protecting groups for the amino group substituent on the piperidine ring of the compound of Formula (AI) include alkoxycarbonyls such as t-butoxycarbonyl, which may be removed using acids such as HCl; and azides, which may be converted to the amine reductively, either with triphenyl phosphine (Staudinger Reaction) or by hydrogenation.

Compounds of Formula (AL), which are compounds of Formula (AE) in which the leaving group is a mesylate leaving group, may be prepared according to Scheme 4.

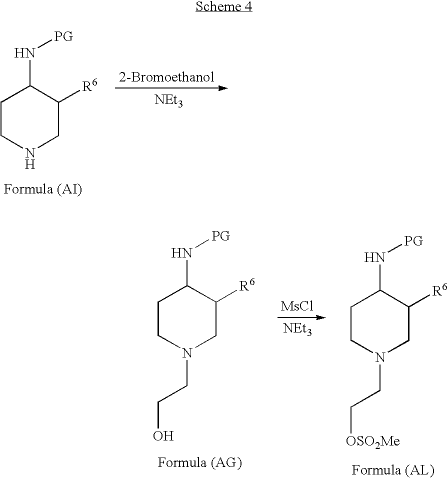

A compound of Formula (AL) may be prepared by reaction of a compound of Formula (AI) with bromoethanol or a derivative thereof in the presence of a base such as triethylamine, providing a compound of Formula (AG). The compound of Formula (AG) may be reacted with mesyl chloride, in the presence of a base, such as trialkyl amine or an immobilized version thereof on a resin, providing a compound of Formula (AL). The compound of Formula (AL) is potentially unstable, and may be present as part of a mixture with the corresponding chloride (arising from attack of chloride on the mesyl group) and needs to be prepared fresh under careful controlled conditions.

A compound of Formula (AO), which is a compound of Formula (AD) in which J is CH, may be prepared according to Scheme 5.

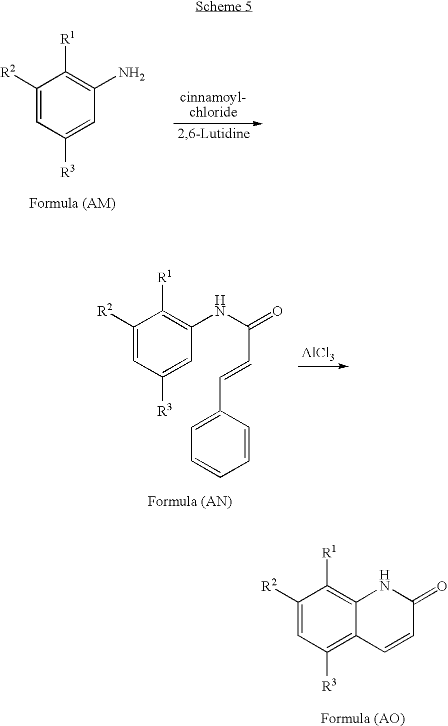

As shown in Scheme 5, a compound of Formula (AO) may be prepared from commercially available compounds of Formula (AM) by reaction with cinnamoylchloride in the presence of a suitable base such as 2,6-lutidine, followed by cyclization with aluminum trichloride.

In similar manner, cinnamoyl chloride may be replaced with E-ethoxyacryloyl chloride and cyclization of the intermediate E-ethoxyacryloylamides affected with sulfuric acid instead of aluminium trichloride (E. Baston et al, European Journal of Medicinal Chemistry 35 (2000) 931). Unsymmetrical substituted anilines generally lead to regioisomeric quinolin-2(1H)-one derivatives, which may be difficult to separate by chromatography due to limited solubility. Such mixtures may be separated either by crystallization or may be converted to the corresponding 2-chloroquinoline derivatives (for example with phosphorusIII oxychloride), which may be separated by chromatography or by crystallization and can then be hydrolyzed back with refluxing hydrochloride acid to the single regioisomers of the compound of Formula (AO).

A compound of Formula (AO) may also be prepared according to Scheme 6.

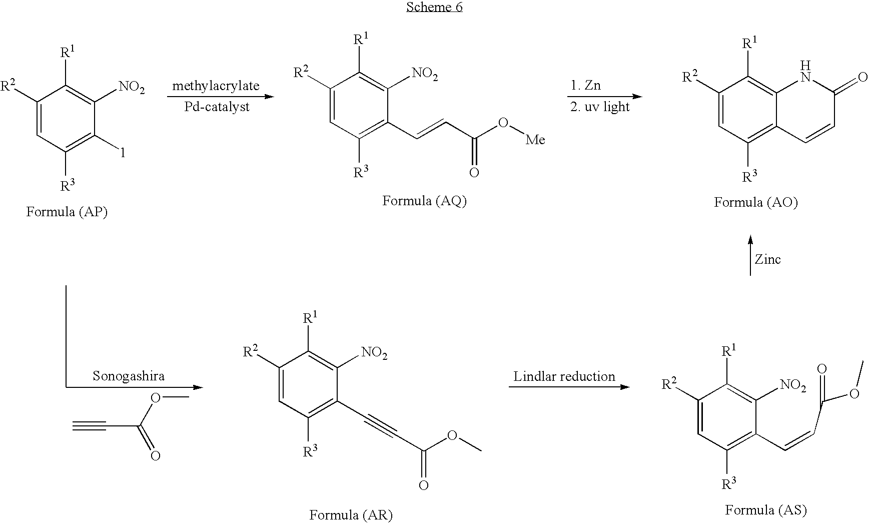

A compound of Formula (AO) may be prepared from a compound of Formula (AP) by forming a carbon-carbon bond, followed by intramolecular amide bond formation of an appropriately set up cis unsaturated system. The isomerization of a trans double bond may be performed thermically or photochemically under uv light. Alternatively, the carbon-carbon bond formation may be performed as a Sonogashira Coupling to an alkyne intermediate, which may be partially hydrogenated to a cis double bond under Lindlar conditions.

A compound of Formula (AO) may also be prepared according to Scheme 7.

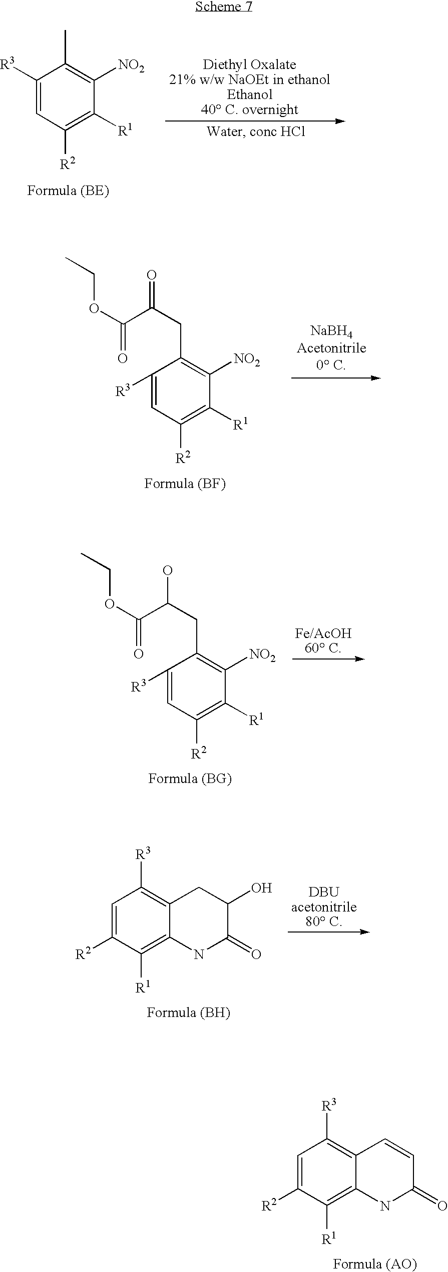

A compound of the Formula (AO) may be prepared from a compound of Formula (BE) by deprotonation with a base such as sodium ethoxide, followed by reaction with diethyl oxalate to form a compounds of the Formula (BF). This intermediate may then be reduced with a suitable reducing agent such as sodium borohydride, providing a compound of Formula (BG). The compound of Formula (BG) may then be cyclized to a compound of Formula (BH), after reduction of the nitro group with a suitable reducing agent such as iron in acetic acid or tin dichloride. Finally, elimination with a base such as DBU, provides a compound of Formula (AO).

A compound of Formula (AU), which is a compound of Formula (AD) in which J is N, may be prepared according to Scheme 8.

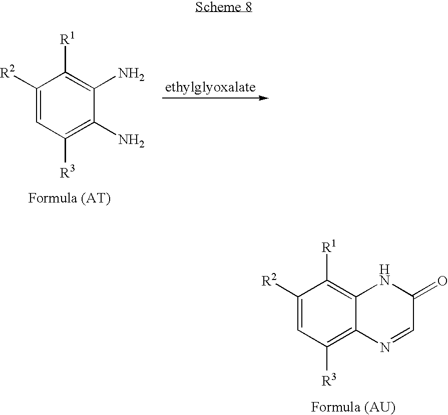

Regioisomeric mixtures of the compound of Formula (AU) may be separated by the methods described above for Scheme 5.

Scheme 9 depicts another method for preparing compound of Formula (AU).

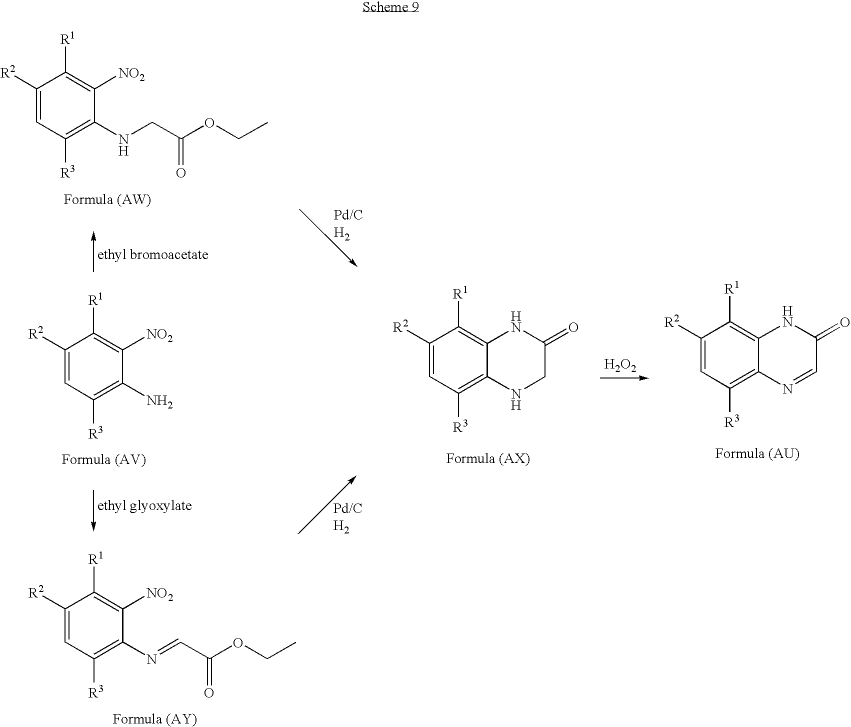

A compound of Formula (AU) may be obtained by oxidation of a compound of Formula (AX) with oxidants such as hydrogen peroxide. The compound of Formula (AX) may be obtained by reacting a compound a compound of Formula (AV) with bromoacetic acid esters, or with ethylgloxylate, followed by reduction of the nitro group and spontaneous cyclization. Suitable reducing agents include Pd/C and H₂, iron in acetic acid, and tin chloride.

A compound of Formula (AZ), which is a compound of Formula (AG) in which the protecting group is t-butoxycarbonyl, may be prepared according to scheme 10.

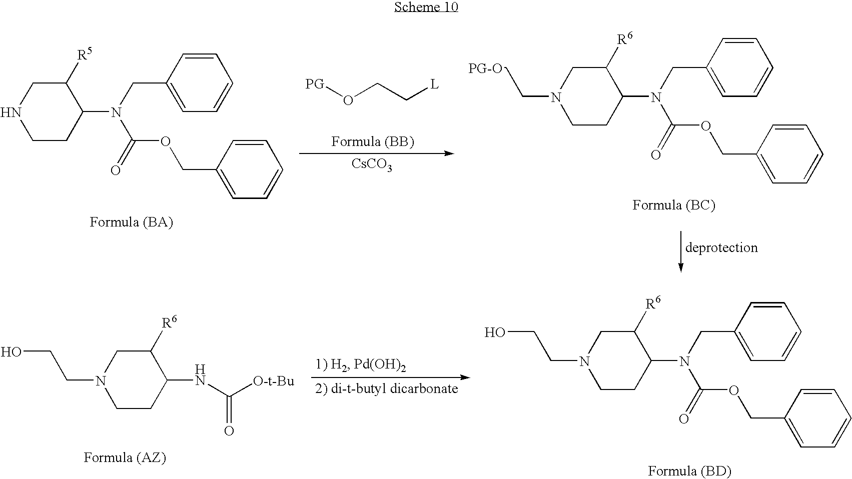

A compound of Formula (BA) may be reacted with a compound of Formula (BB) in the presence of a base providing a compound of Formula (BC). Examples of Cs₂CO₃, NaH, K₂CO₃ or Na₂CO₃. Suitable leaving groups for this reaction include mesylate and halo groups such as chloro and bromo. Suitable protecting groups for this reaction include silyl protecting groups such as t-butyl-dimethylsilyl. The protecting group of the compound of Formula (BC) may be removed using t-butylammonium fluoride, providing a compound of Formula (BD). The compound of Formula (BD) may be deprotected by hydrogenation. Examples of suitable catalysts for such a reaction include Pd(OH)₂, Platinum black, and PtO₂, followed by reaction with di-t-butylcarbonate, providing a compound of Formula (AZ).

When an optically active form of a compound of the invention is required, it may be obtained by carrying out one of the above procedures using a pure enantiomer as a starting material, or by resolution of a mixture of the enantiomers or diastereomers of the final products or chiral intermediates using a standard procedure. The resolution of enantiomers may be achieved by chromatography on a chiral stationary phase, such as a Chiralpak AD column. Consideration has to be given to solubility as well as resolution. Alternatively, resolution may be obtained by preparation and selective crystallization of a diastereomeric salt of a chiral intermediate or chiral product with a chiral acid, such as camphersulfonic acid. Alternatively, a method of stereoselective synthesis may be employed, for example by using a chiral variant of a protection group, a chiral catalyst or a chiral reagent where appropriate in the reaction sequence.

Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

The skilled organic chemist will be able to use and adapt the information contained and referenced within the above references, and accompanying Examples therein and also the Examples herein, to obtain necessary starting materials and products.

### Examples

The invention is now illustrated by but not limited to the following Examples, for which, unless otherwise stated:
(i) evaporations were carried out by rotary evaporation in vacuo and work-up procedures were carried out after removal of residual solids by filtration;
(ii) temperatures are quoted as ° C.; operations were carried out at room temperature, that is typically in the range 18-26° C. and without the exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) in general, the course of reactions was followed by TLC, HPLC, or LC/MS and reaction times are given for illustration only; yields are given for illustration only and are not necessarily the maximum attainable;
(v) the structure of the end-products of the invention were generally confirmed by NMR and mass spectral techniques. Proton magnetic resonance spectra were generally determined in DMSO-d₆ unless otherwise stated, using a Bruker DRX-300 spectrometer or a Bruker DRX-400 spectrometer, operating at a field strength of 300 MHz, or 400 MHz, respectively. In cases where the NMR spectrum is complex, only diagnostic signals are reported. Chemical shifts are reported in parts per million downfield from tetramethylsilane as an internal standard (δ scale) and peak multiplicities are shown thus: s, singlet; d, doublet; dd, doublet of doublets; dt, doublet of triplets; dm, doublet of multiplets; t, triplet, m, multiplet; br, broad. Fast-atom bombardment (FAB) mass spectral data were generally obtained using a Platform spectrometer (supplied by Micromass) run in electrospray and, where appropriate, either positive ion data or negative ion data were collected or using Agilent 1100 series LC/MSD equipped with Sedex 75ELSD, and where appropriate, either positive ion data or negative ion data were collected. The lowest mass major ion is reported for molecules where isotope splitting results in multiple mass spectral peaks (for example when chlorine is present). Reverse Phase HPLC was carried out using YMC Pack ODS-AQ (100×20 mmID, S-5μ particle size, 12 nm pore size) on Agilent instruments;
(vi) each intermediate was purified to the standard required for the subsequent stage and was characterized in sufficient detail to confirm that the assigned structure was correct; purity was assessed by HPLC, TLC, or NMR and identity was determined by infra-red spectroscopy (IR), mass spectroscopy or NMR spectroscopy as appropriate; and
(vii) the following abbreviations may be used:
   TLC is thin layer chromatography; HPLC is high pressure liquid chromatography; MPLC is medium pressure liquid chromatography; NMR is nuclear magnetic resonance spectroscopy; DMSO is dimethylsulfoxide; CDCl₃ is deuterated chloroform; Me0D is deuterated methanol, i.e. D₃COD; MS is mass spectroscopy; ESP (or ES) is electrospray; EI is electron impact; APCI is atmospheric pressure chemical ionization; THF is tetrahydrofuran; DCM is dichloromethane; MeOH is methanol; DMF is dimethylformamide; EtOAc is ethyl acetate; LC/MS is liquid chromatography/mass spectrometry; h is hour(s); min is minute(s); d is day(s); MTBD is N-methyl-1,5,7-triazabicyclo[4.4.0]dec-5-ene; TFA is trifluoroacetic acid; v/v is ratio of volume/volume; Boc denotes t-butoxycarbonyl; Cbz denotes benzyloxycarbonyl; Bz denotes benzoyl; atm denotes atmospheric pressure; rt denotes room temperature; mg denotes milligram; g denotes gram; pi, denotes microliter; mL denotes milliliter; L denotes liter; 1 μM denotes micromolar; mM denotes millimolar; M denotes molar; N denotes normal; nm denotes nanometer.

### Intermediate 1

### 1-{2-[(3R,4S)-4-Amino-3-hydroxypiperidin-1-yl]ethyl}-2-oxo-1,2-dihydroquinoline-7-carbonitrile

and

### Intermediate 2

### 1-{2-[(3S,4R)-4-Amino-3-hydroxypiperidin-1-yl]ethyl}-2-oxo-1,2-dihydroquinoline-7-carbonitrile

A mixture of 1-[2-cis(±) (4-azido-3-hydroxypiperidin-1-yl)ethyl]-2-oxo-1,2-dihydroquinoline-7-carbonitrile (Intermediate 3) (0.545 g, 1.61 mmol) and triphenylphophine (0.507 g, 1.93 mmol) in acetonitrile/water (9:1, 50 mL) was stirred at room temperature for 6 days. The reaction mixture was concentrated to dryness under reduced pressure. Chromatography on silica gel with dichloromethane/methanol (6:1, containing 0.2% ammonium hydroxide) gave the racemic mixture of Intermediate 1 and 2 as a colorless hard foam, 0.452 g (90%).

MS (ESP): 313 (MH⁺) for C₁₇H₂₀N₄O₂

¹H-NMR (DMSO-4) δ: 1.48 (m, 2H); 2.20-2.40 (m, 2H); 2.45-2.61 (m, 4H); 2.72 (m, 1H); 3.44 (m, 1H); 4.35 (dd, 2H); 6.78 (d, 1H); 7.64 (d, 1H); 7.90 (d, 1H); 8.00 (d, 1H); 8.08 (d, 1H). (OH and NH₂ protons were exchanged with methanol).

The racemic mixture was separated on a Chiralpak AD column (250×20 mm, 10 micron) with 60% hexanes and 40% ethanol/methanol (1:1), containing 0.1% diethyl amine. Intermediate 2 was eluting first, [α]_{D}=+45.5, followed by Intermediate 1, [α]_{D}=−45.9 (in methanol/chloroform 1:1, c=1).

### Intermediate 3

### 1-[2-Cis(±) (4-azido-3-hydroxypiperidin-1-yl)ethyl]-2-oxo-1,2-dihydroquinoline-7-carbonitrile

A solution of 1-[2-cis±(4-azido-3-{[tert-butyl(dimethypsilyl]oxy}piperidin-1-yl)ethyl]-2-oxo-1,2-dihydroquinoline-7-carbonitrile (Intermediate 4) (0.724 g, 1.6 mmol) in THF (5 mL) was treated dropwise at room temperature with a solution of tetrabutyl ammonium fluoride in THF (1M, 2.2 mL). After one hour saturated aqueous sodium hydrogencarbonate solution (10 mL) was added and THF was removed under reduced pressure. It was extracted with dichloromethane/ether (1:1, ˜200 mL). The organic phase, containing some insoluble product was separated, the aqueous phase was back extracted with dichloromethane (100 mL) and the combined organic phases were concentrated to dryness under reduced pressure and dried under high vacuum to give the product as a colorless solid, 0.545 g (quant.).

¹H-NMR (DMSO-d₆) δ: 1.56 (m, 1H); 1.71 (m, 1H); 2.25-2.63 (m, 6H); 3.67 (m, 2H); 4.35 (dd, 2H); 5.06 (m, 1H); 6.78 (d, 1H); 7.64 (dd, 1H); 7.90 (d, 1H); 8.00 (d, 1H); 8.08 (brs, 1H).

### Intermediate 4

### 1-[2-Cis(±) (4-azido-3-{[tert-butyl(dimethynsilyl]oxy}piperidin-1-yl)ethyl]-2-oxo-1,2-dihydroquinoline-7-carbonitrile

A suspension of 2-oxo-1,2-dihydroquinoline-7-carbonitrile (Intermediate 5) (0.51 g, 3.0 mmol) in DMF (15 mL) was treated at 0° C. with sodium hydride (in oil, 60%, 132 mg). It was stirred for 2 hours, then a solution of 2-cis±(4-azido-3-{[tert-butyl(dimethyl)silyl]oxy}piperidin-1-yl)ethyl methanesulfonate (Intermediate 6) (3 mmol) in DMF (7 mL) was added via syringe. The cooling was removed and it was stirred over night at room temperature. DMF was removed under reduced pressure, the residue was taken up in dichloromethane (˜200 mL) and saturated aqueous sodium hydrogencarbonate solution (20 mL, pH adjusted to pH10 with 1M NaOH solution). The aqueous phase was back-extracted two times with dichloromethane (2×100 mL) and the combined organic phases were dried over sodium sulfate. Chromatography on silica gel with hexanes/acetone (4:1) gave the product as a hard foam, 0.724 g (53%).

MS (ESP): 453 (MH⁺) for C₂₃H₃₂N₆O₂Si

¹H-NMR (DMSO-d₆) δ: 0.00 and 0.03 (2xs, 6H); 0.80 (s, 9H); 1.59 (m, 1H); 1.70 (m, 1H); 2.30-2.65 (m, 6H); 3.69 (m, 1H); 3.80 (m, 1H); 4.29 (m, 1H); 4.42 (m,1H); 6.77 (d, 1H); 7.63 (d, 1H); 7.90 (d, 1H); 7.99 (d, 1H); 8.08 (s, 1H).

### Intermediate 5

### 2-Oxo-1,2-dihydroquinoline-7-carbonitrile

7-Bromoquinolin-2(1H)-one (Intermediate 46) (9.21 g, 41 mmol) and copper (I) cyanide (4.05 g) were heated in N-methylpyrrolidone (50 mL) at 160° C. for 16 hours. It was cooled to room temperature, an aqueous solution of ethylenediamine tetra acetate (2M, pH 8.3, 100 mL) was added and the mixture was stirred at room temperature and open to air for 5 days. The precipitate was collected by filtration through a 0.45 μm membrane, washed with water and ethyl acetate and recrystallized from DMF/water to give the product as a brown solid, 90% purity, which was used without further purification, 4.72 g (60%).

MS (ESP): 171 (MH⁺) for C₁₀H₆N₂O

¹H-NMR (DMSO-d₆) δ: 6.66 (d, 1H); 7.55 (d, 1H); 7.61 (s, 1H); 7.85 (d, 1H); 7.99 (d, 1H); 12.01 (s, 1H).

### Alternate Procedure for the Synthesis of Intermediate 5

A mixture of 3-hydroxy-2-oxo-1,2,3,4-tetrahydroquinoline-7-carbonitrile (Intermediate 66, 15.51 g, 82.42 mmol) and 1,8-diazabicyclo[5.4.0]undec-7ene (DBU) in acetonitrile (155 mL) was heated at 75° C. for 2.5 hours. The reaction mixture was cooled to room temperature and a precipitate was collected by filtration, washed with water (77 mL) and with methanol (77 mL) and dried under reduced pressure to give the product as an off-white solid, 9.71 g (68%).

Mp>250° C.

MS (ESP): 171 (MH⁺) for C₁₀H₆N₂O

¹H-NMR (DMSO-d₆) δ ppm 6.67 (d, 1H); 7.48-7.68 (m, 2H); 7.85 (d, 1H); 7.98 (d, 1H); 12.01 (s, 1H).

### Intermediate 6

### 2-(Cis(±))-(4-Azido-3-{[tert-butyl(dimethyl)silyl]oxy}piperidin-1-yl)ethyl methanesulfonate

A mixture of 2-(cis±)-(4-azido-3-{[tert-butyl(dimethyl)silyl]oxy}piperidin-1-yl)ethanol (Intermediate 7, 1.8 g, 6 mmol) in dry dichloromethane (20 mL) and triethyl amine (1.18 mL, 8.4 mmol) was treated at 0° C. with methanesulfonyl chloride (0.556 mL, 7.2 mmol). After 90 minutes the reaction was complete by TLC (chloroform/methanol 6:1, rf ˜0.9). Potassium phosphate buffer (pH 7, 1M, 15 mL) was added, dichloromethane was removed under reduced pressure and it was extracted with ice-cold ether (100 mL). The aqueous phase was back-extracted once with ether (50 mL) and the combined organic phases were dried over sodium sulfate. The solvent was removed under reduced pressure and the residue was taken up in DMF (10 mL). This crude preparation of the mesylate was used without delay for the next step.

MS (ESP): 379 (MH⁺) for C₁₄H₃₆N₄O₄SSi

### Intermediate 7

### 2-(Cis(±))-(4-Azido-3-{[tert-butyl(dimethyl)silyl]oxy}piperidin-1-yl)ethanol

A mixture of (cis±)-4-azido-3-{[tent-butyl(dimethypsilyl]oxy}piperidine (Intermediate 8) (1.625 g, 6.34 mmol), N,N-diisopropylethylamine (1.65 mL, 9.5 mmol and 2-bromoethanol (0.584 mL, 8.25 mmol) in dry acetonitrile (17 mL) was heated in the microwave at 70° C. for two hours. The solvent was removed under reduced pressure, the residue taken up in ethyl acetate (˜150 mL) and washed with saturated aqueous sodium hydrogencarbonate solution (˜25 mL). The aqueous phase was back-extracted once with ethyl acetate (100 mL) and the combined organic phases were dried over sodium sulfate. Chromatography on silica gel with dichloromethane/methanol (20:1) gave 1.80 g (95%) of product as a colorless oil.

MS (ESP): 301 (MH⁺) for C₁₃H₂₈N₄O₂Si

¹H-NMR (DMSO-d₆) δ: 0.08 (s, 6H); 0.87 (s, 9H); 1.65 (m, 2H); 2.18 (m, 1H); 2.25-2.60 (m, 5H); 3.44 (m, 2H); 3.73 (m, 1H); 3.91 (m, 1H); 4.35 (m, 1H).

### Intermediate 8

### (Cis(±))-4-azido-3-{[tert-butyl(dimethyl)silyl]oxy}piperidine

A solution of tert-butyl (cis±)-4-azido-3-{[tert-butyl(dimethypsilyl]oxy}piperidine-1-carboxylate (Intermediate 9) (2.3 g, 6.45 mmol) in dichloromethane (50 mL) was treated at 0° C. with trifluoroacetic acid (5 mL). After 3 hours the mixture was concentrated under reduced pressure and the residue was codistilled twice with dichloromethane. The residue was taken up in dichloromethane (100 mL) and washed with saturated aqueous sodium hydrogencarbonate solution (30 mL). The aqueous phase was back extracted once with dichloromethane (100 mL) and the combined organic phases were dried over sodium sulfate to give the product as a slightly yellow oil, 1.625 g (98%).

¹H-NMR (DMSO-d₆) δ: 0.07 and 0.09 (2xs, 6H); 0.88 (s, 9H); 1.49-1.73 (m, 2H); 2.45 (m, 1H); 2.56-2.69 (m, 3H); 3.65 (m, 1H); 3.79 (m, 1H).

### Intermediate 9

### tert-Butyl(cis(±))-4-azido-3-{[tert-butyl(dimethyl)silyl]oxy}piperidine-1-carboxylate

A mixture of tert-butyl(cis±)-4-azido-3-hydroxypiperidine-1-carboxylate (Intermediate 10) (1.76 g, 7.25 mmol) and imidazole (0.74 g, 10.9 mmol) in DMF (7 mL) at 0° C. was treated with tert-butyl dimethylsilyl chloride (1.3 g, 8.7 mmol). Cooling was removed and the mixture was stirred over night at room temperature. It was cooled to 0° C. and quenched with phosphate buffer (1M, pH 7, 20 mL). After 15 minutes, the mixture was diluted with ethyl acetate (100 mL), the organic phase was washed with water (2×50 mL) and dried over sodium sulfate. Chromatography on silica gel with hexanes/ethyl acetate (9:1) gave the product as a colorless oil, 2.3 g (89%).

¹H-NMR (DMSO-d₆) δ: 0.10 (s, 6H); 0.87 (s, 9H); 1.37 (s, 9H); 1.56-1.80 (m, 2H); 3.09-3.30 (m, 2H); 3.46 (m, 2H); 3.62 (m, 1H); 3.88 (m, 1H).

### Intermediate 10

### tert-Butyl (cis(±))-4-azido-3-hydroxypiperidine-1-carboxylate

To a mixture of (cis±)-4-azidopiperidin-3-ol (prepared following the procedure described in WO 2005/066176 for the chiral material) (2.1 g, 14.8 mmol) and potassium hydroxide (2.5 g, 44 mmol) in isopropanol (20 mL) and dichloromethane (25 mL) was added at 0° C. a solution of di-tert-butyl dicarbonate (3.9 g, 17.7 mmol) in dichloromethane (10 mL). The cooling was removed and it was stirred 2 hours at room temperature. It was quenched with water (50 mL) and isopropanol and dichloromethane were removed under reduced pressure. It was neutralized with potassium phosphate buffer (1M, pH 7, 100 mL), extracted with ethyl acetate twice (2×300 mL) and the combined organic phases were dried over sodium sulfate. Solvent was removed under reduced pressure and the residue was titurated from hexanes (˜20 mL) to give 0.966 g of product as a colorless solid. Chromatography of the mother liquors with hexanes/ethyl acetate (5:1) afforded 0.353 g of product (35%).

MS (ESP): 265.2 (MNa⁺) for C₁₀H₁₈N₄O₃

¹H-NMR (DMSO-d₆) δ: 1.39 (s, 9H); 1.58 (m, 1H); 1.74 (m, 1H); 3.20-3.40 (m, 4H); 3.69 (m, 2H); 5.40 (d, 1H).

### Intermediate 11

### 1-{2-[(3R,4S)-4-Amino-3-hydroxypiperidin-1-yl]ethyl}-7-methoxyquinoxalin-2(1H)-one

and

### Intermediate 12

### 1-{2-[(3S,4R)-4-Amino-3-hydroxypiperidin-1-yl]ethyl}-7-methoxyquinoxalin-2(1H)-one

A mixture of 1-[2-{(cis±)-(4-azido-3-hydroxypiperidin-1-yl)}ethyl]-7-methoxyquinoxalin-2(1H)-one (Intermediate 13) (0.507 g, 1.47 mmol) and triphenylphophine (0.463 g, 1.77 mmol) in acetonitrile/water (9:1, 20 mL) was stirred at room temperature for 5 days. The reaction mixture was concentrated to dryness under reduced pressure. The residue was taken up in dichloromethane (5 mL) and chromatographed on silica gel with dichloromethane/methanol (6:1, containing 0.2% ammonium hydroxide) to give the racemic mixture of Intermediate 11 and 12 as a colorless hard foam (0.422 g, 90%).

MS (ESP): 319 (MH⁺) for C₁₆H₂₂N₄O₃

¹H-NMR (DMSO-d₆) δ: 1.48 (m, 2H); 1.89 (m, 1H); 2.28 (m, 1H); 2.37 (dd, 1H); 2.54-2.62 (m, 3H); 2.71 (m, 1H); 3.45 (m, 1H); 3.91 (s, 3H); 4.30 (dd, 2H); 6.96-7.00 (m, 2H); 7.73 (d, 1H); 8.03 (s, 1H). (OH and NH₂ protons were exchanged with methanol).

The racemic mixture was separated on a Chiralpak AD column (250×20 mm, 10 micron) with ethanol/methanol (1:1), containing 0.1% diethyl amine. Intermediate 12 was eluting first, [α]_{D}=+45.5, followed by Intermediate 11, [α]_{D}=−44.7 (in methanol/chloroform 1:1, c=1).

### Intermediate 13

### 1-[2-{(Cis(±))-(4-azido-3-hydroxypiperidin-1-yl)}ethyl]-7-methoxyquinoxalin-2(1H)-one

A solution of 1-[2-(cis±)-4-azido-3-{[tert-butyl(dimethyl)silyl]oxy}piperidin-1-yl)ethyl]-7-methoxyquinoxalin-2(1H)-one (Intermediate 14, 0.721 g, 1.57 mmol) in THF (5 mL) was treated dropwise at room temperature with a solution of tetrabutyl ammonium fluoride in THF (1M, 2.2 mL). After one hour saturated aqueous sodium hydrogencarbonate solution (10 mL) was added and THF was removed under reduced pressure. It was extracted with dichloromethane/ether (1:1, ˜200 mL). The aqueous phase was back extracted with dichloromethane (100 mL) and the combined organic phases were dried over sodium sulfate. Chromatography on silica gel with hexanes/acetone (1:1) gave the product as a colorless hard foam, 0.507 g (94%).

MS (ESP): 345 (MH⁺) for C₁₆H₂₀N₆O₃

¹H-NMR (DMSO-d₆) δ: 1.58 (m, 1H); 1.70 (m, 1H); 2.25-2.65 (m, 6H); 3.67 (m, 2H); 3.90 (s, 3H); 4.31 (dd, 2H); 5.11 (m, 1H); 6.97-7.00 (m, 2H); 7.73 (d, 1H); 8.03 (s, 1H).

### Intermediate 14

### 1-[2-(Cis(±))-4-azido-3-{[tert-butyl(dimethyl)silyl]oxy}piperidin-1-yl)ethyl]-7-methoxyquinoxalin-2(1H)-one

7-Methoxyquinoxalin-2(1H)-one (Intermediate 15, 0.528 g, 3.0 mmol) was treated with sodium hydride (in oil, 60%, 132 mg) and 2-cis+(4-azido-3-{[tert-butyl(dimethyl)silyl]oxy}piperidin-1-yl)ethyl methanesulfonate (Intermediate 6, 3 mmol) using a procedure similar to the one described for the synthesis of Intermediate 4 to give the product as a hard foam, 0.721 g (52%).

MS (ESP): 459 (MH⁺) for C₂₂H₃₄N₆O₃Si

¹H-NMR DMSO-d₆) δ: 0.03 and 0.05 (2xs, 6H); 0.82 (s, 9H); 1.65 (m, 2H); 2.25-2.70 (m, 6H); 3.70 (m, 1H); 3.90 (s, 3H); 3.83 (m, 1H); 4.24 (m, 1H); 4.39 (m, 1H); 6.96-7.00 (m, 2H); 7.73 (m, 1H); 8.02 (s, 1H).

### Intermediate 15

### 7-Methoxyquinoxalin-2(1H)-one

To a solution of 8% aqueous sodium hydroxide (1.32 L) was added 7-methoxy-3,4-dihydroquinoxalin-2(1H)-one (Intermediate 16, 100 g) followed by a solution of 3 wt % hydrogen peroxide in water (1.17 L). The reaction mixture was slowly heated to 80° C. and maintained at this temperature for 4 hours. Then the heating source was removed and acetic acid (150 mL) was added dropwise. The suspension was stirred overnight at room temperature and the precipitated solid was collected by filtration to afford the product as a tan solid (90 g).

MS (ESP): 177 (MH⁺) for C₉H₈N₂O₂

¹H-NMR (DMSO-d₆) δ: 3.83 (s, 3H); 6.76 (d, 1H); 6.90 (dd, 1H); 7.67 (d, 1H); 7.97 (s, 1H); 12.32 (brs, 1H).

### Intermediate 16

### 7-Methoxy-3,4-dihydroquinoxalin-2(1H)-one

In a 18-L Parr apparatus a suspension of ethyl [(4-methoxy-2-nitrophenyl)imino]acetate (Intermediate 17) was hydrogenated at 55 psi in the presence of 20 wt % Pd/C (100 g, containing ˜50 wt % water) until no hydrogen was consumed. (Note: The reaction was strongly exothermic and the temperature should be controlled at around 60° C. by adjusting the rate of recharging hydrogen and by a cooling system). The reaction mixture was discharged, filtered over a celite cake, and evaporated under reduced pressure to give a crude solid that was triturated with MTBE (6 L) to give the product as a tan solid (400 g).

MS (ESP): 179 (MH⁺) for C₉H₁₀N₂O₂

¹H-NMR (DMSO-d₆) δ: 3.61 (m, 5H); 5.57 (m, 1H); 6.35-6.40 (m, 2H); 6.60 (m, 1H); 10.13 (brs, 1H).

### Alternative Procedure for Intermediate 16

Ethyl N-(4-methoxy-2-nitrophenyl)glycinate (Intermediate 18, 15.8 g crude) was taken up in 200 mL of 1:1 methanol/acetic acid, treated with 10% palladium on carbon (2 g), and stirred in an atmosphere of hydrogen overnight. The reaction mixture was filtered through celite and the filtrate was concentrated to dryness giving 10.6 g of the crude product as a tan solid. This was used without further purification.

MS (ESP): 179 (MH⁺) for C₉H₁₀N₂O₂

### Intermediate 17

### Ethyl [(4-methoxy-2-nitrophenyl)imino]acetate

A solution of 4-methoxy-2-nitroaniline (1 kg, 5.95 mol) and ethyl glyoxylate (1180 mL, 50 wt % in toluene, 5.95 mol) in toluene (10 L) was refluxed in a Dean-Stark apparatus for 48 hours and evaporated under reduced pressure to give the crude product as a dark brown oil, which was used without further purification.

### Intermediate 18

### Ethyl N-(4-methoxy-2-nitrophenyl)glycinate

A mixture of 4-methoxy-2-nitroaniline (25.0 g, 0.15 mol), ethyl bromoacetate (200 mL, 1.8 mol) and potassium carbonate (31.1 g, 0.23 mol) was heated at 150° C. for 4.5 hours. After cooling to room temperature aqueous sodium hydroxide solution (1M, 600 mL) was added. This mixture was extracted with ethyl acetate (2×500 mL). The combined organic phases were dried over magnesium sulfate and concentrated to dryness. Chromatography on silica gel with 25-50% acetone in hexanes gave 22.1 g of the crude product as a red solid. ¹H NMR revealed the presence of ˜20% dialkylated product. This material was used without further purification.

¹H NMR DMSO-d₆) δ: 1.18-1.23 (t, 3H); 3.74 (s, 3H); 4.12-4.18 (q, 2H); 4.23-4.25 (d, 2H); 6.90-6.93 (d, 1H); 7.25-7.29 (dd, 1H); 7.51-7.52 (d, 1H); 8.23-8.27 (t, 1H).

### Intermediate 19

### Cis(±) 1-[2-(4-amino-3-fluoropiperidin-1-yl)ethyl]-2-oxo-1,2-dihydroquinoline-7-carbonitrile

A solution of cis(±)tert-butyl {1-[2-(7-cyano-2-oxoquinolin-1(2H)-yl)ethyl]-3-fluoropiperidin-4-yl}carbamate (Intermediate 20, 397 mg, 0.95 mmol) in chloroform (8 mL) was treated at 0° C. with 30% trifluoroacetic acid in chloroform (5 mL). After 5 hours at room temperature the solvent was removed under reduced pressure to give the trifluoroacetate salt of the product, which was taken to the next step without further purification.

MS (ESP): 315 (MH⁺) for C₁₇H₁₉FN₄O

### Intermediate 20

### Cis(±)tert-butyl {1-[2-(7-cyano-2-oxoquinolin-1(2H)-yl)ethyl]-3-fluoropiperidin-4-yl}carbamate

A suspension of 2-oxo-1,2-dihydroquinoline-7-carbonitrile (Intermediate 5, 0.3 g, 1.7 mmol) in DMF (10 mL) was treated at room temperature with sodium hydride and cis(±) 2-{4-[(tert-butoxycarbonyl)amino]-3-fluoropiperidin-1-yl}ethyl methanesulfonate (Intermediate 21, ˜1.7 mmol) as described for Intermediate 4. Chromatography on silica gel with hexanes/ethyl acetate (2:3), gave the product as a solid, 397 mg (73%).

MS (ESP): 415 (MH⁺) for C₂₂H₂₇FN₄O₃

¹H-NMR (DMSO-d₆) δ: 1.37 (s, 9H); 1.48 (m, 1H); 1.67 (m, 1H); 2.27 (m, 2H); 2.56 (m, 2H); 2.96 (m, 1H); 3.15 (m, 1H); 3.46 (m, 1H); 4.34 (m, 2H); 4.60 (m, 1H); 6.77 (m, 1H); 6.91 (m, 1H); 7.64 (m, 1H); 7.90 (m, 1H); 7.99 (m, 1H); 8.07 (s, 1H).

### Intermediate 21

### Cis(±) 2-{4-[(tert-butoxycarbonyl)amino]-3-fluoropiperidin-1-yl}ethyl methanesulfonate

Cis(±)tert-butyl [3-fluoro-1-(2-hydroxyethyl)piperidin-4-yl]carbamate (Intermediate 22, 314 mg, 1.2 mmol) was reacted with mesyl chloride in the presence of triethyl amine using a procedure similar to the one described for the synthesis of Intermediate 6. The crude mesylate was used without delay for the next step.

### Intermediate 22

### Cis(±)tert-butyl[3-fluoro-1-(2-hydroxyethyl)piperidin-4-yl]carbamate

To a solution of cis(±)tert-butyl [1-(2-{[tert-butyl(dimethyl)silyl]oxy}ethyl)-3-fluoropiperidin-4-yl]carbamate (Intermediate 23, 530 mg, 1.4 mmol) in tetrahydrofuran (10 mL) at 0° C. was added tetrabutylammonium fluoride (1M in THF, 2.8 mL). After 30 minutes the reaction was quenched with saturated sodium bicarbonate and extracted twice with ethyl acetate, dried over magnesium sulfate and concentrated. Silica gel chromatography with 2.5% methanol in ethyl acetate afforded the product as a colorless solid, 314 mg (85%).

¹H-NMR (CDCl₃-d) δ: 1.43 (s, 9H); 1.81 (m, 2H); 2.30 (m, 1H); 2.36 (m, 1H); 2.59 (m, 2H); 2.75 (m, 1H); 2.95 (m, 1H); 3.24 (m, 1H); 3.61 (m, 2H); 3.71 (m, 1H); 4.68 (m, 1H); 4.85 (m, 1H).

### Intermediate 23

### Cis(±)tert-butyl [1-(2-{[tert-butyl(dimethyl)silyl]oxy}ethyl)-3-fluoropiperidin-4-yl]carbamate

Cis(±) 1-(2-{[tert-butyl(dimethyl)silyl]oxy}ethyl)-3-fluoropiperidin-4-amine (Intermediate 24, 2.8 g, 10.4 mmol) and di-tert-butyl dicarbonate (3.4 g, 15.6 mmol) were combined in tetrahydrofuran (50 mL) at room temperature. After 90 minutes the reaction mixture was concentrated under reduced pressure. Silica gel chromatography with hexanes/ethyl acetate (3:2) afforded the product as a colorless oil, 3.2 g (82%).

¹H-NMR (CDCl₃-d) δ: 0.03 (s, 6H); 0.86 (s, 9H); 1.43 (s, 9H); 1.77 (m, 2H); 2.25 (m, 1H); 2.37 (m, 1H); 2.58 (m, 2H); 2.95 (m, 1H); 3.26 (m, 1H); 3.62 (m, 1H); 3.74 (m, 2H); 4.65 (m, 1H); 4.83 (m, 1H).

### Intermediate 24

### Cis(±) 1-(2-{[tert-butyl(dimethyl)silyl]oxy}ethyl)-3-fluoropiperidin-4-amine

Cis(±) benzyl benzyl[1-(2-{[tert-butyl(dimethyl)silyl]oxy}ethyl)-3-fluoropiperidin-4-yl]carbamate (Intermediate 25, 5.2 g, 10.4 mmol) was hydrogenated in anhydrous methanol (15 mL) on palladium hydroxide 20 wt. % on carbon (31 mg) for 24 hours, then filtered through celite and concentrated under reduced pressure to give the product as a colorless oil, 2.8 g.

### Intermediate 25

### Cis(±) benzyl benzyl[1-(2-{[tert-butyl(dimethyl)silyl]oxy}ethyl)-3-fluoropiperidin-4-yl]carbamate

A mixture of cis(±)benzyl benzyl(3-fluoropiperidin-4-yl)carbamate hydrochloride (Intermediate 26, 4.3 g, 6.1 mmol), (2-bromoethoxy)-tert-butyldimethylsilane (9.8 mL, 45.7 mmol) and cesium carbonate (9.9 g, 30.4 mmol) in acetonitrile (150 mL) was heated at 60° C. overnight. The reaction mixture was filtered and concentrated under reduced pressure. Silica gel chromatography with hexanes/ethyl acetate (3:2) afforded the product as a colorless oil, 5.2 g (91%).

MS (ESP): 501 (MH⁺) for C₂₈H₄FN₂O₃Si

### Intermediate 26

### Cis(±)benzyl benzyl(3-fluoropiperidin-4-yl)carbamate

To a solution of cis(±)tert-butyl 4-{benzyl[(benzyloxy)carbonyl]amino}-3-fluoropiperidine-1-carboxylate (Intermediate 27, 6 g, 13.5 mmol) in dichloromethane (50 mL) at 0° C. was added 4N HCl in dioxane (6.8 mL). The reaction mixture was stirred at room temperature overnight. The precipitate was collected by filtration to afford the product as a colorless solid, 4.4 g (86%).

MS (ESP): 343 (MH⁺) for C₂₀H₂₃FN₂O₂

### Intermediate 27

### Cis(±)tert-butyl 4-{benzyl[(benzyloxy)carbonyl]amino}-3-fluoropineridine-1-carboxylate

To a mixture of cis(±) tert-butyl 4-(benzylamino)-3-fluoropiperidine-1-carboxylate (Intermediate 28, 1.1 g, 3.6 mmol) in dioxane (20 mL) and saturated sodium carbonate (10 mL) at 0° C. was added dropwise benzyl chloroformate (0.76 mL, 5.4 mmol) and the reaction mixture was stirred at 0° C. for 1 hour. Ethyl acetate (˜20 mL) and brine (˜20 mL) were added and the layers were separated. The aqueous phase was extracted once with ethyl acetate and the combined organic phases were dried over magnesium sulfate and concentrated under reduced pressure to give the product as a colorless solid, 1.4 g (89%).

MS (ESP): 343 (−BOC, MH⁺) for C₂₅H₃₁FN₂O₄

¹H-NMR (CDCl₃-d) δ: 1.46 (s, 9H); 1.46 (m, 1H); 2.00 (m, 1H); 2.91 (m, 2H); 4.33 (m, 4H); 4.86 (m, 2H); 5.16 (m, 2H); 7.28 (m, 10H).

### Intermediate 28

### Cis(±)tert-butyl 4-(benzylamino)-3-fluoropiperidine-1-carboxylate

A mixture of tert-butyl 3-fluoro-4-oxopiperidine-1-carboxylate (Intermediate 29, 8.1 g, 37.3 mmol), benzylamine (4.5 mL, 41 mmol) and 3 Å molecular sieves in dichloromethane (150 mL) was treated at 0° C. with sodium triacetoxyborohydride (11.8 g, 56 mmol) in portions. The reaction mixture was stirred at room temperature for 30 minutes and then filtered. Saturated aqueous sodium bicarbonate was added and the layers were separated. The aqueous layer was extracted once with dichloromethane. The combined organic phases were dried over magnesium sulfate and concentrated under reduced pressure. Silica gel chromatography with hexanes/ethyl acetate (3:2) afforded the product as an off-white solid, 6.9 g (60%).

MS (ESP): 309 (MH⁺) for C₁₇H₂₅FN₂O₂

### Intermediate 29

### tert-Butyl 3-fluoro-4-oxopiperidine-1-carboxylate

A solution of tert-butyl 4-[(trimethylsilyl)oxy]-3,6-dihydropyridine-1(2H)-carboxylate (Intermediate 30, 14 g, 51 mmol) in acetonitrile (200 mL) was treated at 0° C. with SELECTFLUOR™ (20 g, 57 mmol) in portions. The reaction mixture was stirred at 0° C. for 30 minutes, then diluted with ethyl acetate, washed with saturated sodium chloride, dried over magnesium sulfate and concentrated. Silica gel chromatography with hexanes/ethyl acetate (3:2) afforded the product as a colorless oil, 8.1 g (72%).

¹H-NMR (CDCl₃-d) δ: 1.48 (s, 9H); 2.56 (m, 2H); 3.22 (m, 2H); 4.18 (m, 1H); 4.45 (m, 1H); 4.72 (m, 1H).

### Intermediate 30

### tert-Butyl 4-[(trimethylsilyl)oxy]-3,6-dihydropyridine-1(2H)-carboxylate

A mixture of tert-butyl 4-oxo-1-piperidinecarboxylate (11 g, 55 mmol) and triethylamine (18.5 mL, 132 mmol) in DMF (40 mL) was treated dropwise at 0° C. with chlorotrimethylsilane (8.4 mL, 66 mmol). The reaction mixture was heated at 80° C. overnight, and then cooled to room temperature. Saturated sodium bicarbonate was added and the product was extracted twice with hexanes. The combined organic extracts were dried over magnesium sulfate and concentrated to give 14 g (93%) of product as a yellow oil.

¹H-NMR (CDCl₃) δ: 0.18 (s, 9H); 1.45 (s, 9H); 2.09 (m, 2H); 3.51 (m, 2H); 3.86 (m, 2H); 4.78 (m, 1H).

### Intermediate 31

### 1-{2-[(3S,4R)-4-Amino-3-methoxypiperidin-1-yl]ethyl}-7-methoxyquinoxalin-2(1H)-one

A solution of tert-butyl {(3S,4R)-3-methoxy-1-[2-(7-methoxy-2-oxoquinoxalin-1(2H)-yl)ethyl]piperidin-4-yl}carbamate (Intermediate 32, 420 mg, 0.97 mmol) in dichloromethane (50 mL) and treated with trifluoroacetic acid (10 mL). After 1 hour, the reaction was concentrated to dryness. The residue was taken up in 15% methanol in chloroform (30 mL) and washed with saturated sodium bicarbonate solution. The aqueous layer was re-extracted with 15% methanol/chloroform (4×30 mL). The combined organic phases were dried over magnesium sulfate and concentrated to dryness giving 310 mg (97%) of the crude product as an oil.

MS (ESP): 333 (MH⁺) for C₁₇H₂₄N₄O₃

### Intermediate 32

### tert-Butyl {(3S,4R)-3-methoxy-1-[2-(7-methoxy-2-oxoquinoxalin-1(2H)-yl)ethyl]piperidin-4-yl}carbamate

A solution of 7-methoxyquinoxalin-2(1H)-one (Intermediate 15, 320 mg, 1.79 mmol) in dry DMF (10 mL) was cooled in an ice bath under nitrogen and treated with sodium hydride (60% in oil, 86 mg, 2.15 mmol). The reaction was stirred at room temperature for ˜90 min. The reaction was again cooled in an ice bath and treated with a solution of 2-{(3S,4R)-4-[(tert-butoxycarbonyl)amino]-3-methoxypiperidin-1-yl}ethyl methanesulfonate in dry DMF (Intermediate 33, ˜0.20 mmol/mL, 1.97 mmol). The reaction was stirred at room temperature overnight, then concentrated to dryness under reduced pressure. The residue was partitioned between ethyl acetate and water. The aqueous phase was re-extracted 2× with ethyl acetate. The combined organic layers were dried over magnesium sulfate. Chromatography on silica gel with a gradient of 15-25% acetone in hexanes gave 420 mg (55%) of the product as a colorless solid.

MS (ESP): 433 (MH⁺) for C₂₂H₃₂N₄O₅

¹H NMR (DMSO-d₆) δ: 1.38 (s, 9H); 1.43-1.51 (m, 1H); 1.57-1.72 (m, 1H); 2.20-2.40 (m, 2H); 2.55-2.66 (m, 2H); 2.67-2.78 (m, 1H); 2.80-2.93 (m, 1H); 3.18 (s, 3H); 3.29 (s, 1H); 3.51-3.65 (m, 1H); 3.92 (s, 3H); 4.24-4.43 (m, 2H); 6.40 (d, 1H); 6.96-7.05 (m, 2H); 7.75 (d, 1H); 8.04 (s, 1H).

### Intermediate 33

### 2-{(3S,4R)-4-[(tert-Butoxycarbonyl)amino]-3-methoxypiperidin-1-yl}ethyl methanesulfonate

A solution of tert-butyl [(3S,4R)-1-(2-hydroxyethyl)-3-methoxypiperidin-4-yl]carbamate (Intermediate 34, 540 mg, 1.97 mmol) in dry dichloromethane (20 mL) at 0° C. was treated with triethylamine (0.38 mL, 2.76 mmol) followed by methanesulfonyl chloride (0.18 mL, 2.36 mmol). After 15 min, TLC revealed complete disappearance of starting material. The reaction was quenched with potassium phosphate buffer (1 M, pH 7). The aqueous phase was re-extracted (1×) with dichloromethane. Ethyl acetate was added to the combined organic phases. Dichloromethane was removed under reduced pressure leaving behind the product in solution in ethyl acetate. This organic phase was washed with water to remove any remaining salts. The aqueous phase was re-extracted (1×) with ethyl acetate. The combined organic phases were dried over sodium sulfate and filtered. Dry DMF (10 mL) was added to the filtrate. Ethyl acetate was removed under reduced pressure leaving behind the product in DMF, which was used directly in the next step without further purification.

### Intermediate 34

### tert-Butyl [(3S,4R)-1-(2-hydroxyethyl)-3-methoxypiperidin-4-yl]carbamate

A solution of 2-[(3S,4R)-4-(dibenzylamino)-3-methoxypiperidin-1-yl]ethanol (Intermediate 35, 940 mg, 2.66 mmol) and di-tert-butyl dicarbonate (0.67 mL, 2.92 mmol) in methanol (100 mL) was hydrogenated over 20% palladium hydroxide on carbon (240 mg) overnight. The reaction mixture was filtered through celite and concentrated to dryness under reduced pressure. Chromatography on silica gel with 2-10% methanol in chloroform gave 540 mg (74%) of the product as a colorless oil.

¹H NMR (DMSO-d₆) δ: 1.38 (s, 9H); 1.43-1.50 (m, 1H); 1.58-1.73 (m, 1H); 2.15 (d, 2H); 2.37 (t, 2H); 2.54-2.64 (m, 1H); 2.75-2.88 (m, 1H); 3.22 (s, 3H); 3.27-3.32 (m, 1H); 3.41-3.59 (m, 3H); 4.37 (t, 1H); 6.35 (d, 1H).

### Intermediate 35

### 2-[(3S,4R)-4-(Dibenzylamino)-3-methoxypiperidin-1-yl]ethanol

and

### Intermediate 36

### 2-[(3R,4S)-4-(Dibenzylamino)-3-methoxypiperidin-1-yl]ethanol

A mixture of cis(±)N,N-dibenzyl-3-methoxypiperidin-4-amine (1.7 g, 5.5 mmol) (WO 2005/068461), bromoethanol (0.5 mL, 7.1 mmol), and N,N-diisopropylethylamine (1.4 mL, 8.3 mmol) were reacted like described for Intermediate 7, but heating for one hour at 70° C. Chromatography on silica gel with 5% methanol in dichloromethane containing 0.25% ammonium hydroxide gave 1.3 g (68%) of the cis-racemic product as a colorless solid.

MS (ESP): 355 (MH⁺) for C₂₂H₃₀N₂O₂

¹H NMR (DMSO-d₆) δ: 1.44-1.58 (m, 1H); 1.64 (d, 1H); 1.79-2.08 (m, 2H); 2.32 (t, 2); 2.36-2.45 (m, 1H); 2.88 (d, 1H); 3.13 (d, 1H); 3.30 (s, 3H); 3.40-3.49 (m, 2H); 3.56 (s, 1H); 3.59-3.87 (m, 4H); 4.34 (s, 1H); 7.11-7.24 (m, 2H); 7.24-7.40 (m, 8H).

The enantiomers were separated by chiral chromatography on a chiral cell OJ column (250×20 mm, 10 micron) eluting with 1:1 methanol/ethanol and 0.1% diethylamine at 10 mL/min flow rate. The (−) isomer (Intermediate 36) eluted first followed by the (+) isomer (Intermediate 35).

### Intermediate 37

### 1-{2-[(3R,4S)-4-Amino-3-methoxypiperidin-1-yl]ethyl}-7-methoxyquinoxalin-2(1H)-one

tert-Butyl {(3R,4S)-3-methoxy-1-[2-(7-methoxy-2-oxoquinoxalin-1(2H)-yl)ethyl]piperidin-4-yl}carbamate (Intermediate 38, 100 mg, 0.23 mmol) was reacted with trifluoroacetic acid using a procedure similar to the one described for the synthesis of Intermediate 31 to give 70 mg (91%) of the crude product as an oil.

MS (ESP): 333 (MR) for C₁₇H₂₄N₄O₃

### Intermediate 38

### tert-Butyl {(3R,4S)-3-methoxy-1-[2-(7-methoxy-2-oxoquinoxalin-1(2H)-yl)ethyl]piperidin-4-yl}carbamate

7-Methoxyquinoxalin-2(1H)-one (Intermediate 15, 530 mg, 3.00 mmol), 2-{(3R,4S)-4-[(tert-butoxycarbonyl)amino]-3-methoxypiperidin-1-yl}ethyl methanesulfonate (Intermediate 39, ˜0.33 mmol/mL, 3.30 mmol), and sodium hydride (60% in oil, 140 mg, 3.60 mmol) were reacted using a procedure similar to the one described for the synthesis of Intermediate 32. The crude product was purified by flash chromatography eluting with 25% acetone in hexanes to give 540 mg (42%) of the product as an off white solid.

MS (ESP): 433 (MH⁺) for C₂₂H₃₂N₄O₅

¹H NMR (DMSO-d₆) δ: 1.38 (s, 9H); 1.42-1.50 (m, 1H); 1.57-1.72 (m, 1H); 2.20-2.38 (m, 2H); 2.60 (t, 2H); 2.68-2.78 (m, 1H); 2.79-2.93 (m, 1H); 3.18 (s, 3H); 3.25-3.31 (m, 1H); 3.50-3.67 (m, 1H); 3.92 (s, 3H); 4.23-4.42 (m, 2H); 6.41 (d, 1H); 6.96-7.07 (m, 2H); 7.75 (d, 1H); 8.04 (s, 1H).

### Intermediate 39

### 2-{(3R,4S)-4-[(tert-Butoxycarbonyl)amino]-3-methoxypiperidin-1-yl}ethyl methanesulfonate

tert-Butyl [(3R,4S)-1-(2-hydroxyethyl)-3-methoxypiperidin-4-yl]carbamate (Intermediate 40, 0.91 g, 3 3 mmol), triethylamine (0.64 mL, 4.62 mmol), and methanesulfonyl chloride (0.31 mL, 3.99 mmol) were reacted as described for Intermediate 33. The crude product was used directly in the next step without further purification.

### Intermediate 40

### tert-Butyl [(3R,4S)-1-(2-hydroxyethyl)-3-methoxypiperidin-4-yl]carbamate

2-[(3R,4S)-4-(Dibenzylamino)-3-methoxypiperidin-1-yl]ethanol (Intermediate 36, 3.2 g, 9.0 mmol) was reacted using a procedure similar to the one described for the synthesis of Intermediate 34 to give 1.7 g (68%) of the product as a colorless oil.

¹H-NMR (DMSO-d₆) δ: 1.38 (s, 9H); 1.42-1.50 (m, 1H); 1.58-1.73 (m, 1H); 2.11-2.21 (m, 2H); 2.38 (t, 2H); 2.55-2.66 (m, 1H); 2.77-2.89 (m, 1H); 3.23 (s, 3H); 3.28-3.33 (m, 1H); 3.41-3.59 (m, 3H); 4.38 (s, 1H); 6.36 (d, 1H).

### Intermediate 41

### 1-{2-[(3S,4R)-4-Amino-3-methoxypiperidin-1-yl]ethyl}-2-oxo-1,2-dihydroquinoline-7-carbonitrile

tert-Butyl {(3S,4R)-1-[2-(7-cyano-2-oxoquinolin-1(2H)-yl)ethyl]-3-methoxypiperidin-4-yl}carbamate (Intermediate 42, 370 mg, 0.87 mmol) was reacted with trifluoroacetic acid in dichloromethane using a procedure similar to the one described for the synthesis of Intermediate 31 to give 300 mg (quant.) of the crude product as an oil.

MS (ESP): 327 (MH⁺) for C₁₈H₂₂N₄O₂

### Intermediate 42

### tert-Butyl {(3S,4R)-1-[2-(7-Cyano-2-oxoquinolin-1(2H)-yl)ethyl]-3-methoxypiperidin-4-yl}carbamate

2-Oxo-1,2-dihydroquinoline-7-carbonitrile (Intermediate 5, 370 mg, 2.20 mmol), 2-{(3S,4R)-4-[(tert-butoxycarbonyl)amino]-3-methoxypiperidin-1-yl}ethyl methanesulfonate (Intermediate 33, ˜0.24 mmol/mL, 2.40 mmol), and sodium hydride (60% in oil, 110 mg, 2.60 mmol) were reacted using a procedure similar to the one described for the synthesis of Intermediate 32. Chromatography on silica gel with 25-35% acetone in hexanes giving 370 mg (39%) of the product as an off white solid.

MS (ESP): 427 (MH⁺) for C₂₃H₃₀N₄O₄

¹H-NMR (DMSO-d₆) δ: 1.38 (s, 9H); 1.42-1.51 (m, 1H); 1.57-1.71 (m, 1H); 2.18-2.40 (m, 2H); 2.56 (t, 2H); 2.65-2.76 (m, 1H); 2.78-2.90 (m, 1H); 3.18 (s, 3H); 3.27 (s, 1H); 3.58 (s, 1H); 4.30-4.46 (m, 2H); 6.37 (d, 1H); 6.78 (d, 1H); 7.66 (dd, 1H); 7.91 (d, 1H); 8.01 (d, 1H); 8.09 (s, 1H).

### Intermediate 43

### 1-{2-[(3R,4S)-4-Amino-3-methoxypiperidin-1-yl]ethyl}-2-oxo-1,2-dihydroquinoline-7-carbonitrile

tert-Butyl {(3R,4S)-1-[2-(7-cyano-2-oxoquinolin-1(2H)-yl)ethyl]-3-methoxypiperidin-4-yl}carbamate (Intermediate 44, 320 mg, 0.75 mmol) was reacted with trifluoroacetic acid using a procedure similar to the one described for the synthesis of Intermediate 31 to give 250 mg (quant.) of the crude product as an oil.

MS (ESP): 327 (MH⁺) for C₁₈H₂₂N₄O₂

### Intermediate 44

### tert-Butyl {(3R,4S)-1-[2-(7-cyano-2-oxoquinolin-1(2H)-yl)ethyl]-3-methoxypiperidin-4-yl}carbamate

A mixture of tert-butyl {(3R,4S)-1-[2-(7-bromo-2-oxoquinolin-1(2H)-yl)ethyl]-3-methoxypiperidin-4-yl}carbamate (Intermediate 45, 460 mg, 0.98 mmol) and potassium cyanide (96 mg, 1.5 mmol) in acetonitrile (10 mL) was degassed and purged with nitrogen (3 times). A solution of tributyl tin chloride (14 μL/mL in heptane, 0.90 μL, 0.003 mmol), 4,5-bis(diphenylphosphino)-9,9-dimethylxanthene (3 mg, 0.005 mmol), and tris(dibenzylideneacetone)dipalladium (O) (5 mg, 0.005 mmol) was added. The mixture was degassed 3× and stirred at room temperature for 30 min. The mixture was degassed once more then heated to 80° C. overnight. LC/MS revealed incomplete conversion to product. More tributyl tin chloride (14 μL/mL in heptane, 0.90 μL, 0.003 mmol), 4,5-bis(diphenylphosphino)-9,9-dimethylxanthene (3 mg, 0.005 mmol), and tris(dibenzylideneacetone)dipalladium (0) (5 mg, 0.005 mmol) was added. The reaction was stirred again overnight resulting in complete conversion to product. The reaction was diluted with dichloromethane. The organic phase was washed with water. The aqueous phase was re-extracted 4× with dichloromethane. The combined organic phases were dried over sodium sulfate and concentrated to. Chromatography on silica gel with 25-35% acetone in hexanes gave 320 mg (76%) of the product as a yellow solid.

MS (ESP): 427 (MH⁺) for C₂₃H₃₀N₄O₄

¹H NMR (DMSO-d₆) δ: 1.33-1.41 (m, 9H); 1.42-1.50 (m, 1H); 1.56-1.73 (m, 1H); 2.17-2.41 (m, 2H); 2.57 (t, 2H); 2.64-2.76 (m, 1H); 2.78-2.91 (m, 1H); 3.13-3.22 (m, 3H); 3.25-3.30 (m, 1H); 3.52-3.66 (m, 1H); 4.29-4.44 (m, 2H); 6.38 (d, 1H); 6.79 (d, 1H); 7.65 (dd, 1H); 7.92 (d, 1H); 8.01 (d, 1H); 8.09 (d, 1H).

### Intermediate 45

### tert-Butyl {(3R,4S)-1-[2-(7-bromo-2-oxoquinolin-1(2H)-yl)ethyl]-3-methoxypiperidin-4-yl}carbamate

7-Bromoquinolin-2(1H)-one (Intermediate 46, 450 mg, 2.00 mmol), 2-{(3R,4S)-4-[(tert-butoxycarbonyl)amino]-3-methoxypiperidin-1-yl}ethyl methanesulfonate (Intermediate 39, ˜0.23 mmol/mL, 2.30 mmol), and sodium hydride (60% in oil, 100 mg, 2.61 mmol) were reacted using a procedure similar to the one described for the synthesis of Intermediate 32. The crude product was purified by flash chromatography eluting with a gradient of 15-30% acetone in hexanes giving 460 mg (48%) of the product as an off white solid.

MS (ESP): 480/482 (MH⁺) for C₂₂H₃₀BrN₃O₄

¹H NMR (DMSO-d₆) δ: 1.36-1.41 (m, 9H); 1.43-1.52 (m, 1H); 1.59-1.75 (m, 1H); 2.20-2.38 (m, 2H); 2.54 (t, 2H); 2.63-2.75 (m, 1H); 2.77-2.89 (m, 1H); 3.16-3.23 (m, 3H); 3.26-3.32 (m, 1H); 3.52-3.68 (m, 1H); 4.33 (t, 2H); 6.40 (d, 1H); 6.64 (d, 1H); 7.44 (dd, 1H); 7.68 (d, 1H); 7.73-7.78 (m, 1H); 7.91 (d, 1H).

### Intermediate 46

### 7-Bromoquinolin-2(1H)-one

(2E)-N-(3-bromophenyl)-3-phenylacrylamide (Intermediate 47, 16 g, 53 mmol) and aluminium trichloride (31.8 g, 238 mmol) were heated in chlorobenzene (100 mL) at 90° C. bath temperature for one hour. The reaction mixture was cooled to room temperature and poured onto ice. It was stirred until the ice had completely molten, the mixture was filtered and washed with water and ethyl acetate to give the crude product as slightly brown solid in a mixture with the minor product 5-bromoquinolin-2(1H)-one (˜3:2), 8.8 g (70%). This mixture could not be separated. The mixture was heated in phosphoroxychloride (50 mL) at 65° C. for one hour. The reaction mixture was cooled to room temperature and poured onto ice. It was carefully neutralized at 0° C. with sodium carbonate, extracted into ethyl acetate (300 mL), washed with brine, dried over sodium sulfate and concentrated to give the crude mixture of 7-bromo-2-chloroquinoline and 5-bromo-2-chloroquinoline. The mixture was taken up in dichloromethane (100 mL), treated with silica gel (˜20 g), filtered and the filter cake was washed with dichloromethane. Filtrate and wash were combined and concentrated. The residue was crystallized from toluene/hexanes (˜70 mL, 1:1) to provide pure 7-bromo-2-chloroquinoline, 3.74 g as a colorless solid mp 113° C.

¹H-NMR (DMSO-d₆) δ: 7.63 (d, J 8.4 Hz, 1H); 7.81 (dd, J 8.4, 1.6 Hz, 1H); 8.03 (d, J 8.4 Hz, 1H); 8.18 (d, J 1.6 Hz, 1H); 8.48 (d, J 8.4 Hz, 1H).

MS (ESP): 242/244/246 (MH⁺) for C₉H₅BrClN

This chloride was heated in 5M HCl (100 mL) and dioxane (10 mL) for 1 hour at reflux. It was cooled, filtered and washed with water to give the title compound, 2.89 g, as a colorless solid, mp 295° C.

MS (ESP): 224.13/226.13 (MH⁺) for C₉H₆BrNO

¹H-NMR (DMSO-d₆) δ: 6.51 (d, J 9.6 Hz, 1H); 7.32 (dd, J 8.6, 1.6 Hz, 1H); 7.46 (d, J 1.6 Hz, 1H); 7.61 (d, J 8.6 Hz, 1H); 7.88 (d, J 9.6 Hz, 1H); 11.80 (brs, 1H).

### Intermediate 47

### (2E)-N-(3-Bromophenyl)-3-phenylacrylamide

To a solution of 3-bromoaniline (13.1 mL, 120 mmol) in dichloromethane (100 mL) and 2,6-lutidine (21 mL, 180 mmol) at 0° C. was added a solution of cinnamoylchloride (20 g, 120 mmol) in dichloromethane (50 mL) dropwise. The reaction mixture was allowed to reach room temperature and stirred for 2 hours. It was quenched with potassium phosphate buffer (100 mL, 1M, pH 7) and stirred for 15 minutes. Dichloromethane was removed under reduced pressure and it was extracted with ethyl acetate. The organic phase was washed with phosphate buffer (like above, 200 mL), dried over sodium sulfate and concentrated to dryness. The residue was crystallized from toluene/hexanes to give the product as colorless solid (33.4 g, 92%).

MS (ESP): 302/304 (MH⁺) for C₁₅H₁₂BrNO

¹H-NMR (DMSO-d₆) δ: 6.79 (d, 1H); 7.23-7.70 (m, 9H); 8.07 (s, 1H); 10.38 (s, 1H).

### Intermediate 48

### 1-{2-[(3S,4S)-4-Amino-3-methoxypiperidin-1-yl]ethyl}-7-methoxyquinoxalin-2(1H)-one

tert-Butyl {(3S,4S)-3-methoxy-1-[2-(7-methoxy-2-oxoquinoxalin-1(2H)-yl)ethyl]piperidin-4-yl}carbamate, (−) trans enantiomer (Intermediate 49, 190 mg, 0.44 mmol) was reacted with trifluoroacetic as described for Intermediate 31 to give 140 mg (93%) of the crude product as an oil.

MS (ESP): 333 (MH⁺) for C₁₇H₂₄N₄O₃

### Intermediate 49

### tert-Butyl {(3S, 4S)-3-methoxy-1-[2-(7-methoxy-2-oxoquinoxalin-1(2H)-yl)ethyl]piperidin-4-yl}carbamate, (−) trans enantiomer

and

### Intermediate 50

### tert-Butyl {(3R,4R)-3-methoxy-1-[2-(7-methoxy-2-oxoquinoxalin-1(2H)-yl)ethyl]piperidin-4-yl}carbamate, (+) trans enantiomer

7-Methoxyquinoxalin-2(1H)-one (Intermediate 15, 430 mg, 2.45 mmol), 2-{trans(±)-4-[(tert-butoxycarbonyl)amino]-3-methoxypiperidin-1-yl}ethyl methanesulfonate (Intermediate 51, ˜0.27 mmol/mL, 2.70 mmol), and sodium hydride (60% in oil, 110 mg, 2.70 mmol) were reacted using a procedure similar to the one described for the synthesis of Intermediate 32. The crude product was purified by flash chromatography eluting with a gradient of 15-35% acetone/hexanes to give 490 mg (45%) of the racemic mixture of the products as an off white solid.

MS (ESP): 433 (MH⁺) for C₂₂H₃₂N₄O₅

¹H NMR (DMSO-d₆) δ: 1.21-1.33 (m, 1H); 1.37 (s, 9H); 1.63-1.74 (m, 1H); 1.78 (t, 1H); 2.01 (t, 1H); 2.62 (t, 2H); 2.80-2.90 (m, 1H); 2.96-3.06 (m, 1H); 3.07-3.18 (m, 1H); 3.22-3.29 (m, 4H); 3.93 (s, 3H); 4.27-4.43 (m, 2H); 6.78 (d, 1H); 6.96-7.06 (m, 2H); 7.75 (d, 1H); 8.05 (s, 1H).

The mixture of enantiomers was separated by supercritical fluid chromatography on a Chiralpak AD-H column (250×21 mm, 5 micron) eluting with an isocratic gradient of 20% isopropanol/0.1% dimethylethylamine at a flow rate of 60 mL/min. This gave 190 mg of tert-butyl {(3S,4S)-3-methoxy-1-[2-(7-methoxy-2-oxoquinoxalin-1(2H)-yl)ethyl]piperidin-4-yl}carbamate (Intermediate 49) (first eluting compound, (−) trans enantiomer) and 190 mg of tert-butyl {(3R,4R)-3-methoxy-1-[2-(7-methoxy-2-oxoquinoxalin-1(2H)-yl)ethyl]piperidin-4-yl}carbamate (Intermediate 50) (second eluting compound, (+) trans enantiomer).

### Intermediate 51

### 2-{trans(±)-4-[(tert-Butoxycarbonyl)amino]-3-methoxypiperidin-1-yl}ethyl methanesulfonate

tert-Butyl [trans(±)-1-(2-hydroxyethyl)-3-methoxypiperidin-4-yl]carbamate (Intermediate 52, 0.74 g, 2.7 mmol), triethylamine (0.53 mL, 3.78 mmol), and methanesulfonyl chloride (0.25 mL, 3.24 mmol) were reacted using a procedure similar to the one described for the synthesis of Intermediate 33. The crude product was used directly in the next step without further purification.

### Intermediate 52

### tert-Butyl [trans(±)-1-(2-hydroxyethyl)-3-methoxypiperidin-4-yl]carbamate

tert-Butyl [trans(±)-3-methoxypiperidin-4-yl]carbamate (Intermediate 53, 1.1 g, 4.8 mmol), 2-bromoethanol (0.44 mL, 6.2 mmol), and ethyl(diisopropyl)amine (1.25 mL, 7.2 mmol) were reacted using a procedure similar to the one described for the synthesis of Intermediate 7 to give 0.74 g (57%) of the product as a colorless oil.

¹H-NMR (DMSO-d₆) δ: 1.24-1.34 (m, 1H); 1.38 (s, 9H); 1.62-1.77 (m, 2H); 1.82-1.97 (m, 1H); 2.38 (t, 2H); 2.73 (d, 1H); 2.98-3.18 (m, 3H); 3.27 (s, 3H); 3.46 (q, 2H); 4.39 (t, 1H); 6.78 (d,

### Intermediate 53

### tert-Butyl [trans(±)-3-methoxypiperidin-4-yl]carbamate

Benzyl trans(±)-4-[(tert-butoxycarbonyl)amino]-3-methoxypiperidine-1-carboxylate (Intermediate 54, 0.98 g, 2.69 mmol) was hydrogenated in methanol (50 mL) over 10% Pd/C (400 mg) at normal pressure. After 1 hour the reaction mixture was filtered through celite. The filtrate was concentrated to dryness giving 0.61 g (98%) of the product as a colorless oil.

¹H NMR (DMSO-d₆) δ: 1.14-1.29 (m, 1H); 1.34-1.42 (m, 9H); 1.68 (d, 1H); 2.11 (dd, 1H); 2.26-2.38 (m, 1H); 2.71-2.82 (m, 1H); 2.86-2.98 (m, 1H); 3.14-3.21 (m, 3H); 3.26 (s, 3H); 6.75-6.86 (m, 1H).

### Intermediate 54

### Benzyl trans(±)-4-[(tert-butoxycarbonyl)amino]-3-methoxypiperidine-1-carboxylate

Benzyl trans(±)-4-[(tert-butoxycarbonyl)amino]-3-hydroxypiperidine-1-carboxylate (Intermediate 55, 1.0 g, 2.86 mmol) was suspended in 10 mL of toluene and treated with a 50 wt % solution of aqueous sodium hydroxide (6 mL) followed by dimethylsulfate (0.33 mL, 3.43 mmol) and benzyl triethylammonium chloride (catalytic amount). The reaction was stirred vigorously for one hour. The reaction was quenched with ice. The phases were separated. The aqueous phase was re-extracted with ethyl acetate. The combined organic phases were dried over sodium sulfate and concentrated to dryness. Chromatography on silica gel with 25-50% acetone in hexanes gave 0.78 g (78%) of the product as a colorless oil.

MS (ESP): 365 (MH⁺) for C₁₉H₂₈N₂O₅

¹H NMR (DMSO-d₆) δ: 0.55-0.68 (m, 10H); 1.04-1.19 (m, 1H); 2.17-2.46 (m, 2H); 2.55-2.64 (m, 2H); 2.67-2.80 (m, 1H); 2.85-3.07 (m, 1H); 3.10-3.31 (m, 1H); 4.09 (s, 3H); 4.32 (s, 2H); 6.45-6.61 (m, 5H).

### Intermediate 55

### Benzyl trans(±)-4-[(tert-butoxycarbonyl)amino]-3-hydroxypiperidine-1-carboxylate

A mixture of benzyl trans(±)-4-amino-3-hydroxypiperidine-1-carboxylate (WO 2005/066176, 3.0 g, 12.0 mmol), di-tert-butyl dicarbonate (2.9 g, 13.2 mmol) and sodium bicarbonate (3.0 g, 36.0 mmol) in ethyl acetate/water (1:1, 100 mL) was stirred vigorously overnight. The biphasic mixture was separated. The aqueous phase was re-extracted 1× with ethyl acetate. The combined organic phases were dried over sodium sulfate and concentrated to dryness giving 4.2 g of the product as a colorless solid. This material was used without further purification.

¹H NMR (DMSO-d₆) δ: 1.15-1.32 (m, 1H); 1.35-1.42 (m, 9H); 1.71-1.83 (m, 1H); 2.60-2.79 (m, 1H); 2.82-2.98 (m, 1H); 3.15-3.29 (m, 2H); 3.74-3.86 (m, 1H); 3.88-3.98 (m, 1H); 5.00 (d, 1H); 5.04-5.08 (m, 2H); 6.73 (d, 1H); 7.25-7.42 (m, 5H).

### Intermediate 56

### 1-{2-[(3R,4R)-4-Amino-3-methoxypiperidin-1-yl]ethyl}-7-methoxyquinoxalin-2(1H)-one

tert-Butyl {(3R,4R)-3-methoxy-1-[2-(7-methoxy-2-oxoquinoxalin-1(2H)-yl)ethyl]piperidin-4-yl}carbamate, (+) trans enantiomer (Intermediate 50, 190 mg, 0.44 mmol) was reacted with trifluoroacetic acid using a procedure similar to the one described for the synthesis of Intermediate 31 to give 150 mg (quant.) of the crude product as an oil.

MS (ESP): 333 (MH⁺) for C₁₇H₂₄N₄O₃

### Intermediate 57

### 1-{2-[(4-Amino-3-hydroxypiperidin-1-yl]ethyl}-7-methoxyquinoxalin-2(1H)-one, trans enantiomer A

tert-Butyl {(3-hydroxy-1-[2-(7-methoxy-2-oxoquinoxalin-1(2H)-yl)ethyl]piperidin-4-yl}carbamate, trans enantiomer A (Intermediate 58, 130 mg, 0.31 mmol) was reacted with trifluoroacetic acid as described for Intermediate 31 to give 78 mg (79%) of the crude product as an oil.

MS (ESP): 319 (MH⁺) for C₁₆H₂₂N₄O₃

### Intermediate 58

### tert-Butyl {3-hydroxy-1-[2-(7-methoxy-2-oxoquinoxalin-1(2H)-yl)ethyl]piperidin-4-yl}carbamate, trans enantiomer A

and

### Intermediate 59

### tert-Butyl {3-hydroxy-1-[2-(7-methoxy-2-oxoquinoxalin-1(2H)-yl)ethyl]piperidin-4-yl}carbamate, trans enantiomer B

A solution of tert-butyl {trans(±)-3-{[tert-butyl(dimethyl)silyl]oxy}-1-[2-(7-methoxy-2-oxoquinoxalin-1(2H)-yl)ethyl]piperidin-4-yl}carbamate (Intermediate 60, 0.60 g, 1.13 mmol) in THF (20 mL) was treated at 0° C. with a solution of tetrabutylammonium fluoride in THF (1M, 2.2 mL). The reaction was stirred at room temperature for 2 hours, then concentrated to dryness under reduced pressure. The crude residue was taken up in ethyl acetate. The organic phase was washed with water. The aqueous phase was re-extracted 3 times with ethyl acetate. The combined organic phases were dried over sodium sulfate and concentrated to. Chromatography on silica gel with 0-5% methanol in dichloromethane gave 0.27 g of the desired product and 0.12 g of an O-acetylated side product. The side product was taken up in methanol and treated with a catalytic amount of potassium carbonate. This was stirred at room temperature for one hour resulting in complete conversion to the alcohol. The reaction mixture was concentrated to dryness. The residue was partitioned between aqueous potassium phosphate buffer (pH=7) and ethyl acetate. The aqueous phase was re-extracted 2× with ethyl acetate. The combined organic phases were dried over sodium sulfate, filtered, and concentrated to dryness giving an additional 100 mg of the desired (79% total) of product.

MS (ESP): 419 (MH⁺) for C₂₁H₃₀N₄O₅

¹H NMR (DMSO-d₆) δ: 1.18-1.33 (m, 1H); 1.38 (s, 9H); 1.63-1.77 (m, 1H); 1.86 (t, 1H); 1.97 (t, 1H); 2.54-2.64 (m, 2H); 2.80-2.93 (m, 1H); 2.96-3.09 (m, 2H); 3.23 (dd, 1H); 3.92 (s, 3H); 4.32 (t, 2H); 4.67 (d, 1H); 6.62 (d, 1H); 6.94-7.06 (m, 2H); 7.69-7.79 (m, 1H); 8.04 (s, 1H).

The mixture of enantiomers was separated by supercritical fluid chromatography on a Chiralpak AD-H column (250×21 mm, 5 micron) eluting with an isocratic gradient of 25% isopropanol/0.1% dimethylethylamine at a flow rate of 60 mL/min. This gave 130 mg of tert-butyl {3-hydroxy-1-[2-(7-methoxy-2-oxoquinoxalin-1(2H)-yl)ethyl]piperidin-4-yl}carbamate, trans enantiomer A (Intermediate 58, first eluting enantiomer) and 130 mg of tert-butyl {(3-hydroxy-1-[2-(7-methoxy-2-oxoquinoxalin-1(2H)-yl)ethyl]piperidin-4-yl}carbamate, trans enantiomer B (Intermediate 59, second eluting enantiomer).

### Intermediate 60

### tert-Butyl {trans(±)-3-{[tert-butyl(dimethyl)silyl]oxy}-1-[2-(7-methoxy-2-oxoquinoxalin-1(2H)-yl)ethyl]piperidin-4-yl}carbamate

7-Methoxyquinoxalin-2(1H)-one (Intermediate 15, 430 mg, 2.43 mmol), 2-(trans(±)-4-[(tert-butoxycarbonyl)amino]-3-{[tert-butyl(dimethyl)silyl]oxy}piperidin-1-yl)ethyl methanesulfonate (Intermediate 61, ˜0.27 mmol/mL, 2.70 mmol), and sodium hydride (60% in oil, 110 mg, 2.70 mmol) were reacted using a procedure similar to the one described for the synthesis of Intermediate 32. The crude product was purified by flash chromatography eluting with a gradient of 10-25% acetone/hexanes giving 600 mg (46%) of the product as an off white solid.

MS (ESP): 533 (MH⁺) for C₂₇H₄₄N₄O₅Si

¹H NMR (DMSO-d₆) δ: 0.00 (s, 6H); 0.79 (s, 9H); 1.33 (s, 9H); 1.37-1.47 (m, 1H); 1.49-1.59 (m, 1H); 1.87 (t, 1H); 1.95-2.07 (m, 1H); 2.55-2.66 (m, 2H); 2.77-2.89 (m, 1H); 2.92-3.02 (m, 1H); 3.11 (s, 1H); 3.30-3.40 (m, 1H); 3.89 (s, 3H); 4.17-4.41 (m, 2H); 6.57 (d, 1H); 6.91-7.04 (m, 2H); 7.72 (d, 1H); 8.01 (s, 1H).

### Intermediate 61

### 2-(trans(±)-4-[(tert-Butoxycarbonyl)amino]-3-{[tert-butyl(dimethyl)silyl]oxy}piperidin-1-yl)ethyl methanesulfonate

tert-Butyl [trans(±)-3-{[tert-butyl(dimethyl)silyl]oxy}-1-(2-hydroxyethyl)piperidin-4-yl]carbamate (Intermediate 62, 1.0 g, 2.7 mmol), triethylamine (0.52 mL, 3.74 mmol), and methanesulfonyl chloride (0.25 mL, 3.21 mmol) were reacted using a procedure similar to the one described for the synthesis of Intermediate 33. The crude product was used directly in the next step without further purification.

### Intermediate 62

### tert-Butyl [trans(±)-3-{[tert-butyl(dimethyl)silyl]oxy}-1-(2-hydroxyethyl)piperidin-4-yl]carbamate

tert-Butyl (trans(±)-3-{[tert-butyl(dimethyl)silyl]oxy}piperidin-4-yl)carbamate (Intermediate 63, 1.3 g, 3.9 mmol), 2-bromoethanol (0.36 mL, 5.1 mmol), and ethyl(diisopropyl)amine (1.0 mL, 5.9 mmol) were reacted using a procedure similar to the one described for the synthesis of Intermediate 7 to give 1.0 g (67%) of the desired product.

¹H NMR (DMSO-d₆) δ: 0.00 (s, 6H); 0.79 (s, 9H); 1.33 (s, 9H); 1.39 (dd, 1H); 1.46-1.58 (m, 1H); 1.71-1.82 (m, 1H); 1.82-1.93 (m, 1H); 2.33 (t, 2H); 2.72 (d, 1H); 2.80-2.90 (m, 1H); 2.99-3.16 (m, 1H); 3.32-3.47 (m, 3H); 4.36 (t, 1H); 6.56 (d, 1H).

### Intermediate 63

### tert-Butyl (trans(±)-3-{[tert-butyl(dimethyl)silyl]oxy}piperidin-4-yl)carbamate

Benzyl trans(±)-4-[(tert-butoxycarbonyl)amino]-3-{[tert-butyl(dimethyl)silyl]oxy}piperidine-1-carboxylate (Intermediate 64, 1.8 g, 3.9 mmol) was hydrogenated in methanol (50 mL) over 10% palladium on carbon (˜400 mg) at normal pressure for one hour. The reaction mixture was filtered through celite. The filtrate was concentrated under reduced pressure to give 1.3 g (quant.) of the product as a colorless solid.

¹H NMR (DMSO-d₆) δ: 0.00 (s, 6H); 0.80 (s, 9H); 1.20-1.30 (m, 1H); 1.33 (s, 9H); 1.53 (d, 1H); 2.15 (dd, 1H); 2.23-2.39 (m, 1H); 2.74 (d, 1H); 2.88 (dd, 1H); 3.20-3.30 (m, 2H); 4.08 (s, 1H); 6.58 (d, 1H).

### Intermediate 64

### Benzyl trans(±)-4-[(tert-butoxycarbonyl)amino]-3-{[tert-butyl(dimethyl)silyl]oxy}piperidine-1-carboxylate

A mixture of benzyl trans(±)-4-[(tert-butoxycarbonyl)amino]-3-hydroxypiperidine-1-carboxylate (Intermediate 55, 2.0 g, 5.7 mmol), imidazole (0.58 g, 8.6 mmol) and tert-butyl(chloro)dimethylsilane (1.0 g, 6.9 mmol) in DMF (15 mL) was stirred at room temperature under nitrogen overnight. Water (50 mL) was added to the reaction and the mixture was extracted 2× with ether. The combined organic phases were dried over sodium sulfate and concentrated to dryness. Chromatography on silica gel with 10-25% acetone in hexanes giving 1.8 g (69%) of the product as a colorless solid.

¹H NMR (DMSO-d₆) δ: 0.00 (s, 6H); 0.80 (s, 9H); 1.27-1.41 (m, 10H); 1.61-1.72 (m, 1H); 2.59-3.05 (m, 2H); 3.30-3.40 (m, 2H); 3.69-3.95 (m, 2H); 4.92-5.14 (m, 2H); 6.68 (d, 1H); 7.24-7.40 (m, 5H).

### Intermediate 65

### 1-{2-[4-Amino-3-hydroxypiperidin-1-yl]ethyl}-7-methoxyquinoxalin-2(1H)-one, trans enantiomer B

tert-Butyl {3-hydroxy-1-[2-(7-methoxy-2-oxoquinoxalin-1(2H)-yl)ethyl]piperidin-4-yl}carbamate trans enantiomer B (Intermediate 59, 130 mg, 0.31 mmol) was reacted with trifluoroacetic acid using a procedure similar to the one described for the synthesis of Intermediate 31 to give 84 mg (85%) of the crude product as an off white foam.

MS (ESP): 319 (MH⁺) for C₁₆H₂₂N₄O₃

### Intermediate 66

### 3-Hydroxy-2-oxo-1,2,3,4-tetrahydroquinoline-7-carbonitrile

Ethyl 3-(4-cyano-2-nitrophenyl)-2-oxopropanoate (6.5 kg, 24.8 mol) and acetonitrile (21 L) was stirred at 22° C., sodium borohydride (0.30 kg, 7.9 mol) was added in portions, the mixture was then stirred for 1 hour at 24° C. Acetic acid (65 L) was charged to the solution and the internal temperature was raised to 65° C. Iron (3.3 kg) was added to the solution in portions (6×0.5 kg) over 1 hour. After a further 1 hour the product was isolated by filtration, washed sequentially with water (3×25 L) and ethanol (29 L) and dried under reduced pressure to give the product as a beige solid, 3.07 kg (66%).

Mp>250° C.

MS (ESP): 189 (MH⁺) for C₁₀H₈N₂O₂

¹H-NMR (DMSO-d₆) δ ppm: 2.90-3.20 (m, 2H); 4.10-4.20 (m, 1H); 5.65 (d, 1H) 7.15 (s, 1H) 7.35-7.45 (m, 2H); 10.38 (s, 1H).

### Intermediate 67

### 1-{2-[-4-Amino-3-fluoropiperidin-1-yl]ethyl}-2-oxo-1,2-dihydroquinoline-7-carbonitrile, trans enantiomer A

To a solution of tert-butyl {1-[2-(7-cyano-2-oxoquinolin-1(2H)-yl)ethyl]-3-fluoropiperidin-4-yl}carbamate, trans enantiomer A (Intermediate 68, 0.30 g, 0.72 mmol) in dichloromethane (20 mL), was added trifluoroacetic acid (4 mL) with cooling in an ice bath. The reaction mixture was allowed to warm to room temperature. TLC revealed clean but incomplete conversion after ˜30 min (15% methanol/dichloromethane containing 0.5% ammonium hydroxide as eluent). Another 4 mL of trifluoroacetic acid was added. After 30 min the reaction was concentrated to dryness. The crude residue was partitioned between 15% methanol/dichloromethane and saturated sodium bicarbonate. The aqueous phase was adjusted to pH ˜10 with saturated sodium carbonate solution. The layers were separated. The aqueous phase was re-extracted 2× with 15% methanoUdichloromethane. The combined organic phases were dried over sodium sulfate, filtered, and concentrated to dryness giving 0.30 g of the crude product as an oil.

MS (ESP): 315 (MH⁺) for C₁₇H₁₉FN₄O₅

### Intermediate 68

### tert-Butyl {1-[2-(7-cyano-2-oxoquinolin-1(2H)-yl)ethyl]-3-fluoropiperidin-4-yl}carbamate, trans enantiomer A

A solution of 2-oxo-1,2-dihydroquinoline-7-carbonitrile (Intermediate 5, 500 mg, 2.94 mmol) in dry DMF (10 mL) was treated at 0° C. with sodium hydride (60% in oil, 153 mg, 3.82 mmol). Cooling was removed and the reaction was stirred at room temperature for 90 min. The reaction was again cooled in an ice bath and treated with a solution of 2-{(3R,4R)-4-[(tert-butoxycarbonyl)amino]-3-fluoropiperidin-1-yl}ethyl methanesulfonate, trans enantiomer A (Intermediate 69, ˜0.38 mmol/mL, 3.82 mmol), in dry DMF. The reaction was stirred at room temperature overnight. It was quenched with water (100 mL) and extracted with ethyl acetate (200 mL). The organic layer was washed with brine, dried over sodium sulfate, filtered, and concentrated in vacuo. Chromatography on silica gel with a gradient of 10-50% acetone in hexanes gave 680 mg (56%) of the product as an off white solid.

¹H NMR (DMSO-d₆) δ ppm: 1.25-1.43 (m, 11H); 1.67-1.78 (m, 1H); 2.04-2.17 (m, 2H); 2.57-2.68 (m, 2H); 2.80-2.89 (m, 1H); 3.25-3.32 (m, 1H); 4.27-4.47 (m, 2H); 4.30 (m, 1H) 6.78 (d, 1H); 6.99 (d, 1H); 7.66 (dd, 1H); 7.91 (d, 1H); 8.01 (d, 1H); 8.09 (s, 1H).

MS (ESP): 415 (MH⁺) for C₂₂H₂₇FN₄O₃

### Intermediate 69

### 2-{4-[(tert-Butoxycarbonyl)amino]-3-fluoropiperidin-1-yl}ethyl methanesulfonate, trans enantiomer A

A solution of tert-butyl [3-fluoro-1-(2-hydroxyethyppiperidin-4-yl]carbamate, trans enantiomer A (Intermediate 70, 2.0 g, 7.62 mmol) in dry dichloromethane (50 mL) at 0° C. was treated with triethylamine (1.5 mL, 10.7 mmol) followed by methanesulfonyl chloride (0.71 mL, 9.15 mmol). After 15 min, the reaction was quenched with potassium phosphate buffer (1M, pH 7). The aqueous phase was extracted once with dichloromethane. Ethyl acetate was added to the combined organic phases. Dichloromethane was removed under reduced pressure leaving behind the product in solution in ethyl acetate. This organic phase was washed with water to remove any remaining salts. The aqueous phase was re-extracted (1×) with ethyl acetate. The combined organic phases were dried over sodium sulfate and filtered. Dry DMF (20 mL) was added to the filtrate. Ethyl acetate was removed under reduced pressure leaving behind the product in DMF, which was used without delay in the next step without further purification.

### Intermediate 70

### tert-Butyl [3-fluoro-1-(2-hydroxyethyl)piperidin-4-yl]carbamate, trans enantiomer A

To a solution of benzyl benzyl[3-fluoro-1-(2-hydroxyethyl)piperidin-4-yl]carbamate, trans enantiomer A (Intermediate 71, 5.6 g, 14.5 mmol) in ethanol (100 mL) was added 20% palladium hydroxide on carbon (1.5 g). The reaction was stirred under an atmosphere of hydrogen overnight. TLC revealed complete disappearance of starting material (15% methanol/dichloromethane containing 0.5% ammonium hydroxide as eluent). The reaction mixture was then treated with di-tert-butyl dicarbonate (4.0 mL, 17.4 mmol) and stirred under nitrogen for 1 hour. TLC revealed complete disappearance of starting material. The reaction mixture was filtered through celite. The filtrate was concentrated to dryness and subjected to chromatography on silica gel eluting with a gradient of 0-5% methanol/dichloromethane followed by an isocratic gradient of 5% methanol/dichloromethane containing 0.25% ammonium hydroxide to give 2.86 g (75%) of the product as a yellow oil.

¹H NMR (DMSO-d₆) δ ppm: 1.29-1.45 (m, 11H); 1.65-1.79 (m, 1H); 1.93-2.08 (m, 2H); 2.38-2.46 (m, 2H); 2.69-2.79 (m, 1H); 3.10-3.21 (m, 1H); 3.47 (q, 2H); 4.26 (m, 1H) 4.44 (t, 1H); 6.99 (d, 1H).

### Intermediate 71

### Benzyl benzyl-[3-fluoro-1-(2-hydroxyethyl)piperidin-4-yl]carbamate, trans enantiomer A

and

### Intermediate 72

### Benzyl benzyl-[3-fluoro-1-(2-hydroxyethyl)piperidin-4-yl]carbamate, trans enantiomer B

A solution of tetrabutylammonium fluoride in tetrahydrofuran (1M, 21.3 mL, 21.3 mmol) was added to trans (±) benzyl benzyl[1-(2-{[tert-butyl(dimethyl)silyl]oxy}ethyl)-3-fluoropiperidin-4-yl]carbamate (Intermediate 73, 8.9 g, 17.8 mmol) in tetrahydrofuran (20 mL) at 0° C. The solution was allowed to warm to room temperature and stirred for one hour. The mixture was then cooled to 0° C. and quenched with water. The mixture was extracted with ethyl acetate and washed with brine. The organic phase was dried over sodium sulfate and concentrated under reduced pressure. Chromatography on silica with 40% acetone in hexanes gave the product as an oil (5.1 g, 74%).

¹H NMR (DMSO-d₆) δ ppm: 1.55 (m, 1H); 1.67 (m, 1H); 2.02 (m, 2H); 2.40 (m, 2H); 2.74 (m, 1H); 3.14-3.28 (m, 2H); 3.43 (m, 2H); 3.93 (m, 1H); 4.40 (t, 2H); 4.50 (m, 1H); 5.06 (m, 2H); 7.15 (m, 1H); 7.20-7.31 (m, 8H); 7.36 (m, 1H).

MS (ESP): 387.25 (MH⁺) for C₂₂H₂₇FN₂O₃

The racemic mixture was separated on a Chiralpak AD column (500×20 mm, 20 micron) with ethanol/methanol (1:1), containing 0.1% diethyl amine. Trans enantiomer A (Intermediate 71) was eluted first, followed by trans enantiomer B (Intermediate 72). The chiral purity (using an analytical method equivalent to the preparative method described above) was determined to be >98% e.e. for both enantiomers.

### Intermediate 73

### Trans (±) benzyl benzyl-1-(2-{[tert-butyl(dimethyl)silyl]oxy}ethyl)-3-fluoropiperidin-4-yl]carbamate

A mixture of trans (±) benzyl benzyl(3-fluoropiperidin-4-yl)carbamate hydrochloride (Intermediate 74, 7.98 g, 21.1 mmol), (2-bromoethoxy)-tert-butyldimethylsilane (6.85 g, 27.5 mmol) and cesium carbonate (17.9 g, 55.0 mmol) in acetonitrile (60 mL) was heated to 60° C. for twelve hours. The reaction mixture cooled to room temperature and concentrated under reduced pressure to near dryness. The residue was diluted with ethyl acetate and washed with water and brine. The organic phase was dried over sodium sulfate and concentrated under reduced pressure. Chromatography on silica with 10% acetone in hexanes gave the product as oil (8.9 g, 84%).

¹H NMR (chloroform-d₃) δ ppm: 0.04-0.07 (s, 6H); 0.77-0.88 (s, 9H); 1.58-1.74 (m, 2H); 2.05-2.20 (m, 2H); 2.44-2.58 (m, 2H); 2.69-2.84 (m, 1H); 3.24 (m, 1H); 3.65 (s, 2H); 4.44-4.59 (m, 3H); 5.11 (s, 2H); 7.13-7.28 (m, 9H); 7.34 (m, 2H).

MS (ESP): 501.28 (MH⁺) for C₂₈H₄₁FN₂O₃Si

### Intermediate 74

### Trans (±) benzyl benzyl(3-fluoropiperidin-4-yl)carbamate hydrochloride

To a solution of trans (±) tent-butyl 4-{benzyl[(benzyloxy)carbonyl]amino}-3-fluoropiperidine-1-carboxylate (Intermediate 75, 12.05 g, 28 2 mmol) in dichloromethane (50 mL) at 0° C. was added hydrogen chloride (1M in diethyl ether, 56.5 mL, 56.5 mmol). The solution was allowed to stir for one hour. The solid was filtered, and the filter cake washed with diethyl ether to give the mono hydrochloride salt of the product (10.1 g, 95%).

¹H NMR (DMSO-d₆) δ ppm: 1.68 (m, 1H); 2.00-2.15 (m, 1H); 3.08 (m, 1H); 3.18 (m, 1H); 3.34 (m, 2H); 3.50(m, 1H); 4.34-4.49 (m, 2H); 4.65 (m, 1H); 5.02 (s, 1H); 5.14 (d, J=19.40 Hz, 2H); 7.15-7.30 (m, 8H); 7.32 (m, 2H).

MS (ESP): 343.19 (MH⁺) for C₂₀H₂₃FN₂O₂

### Intermediate 75

### Trans (±) tert-butyl-4-{benzyl[(benzyloxy)carbonyl]amino}-3-fluoropiperidine-1-carboxylate

To a solution of trans (±) tert-butyl (4-benzylamino)-3-fluoropiperidine-1-carboxylate (Intermediate 76, 10.3 g, 33.4 mmol) in 1,4-dioxane (100 mL) and sodium carbonate (5.31 g, 50.1 mmol) in water (20 mL) was added benzyl chloroformate (5.89 mL, 41.8 mmol) dropwise at 0° C. The mixture was allowed to warm to room temperature and stirred for two hours. The reaction mixture was then concentrated to near dryness and diluted with ethyl acetate. The organic phase was washed with water and brine, then dried over sodium sulfate. Chromatography on silica with 20% ethyl acetate in hexanes gave the product as a solid (12.5 g, 94%).

MS (ESP): 343.18 (MH⁺minus BOC) for C₂₅H₃₁FN₂O₄

¹H NMR (chloroform-d₃) δ ppm: 1.45 (s, 9H); 1.67 (d, J=8.67 Hz, 2H); 1.84 (m, 1H); 2.59-2.75 (m, 2H); 3.91-4.07 (m, 2H); 4.48 (d, J=16 Hz, 2H); 4.63 (d, J=16 Hz, 1H); 5.18 (s, 2H); 7.20-7.34 (m, 10H).

### Intermediate 76

### Trans (±) tert-butyl (4-benzylamino)-3-fluoropiperidine-1-carboxylate

The title compound was prepared as described by Monique B. van Neil et al, J. Med. Chem., 1999, 42, 2087-2104 and the references therein.

¹H NMR (DMSO-d₆) δ ppm: 1.32 (m, 1H); 1.39 (s, 9H); 1.79 (m, 1H); 2.38 (m, 1H); 2.73 (m, 1H); 3.18 (m, 1H); 3.31 (m, 1H); 3.46 (m, 1H); 3.60-3.80 (m, 3H); 4.39 (m, 1H); 7.20-7.38 (m, 5H).

### Intermediate 77

### 1-{2-[4-Amino-3-fluoropiperidin-1-yl]ethyl}-7-methoxyquinoxalin-2(1H)-one, trans enantiomer A

tert-Butyl {3-fluoro-1-[2-(7-methoxy-2-oxoquinoxalin-1(2H)-yl)ethyl]piperidin-4-yl}carbamate, trans enantiomer A (Intermediate 78, 0.30 g, 0.71 mmol) was reacted with trifluoroacetic acid using a procedure similar to the one described for the synthesis of Intermediate 67 to give 0.25 g of the crude product as an oil.

MS (ESP): 321 (MH⁺) for C₁₆H₂₁FN₄O₂

### Intermediate 78

### tert-Butyl {3-fluoro-1-[2-(7-methoxy-2-oxoquinoxalin-1(2H)-yl)ethyl]piperidin-4-yl}carbamate, trans enantiomer A

7-Methoxyquinoxalin-2(1H)-one (Intermediate 15, 0.52 g, 2.95 mmol), 2-{4-[(tert-butoxycarbonyl)amino]-3-fluoropiperidin-1-yl}ethyl methanesulfonate, trans enantiomer A (Intermediate 69, ˜0.38 mmol/mL, 3.82 mmol), and sodium hydride (60% in oil, 153 mg, 3.82 mmol) were reacted using a procedure similar to the one described for the synthesis of Intermediate 68. Chromatography on silica gel with a gradient of 10-50% acetone in hexanes gave 0.83 g (67%) of the product as an off white solid.

¹H NMR (DMSO-d₆) δ ppm: 1.23-1.45 (m, 11H); 1.64-1.80 (m, 1H); 2.04-2.19 (m, 2H); 2.61-2.71 (m, 2H); 2.84 (d, 1H); 3.25-3.33 (m, 1H); 3.92 (s, 3H); 4.27-4.43 (m, 2H); 4.28 (m, 1H); 16.94-7.05 (m, 3H); 7.75 (d, 1H); 8.04 (s, 1H).

MS (ESP): 421 (MH⁺) for C₂₁H₂₉FN₄O₄

### Intermediate 79

### 1-{2-[4-Amino-3-fluoropiperidin-1-yl]ethyl}-2-oxo-1,2-dihydroquinoline-7-carbonitrile, trans enantiomer B

tert-Butyl {1-[2-(7-cyano-2-oxoquinolin-1(2H)-yl)ethyl]-3-fluoropiperidin-4-yl}carbamate, trans enantiomer B (Intermediate 80, 0.30 g, 0.72 mmol) was reacted with trifluoroacetic acid using a procedure similar to the one described for the synthesis of Intermediate 67 to give 0.25 g of the crude product as an oil.

MS (ESP): 315 (MH⁺) for C₁₇H₁₉FN₄O

### Intermediate 80

### tert-Butyl {1-[2-(7-cyano-2-oxoquinolin-1(2H)-yl)ethyl]-3-fluoropiperidin-4-yl}carbamate, trans enantiomer B

2-Oxo-1,2-dihydroquinoline-7-carbonitrile (Intermediate 5, 0.50 g, 2.94 mmol), 2-{4-[(tert-butoxycarbonyl)amino]-3-fluoropiperidin-1-yl}ethyl methanesulfonate, trans enantiomer B (Intermediate 81, ˜0.38 mmol/mL, 3.82 mmol), and sodium hydride (60% in oil, 153 mg, 3.82 mmol) were reacted using a procedure similar to the one described for the synthesis of Intermediate 68. Chromatography on silica gel with a gradient of 10-50% acetone in hexanes gave 0.64 g (53%) of the product as an off-white solid.

MS (ESP): 415 (MH⁺) for C₂₂H₂₇FN₄O₃

¹H (DMSO-d₆) δ ppm: 1.25-1.43 (m, 11H); 1.67-1.78 (m, 1H); 2.04-2.17 (m, 2H); 2.57-2.68 (m, 2H); 2.80-2.89 (m, 1H); 3.25-3.32 (m, 1H); 4.27-4.47 (m, 2H); 4.30 (m, 1H); 6.78 (d, 1H); 6.99 (d, 1H); 7.66 (dd, 1H); 7.91 (d, 1H); 8.01 (d, 1H); 8.09 (s, 1H).

### Intermediate 81

### 2-{4-[(tert-Butoxycarbonyl)amino]-3-fluoropiperidin-1-yl}ethyl methanesulfonate, trans enantiomer B

tert-Butyl [3-fluoro-1-(2-hydroxyethyl)piperidin-4-yl]carbamate, trans enantiomer B (Intermediate 82, 2.0 g, 7.62 mmol), triethylamine (1.5 mL, 10.7 mmol), and methanesulfonyl chloride (0.71 mL, 9.15 mmol) were reacted using a procedure similar to the one described for the synthesis of Intermediate 69. The crude product was used directly in the next step without further purification.

### Intermediate 82

### tert-Butyl [3-fluoro-1-(2-hydroxyethyl)piperidin-4-yl]carbamate, trans enantiomer B

Benzyl benzyl[3-fluoro-1-(2-hydroxyethyppiperidin-4-yl]carbamate, trans enantiomer B (Intermediate 72, 5.6 g, 14.4 mmol), 20% palladium hydroxide on carbon (0.5 g), and di-tert-butyl dicarbonate (3.5 g, 15.8 mmol) were reacted using a procedure similar to the one described for the synthesis of Intermediate 70. Chromatography on silica gel eluting with 10% methanol (0.1% ammonium hydroxide) in ethyl acetate gave 2.9 g (76%) of product as an oil.

1H NMR (CDCl₃) δ ppm 1.36-1.55 (m, 10H); 2.02-2.31 (m, 3H); 2.52-2.64 (m, 2H); 2.72-2.82 (m, 2H); 3.09-3.20 (m, 1H); 3.60 (t, 311); 4.31 (m, 1H); 4.80 (d, 1H)

### Intermediate 83

### 1-{2-[4-Amino-3-fluoropiperidin-1-yl]ethyl}-7-methoxyquinoxalin-2(1H)-one, trans enantiomer B

tert-Butyl {3-fluoro-1-[2-(7-methoxy-2-oxoquinoxalin-1(2H)-yl)ethyl]piperidin-4-yl}carbamate, trans enantiomer B (Intermediate 84, 0.33 g, 0.78 mmol) was reacted with trifluoroacetic acid using a procedure similar to the one described for the synthesis of Intermediate 67 to give 0.27 g of the crude product as an oil.

MS (ESP): 321 (MH⁺) for C₁₆H₂₁FN₄O₂

### Intermediate 84

### tert-Butyl {3-fluoro-1-[2-(7-methoxy-2-oxoquinoxalin-1(2H)-yl)ethyl]piperidin-4-yl}carbamate, trans enantiomer B

7-Methoxyquinoxalin-2(1H)-one (Intermediate 15, 0.52 g, 2.95 mmol), 2-{4-[(tert-butoxycarbonyl)amino]-3-fluoropiperidin-1-yl}ethyl methanesulfonate, trans enantiomer B (Intermediate 81, ˜0.38 mmol/mL, 3.82 mmol), and sodium hydride (60% in oil, 153 mg, 3.82 mmol) were reacted using a procedure similar to the one described for the synthesis of Intermediate 68. Chromatography on silica gel with a gradient of 10-50% acetone in hexanes gave 0.93 g (78%) of the product as an off white solid.

MS (ESP): 421 (MH⁺) for C₂₁H₂₉FN₄O₄

¹H NMR (DMSO-d₆) δ ppm: 1.23-1.45 (m, 11H); 1.64-1.80 (m, 1H); 2.04-2.19 (m, 2H); 2.61-2.71 (m, 2H); 2.84 (d, 1H); 3.25-3.33 (m, 1H); 3.92 (s, 3H); 4.27-4.43 (m, 2H); 4.28 (m, 1H); 16.94-7.05 (m, 3H); 7.75 (d, 1H); 8.04 (s, 1H).

### Intermediate 85

### 1-{2-[(3R,4R)-4-Amino-3-methoxypiperidin-1-yl]ethyl}-2-oxo-1,2-dihydroquinoline-7-carbonitrile

A solution of tert-butyl {(3R,4R)-1-[2-(7-cyano-2-oxoquinolin-1(2H)-yl)ethyl]-3-methoxypiperidin-4-yl}carbamate (Intermediate 86, 280 mg, 0.66 mmol) in dichloromethane (30 mL) was treated with trifluoroacetic acid (10 mL). After 1 hour, the reaction was concentrated to dryness. The residue was taken up in 15% methanol in chloroform (30 mL) and washed with saturated sodium bicarbonate solution. The aqueous layer was adjusted to pH ˜10 with saturated sodium carbonate solution, and re-extracted with 15% methanol/chloroform (3×30 mL). The combined organic phases were dried over sodium sulfate and concentrated to dryness giving 240 mg of the crude product as an oil.

MS (ESP): 327 (MH⁺) for C₁₈H₂₂N₄O₂

### Intermediate 86

### tert-Butyl {(3R,4R)-1-[2-(7-cyano-2-oxoquinolin-1(2H)-yl)ethyl]-3-methoxypiperidin-4-yl}carbamate

and

### Intermediate 87

### tert-Butyl {(3S,4S)-1-[2-(7-cyano-2-oxoquinolin-1(2H)-yl)ethyl]-3-methoxypiperidin-4-yl}carbamate

A mixture of tert-butyl [trans(±)-3-methoxypiperidin-4-yl]carbamate (Intermediate 53, 0.63 g, 2.7 mmol) and 2-oxo-1-(2-oxoethyl)-1,2-dihydroquinoline-7-carbonitrile (Intermediate 88, 0.57 g, 2.7 mmol) in 1:1 dry methanol/chloroform (30 mL) was heated to 70° C. for 2 hours. The reaction was cooled to room temperature, treated with sodium triacetoxy borohydride (1.7 g, 8.1 mmol) and stirred at room temperature for 2 hours. The reaction was filtered through celite, and the filtrate was concentrated in vacuo. The crude residue was partitioned between ethyl acetate and saturated aqueous sodium bicarbonate. The layers were separated and the aqueous phase was re-extracted once with ethyl acetate. The combined organic phases were dried over sodium sulfate, filtered, and concentrated in vacuo. Chromatography on silica gel with 25-50% acetone in hexanes gave 0.74 g (62%) of the racemic mixture of the products.

MS (ESP): 427 (MH⁺) for C₂₃H₃₀N₄O₄

¹H NMR (DMSO-d₆) δ ppm: 1.19-1.33 (m, 1H); 1.37 (s, 9H); 1.64-1.73 (m, 1H); 1.77 (m, 1H); 1.99 (m, 1H); 2.59 (m, 2H); 2.79-2.87 (m, 1H); 2.93-3.04 (m, 1H); 3.05-3.15 (m, 1H); 3.23-3.30 (m, 1H); 3.28 (s, 3H); 4.30-4.47 (m, 2H); 6.79 (d, 2H); 7.66 (dd, 1H); 7.91 (d, 1H); 8.01 (d, 1H); 8.09 (s, 1H).

The mixture of enantiomers was separated by HPLC on a Chiralpak AD column (20×250 mm, 10 micron) with an isocratic gradient of 80% hexanes, 20% 1:1 ethanoUmethanol, 0.1% diethylamine at a flow rate of 20 mL/min. This gave 0.28 g of tert-butyl {(3R,4R)-1-[2-(7-cyano-2-oxoquinolin-1(2H)-yl)ethyl]-3-methoxypiperidin-4-yl}carbamate (Intermediate 86, second eluting peak, (+) isomer) and 0.32 g of tert-butyl {(3S,4S)-1-[2-(7-cyano-2-oxoquinolin-1(2H)-yl)ethyl]-3-methoxypiperidin-4-yl}carbamate (Intermediate 87, first eluting peak, (−) isomer).

### Intermediate 88

### 2-Oxo-1-(2-oxoethyl)-1,2-dihydroquinoline-7-carbonitrile

To a solution of 1-(2,2-diethoxyethyl)-2-oxo-1,2-dihydroquinoline-7-carbonitrile (Intermediate 89, 21.5 g, 75.1 mmol) in acetonitrile (230 mL) was added concentrated hydrochloric acid (2 eq., 12.5 mL). After 1 hour, the resulting precipitate was collected by filtration. This gave 16 g (100%) of the product as a colorless solid after drying, which was used without further purification.

MS (ESP): 213 (MH⁺) for C₁₂H₈N₂O₂

¹H NMR (DMSO-d₆) δ ppm: 5.25-5.38 (m, 2H); 6.82 (d, 1H); 7.67 (d, 1H); 7.95 (d, 1H); 8.02-8.14 (m, 2H); 9.64-9.74 (m, 1H).

### Intermediate 89

### 1-(2,2-Diethoxyethyl)-2-oxo-1,2-dihydroquinoline-7-carbonitrile

A mixture of 2-oxo-1,2-dihydroquinoline-7-carbonitrile (Intermediate 5, 35.0 g, 201 mmol), 2-bromo-1,1-diethoxyethane (44.1 mL, 281 mmol) and cesium carbonate (78.5 g, 241 mmol) in dry NMP (200 mL) was stirred at 70° C. overnight. The reaction mixture was diluted with water (350 mL) and extracted with butyl acetate (2×350 mL). The combined organic phases were filtered through celite and washed with water (1×175 mL). The butyl acetate solution was concentrated to 140 mL and diluted with iso-hexane 525 ml. The precipitate was isolated by filtration and washed with iso-hexane 70 ml. This gave 34 g (60%) of the product as a colorless solid after drying, which was used without further purification.

MS (ESP): 309 (MNa⁺) for C₁₆H₁₈N₂O₃

¹H-NMR (DMSO-d₆) δ ppm: 0.96 (t, 6H); 3.34-3.47 (m, 2H); 3.56-3.73 (m, 2H); 4.39 (d, 2H); 4.72 (t, 1H); 6.80 (d, 1H); 7.62 (d, 1H); 7.89 (d, 1H); 8.02 (d, 1H); 8.13-8.22 (m, 1H).

### Intermediate 90

### 1-{2-[(3S,4S)-4-Amino-3-methoxypiperidin-1-yl]ethyl}-2-oxo-1,2-dihydroquinoline-7-carbonitrile

tert-Butyl {(3S,4S)-1-[2-(7-cyano-2-oxoquinolin-1(2H)-yl)ethyl]-3-methoxypiperidin-4-yl}carbamate (Intermediate 87, 0.32 g, 0.75 mmol) was reacted with trifluoroacetic acid using a procedure similar to the one described for the synthesis of Intermediate 85 to give 0.24 g of the crude product as an oil.

MS (ESP): 327 (MH⁺) for C₁₈H₂₂N₄O₂

### Intermediate 91

### Cis(±) 1-[2-(4-amino-3-fluoropiperidin-1-yl)ethyl]-7-methoxyquinoxalin-2(1H)-one, trifluoroacetic acid salt

To a solution of cis(±) tert-butyl {3-fluoro-1-[2-(7-methoxy-2-oxoquinoxalin-1(2H)-yl)ethyl]piperidin-4-yl}carbamate (Intermediate 92, 222 mg, 0.53 mmol), in chloroform (10 mL) at 0° C., was added 30% trifluoroacetic acid in chloroform (6 mL). After 3 hours at room temperature the solvent was removed under reduced pressure, providing the title compound, which was used in the next step without purification. The title compound may be present in the form of a bis-trifluoroacetic acid salt.

MS (ESP): 321 (MH⁺) for C₁₆H₂₁FN₄O

### Intermediate 92

### Cis(±) tert-butyl {3-fluoro-1-[2-(7-methoxy-2-oxoquinoxalin-1(2H)-yl)ethyl]piperidin-4-yl}carbamate

7-Methoxyquinoxalin-2(1H)-one (Intermediate 15, 181 mg, 1 mmol) and cis(±) 2-{4-[(tert-butoxycarbonyl)amino]-3-fluoropiperidin-1-yl}ethyl methanesulfonate (Intermediate 21, ˜1 mmol) were reacted using a procedure similar to the one described for the synthesis of Intermediate 20. Chromatography on silica gel with hexanes/ethyl acetate (2:3) gave 222 mg (51%) product as a solid.

MS (ESP): 421 (MH⁺) for C₂₁H₂₉FN₄O₄

¹H-NMR (CDCl₃-d) δ: 1.44 (s, 9H); 1.86 (m, 2H); 2.40 (m, 2H); 2.80 (m, 2H); 3.15 (m, 1H); 3.41 (m, 1H); 3.70 (m, 1H); 3.94 (s, 3H); 4.42 (m, 2H); 4.70 (m, 2H); 6.93 (m, 2H); 7.77 (m, 1H); 8.11 (s, 1H).

### Intermediate 93

### 1-{2-[(3R,4S)-4-Amino-3-methoxyniperidin-1-yl]ethyl}-4-methyl-2-oxo-1,2-dihydroquinoline-7-carbonitrile, trifluoroacetic acid salt

Tert-butyl {(3R,4S)-1-[2-(7-cyano-4-methyl-2-oxoquinolin-1(2H)-yl)ethyl]-3-methoxypiperidin-4-yl}carbamate (Intermediate 94, 360 mg, 0.81 mmol) was reacted with trifluoroacetic acid using a procedure similar to the one described for the synthesis of Intermediate 91, providing the title compound. The title compound may be present in the form of a bis trifluoroacetic acid salt.

MS (ESP): 341 (MH⁺) for C₁₉H₂₄N₄O₂

### Intermediate 94

### tert-Butyl {(3R,4S)-1-[2-(7-cyano-4-methyl-2-oxoquinolin-1(2H)-yl)ethyl]-3-methoxypiperidin-4-yl}carbamate

To a solution of 4-methyl-2-oxo-1,2-dihydroquinoline-7-carbonitrile (V. N. Gogte, S. B. Kulkarni and B. D. Tilak, Indian Journal of Chemistry, 15B, 769-773 (1977)) (368 mg, 2 mmol) in anhydrous DMF (20 mL) was added sodium hydride (120 mg, 60% in oil, 3 mmol). The mixture was stirred for 1 hour at room temperature. A solution of 2-{(3R,4S)-4-[(tert-butoxycarbonyl)amino]-3-methoxypiperidin-1-yl}ethyl methanesulfonate (Intermediate 39, ˜2 mmol) in DMF was then added at 0° C. The reaction mixture was stirred overnight at room temperature, and was then diluted with water and extracted three times with dichloromethane. The organic extracts were dried over magnesium sulfate and concentrated. Silica gel chromatography with hexanes/ethyl acetate (3:2) afforded 360 mg of a 3:1 mixture of the title compound and the O-alkylation product.

MS (ESP): 441 (MH⁺) for C₂₄H₃₂N₄O₄

### Intermediate 95

### Cis (±)1-[2-(4-amino-3-fluoropiperidin-1-yl)ethyl]-4-methyl-2-oxo-1,2-dihydroquinoline-7-carbonitrile, trifluoroacetic acid salt

Cis(±) tert-butyl {1-[2-(7-cyano-4-methyl-2-oxoquinolin-1(2H)-yl)ethyl]-3-fluoropiperidin-4-yl}carbamate (Intermediate 96, 269 mg, 0.63 mmol) was reacted with trifluoroacetic acid using a procedure similar to the one described for the synthesis of Intermediate 91 to give the crude product (200 mg). The title compound may be present in the form of a bis-trifluoroacetic acid salt.

MS (ESP): 329 (MH⁺) for C₁₈H₂₁FN₄O

### Intermediate 96

### Cis(±) tert-butyl {1-[2-(7-cyano-4-methyl-2-oxoquinolin-1(2H)-yl)ethyl]-3-fluoropiperidin-4-yl}carbamate

To a mixture of 4-methyl-2-oxo-1,2-dihydroquinoline-7-carbonitrile (V. N. Gogte, S. B. Kulkarni and B. D. Tilak, Indian Journal of Chemistry, 15B, 769-773 (1977)) (350 mg, 1.9 mmol) in anhydrous DMF (20 mL) was added sodium hydride (90 mg, 60% in oil, 2.2 mmol). The mixture was stirred for 1 hour at room temperature. A solution of cis(±) 2-{4-[(tert-butoxycarbonyl)amino]-3-fluoropiperidin-1-yl}ethyl methanesulfonate (Intermediate 21, ˜1.9 mmol) in DMF was then added at 0° C. The reaction mixture was stirred overnight at room temperature, and was then diluted with water and extracted three times with dichloromethane. The organic extracts were dried over magnesium sulfate and concentrated. Silica gel chromatography with hexanes/ethyl acetate (1:1) afforded 269 mg (33%) of the product as a tan solid.

MS (ESP): 428 (MH⁺) for C₂₃H₂₉FN₄O₃

### Intermediate 97

### 1-(2-((3S,4R)-4-Amino-3-fluoropiperidin-1-yl)ethyl)-2-oxo-1,2-dihydroquinoline-7-carbonitrile, trifluoroacetic acid salt

tert-Butyl (3S,4R)-1-(2-(7-cyano-2-oxoquinolin-1(2H)-yl)ethyl)-3-fluoropiperidin-4-ylcarbamate (Intermediate 98, 0.5 g, 1.21 mmol) was reacted with trifluoroacetic acid using a procedure similar to the one described for the synthesis of Intermediate 91 to give the crude product in quantitative yield, which was used directly for next step. The title compound may be present in the form of a bis-trifluoroacetic acid salt.

MS (ESP): 315 (MH⁺) for C₁₇F₁₉FN₄O

### Intermediate 98

### tert-Butyl (3S,4R)-1-(2-(7-cyano-2-oxoquinolin-1(2H)-yl)ethyl)-3-fluoropiperidin-4-ylcarbamate

A mixture of 2-oxo-1,2-dihydroquinoline-7-carbonitrile (Intermediate 5, 0.5 g, 2.94 mmol) and 60 wt. % sodium hydride in mineral oil (0.176 g, 4.41 mmol) in DMF (50 mL) was stirred at room temperature under nitrogen for 1 hour. The solution was cooled to 0° C. and a solution of 2-((3S,4R)-4-(tert-butoxycarbonylamino)-3-fluoropiperidin-1-yl)ethylmethanesulfonate (Intermediate 99, 1 g, 2.94 mmol) in DMF (10 mL) was added. The reaction mixture was stirred overnight at room temperature. The reaction mixture was diluted with water and extracted twice with dichloromethane. The combined organic extracts were dried over magnesium sulfate, filtered and evaporated. Silica gel chromatography (0%-75% ethyl acetate in hexanes) afforded the title compound as a tan solid, 0.55 g (45%). [α]_{D}=+0.063 (c=0.2, DMSO).

MS (ESP): 415 (MH⁺) for C₂₂H₂₇FN₄O₃

### Intermediate 99

### 2-((3S,4R)-4-(tert-Butoxycarbonylamino)-3-fluoronineridin-1-yl)ethylmethanesulfonate

The title compound was prepared from (3S,4R)-1-(2-(tert-butyldimethylsilyloxy)ethyl)-3-fluoropiperidin-4-amine (Intermediate 100) using a procedure similar to the one described for the synthesis of Intermediate 21 from Intermediate 24.

MS (ESP): 341 (MH⁺) for C₁₃H₂₅FN₂O₅S

### Intermediate 100

### (3S,4R)-1-(2-tert-Butyldimethylsilyloxy)ethyl)-3-fluoropiperidin-4-amine

A solution of benzyl (3S,4R)-1-(2-(tert-butyldimethylsilyloxy)ethyl)-3-fluoropiperidin-4-ylcarbamate (Intermediate 101, 8 g, 19.48 mmol) in ethanol (100 ml) was hydrogenated on palladium on carbon (10%, activated, 1.037g) under normal pressure at room temperature overnight. The reaction mixture was filtered through a 0.45 μm membrane and solvent was evaporated under reduced pressure to afford the title compound as an oil (5 g, 93%).

MS (ESP): 277 (MH⁺) for C₁₃H₂₉FN₂OSi

¹H-NMR (CDCl₃-d) δ: 0.04 (s, 6H); 0.87 (s, 9H); 1.75 (m, 4H); 2.35 (m, 2H); 2.56 (m, 2H); 2.81 (m, 2H); 3.15 (m, 1H); 3.74 (m, 2H); 4.57 (m, 1H).

### Intermediate 101

### Benzyl (3 S,4R)-1-(2-tert-butyldimethylsilyloxy)ethyl)-3-fluoropiperidin-4-ylcarbamate

To a stirred mixture of benzyl (3S,4R)-3-fluoropiperidin-4-ylcarbamate hydrochloride (Intermediate 102, 5.9 g, 20.43 mmol) and cesium carbonate (33.3 g, 102.17 mmol) in acetonitrile (300 mL) at room temperature was added (2-bromoethoxy)(tert-butyl)dimethylsilane (21.92 mL, 102.17 mmol). The reaction was stirred at 60° C. overnight. The reaction mixture was filtered through a fritted funnel and concentrated. Silica gel chromatography (0%-50% ethyl acetate in hexanes) afforded the title compound as a yellow oil, 8 g (95%).

MS (ESP): 411 (MH⁺) for C₂₁H₃₅FN₂O₃Si

### Intermediate 102

### Benzyl (3S,4R)-3-fluoropiperidin-4-ylcarbamate, hydrochloride salt

To solution of (3 S,4R)-tert-butyl 4-(benzyloxycarbonylamino)-3-fluoropiperidine-1-carboxylate (Intermediate 103, 8 g, 22.7 mmol) in dichloromethane (200 mL) at 0° C. was added 4M hydrogen chloride in dioxane (11.35 mL, 45.4 mmol). The reaction mixture was allowed to warm to room temperature and stir overnight. Another equivalent of 4M hydrogen chloride in dioxane was added and the reaction was stirred for another 4 hours. The resulting white precipitate was collected by filtration and dried under reduced pressure to give the title compound (5.9 g, 90%).

MS (ESP): 253 (MH⁺) for C₁₃H₁₇FN₂O₂

### Intermediate 103

### (3 S,4R)-tert-Butyl 4-(benzyloxycarbonylamino)-3-fluoropiperidine-1-carboxylate

To a mixture of (3S,4R)-tert-butyl 4-amino-3-fluoropiperidine-1-carboxylate (prepared using the procedures described in PCT Pub. Nos. WO 2006087543 and WO2007071965) (5.1 g, 23.37 mmol) in dioxane (150 ml) and saturated sodium carbonate (50 mL) at 0° C. was added benzyl chloroformate (5.00 ml, 35.05 mmol). After 15 minutes, the reaction mixture was diluted with ethyl acetate and saturated sodium chloride. The layers were separated and the organic extracts were dried over magnesium sulfate, filtered and evaporated. Silica gel chromatography (0%-50% ethyl acetate in hexanes) afforded the title compound as an off-white solid, 8 g (97%).

MS (ESP): 353 (MH⁺) for C₁₈H₂₅FN₂O₄

¹H-NMR (CDCl₃-d) δ: 1.44 (m, 9H); 1.73 (m, 2H); 2.80 (m, 2H); 3.60 (m, 1H); 4.30 (m, 2H); 4.65 (m, 1H); 5.06 (m, 1H); 5.09 (s, 2H); 7.34 (m, 5H).

### Intermediate 104

### 1-{2-[(3S,4R)-4-Amino-3-methoxypiperidin-1-yl]ethyl}-4-methyl-2-oxo-1,2-dihydroquinoline-7-carbonitrile, trifluoroacetic acid salt

tert-Butyl {(3S,4R)-1-[2-(7-cyano-4-methyl-2-oxoquinolin-1(2H)-yl)ethyl]-3-methoxypiperidin-4-yl}carbamate (Intermediate 105, 191 mg, 0.43 mmol) was reacted with trifluoroacetic acid using a procedure similar to the one described for the synthesis of Intermediate 91 to give the crude title compound in quantitative yield, which was used directly for next step. The title compound may be present in the form of the bis-trifluoroacetic acid salt.

MS (ESP): 341 (MH⁺) for C₁₉H₂₄N₄O₂

### Intermediate 105

### tert-Butyl {(3S,4R)-1-[2-(7-cyano-4-methyl-2-oxoquinolin-1(2H)-yl)ethyl]-3-methoxypiperidin-4-yl}carbamate

To a solution of 4-methyl-2-oxo-1,2-dihydroquinoline-7-carbonitrile (V. N. Gogte, S. B. Kulkarni and B. D. Tilak, Indian Journal of Chemistry, 15B, 769-773 (1977)) (435 mg, 2.36 mmol) in anhydrous DMF (20 mL) was added sodium hydride (142 mg, 60% in oil, 3.54 mmol). The mixture was stirred for 1 hour at room temperature. A solution of 2-{(3S,4R)-4-[(tert-butoxycarbonyl)amino]-3-methoxypiperidin-1-yl}ethyl methanesulfonate (Intermediate 33, ˜2.8 mmol) in DMF was then added at 0° C. The reaction mixture was stirred overnight at room temperature, and was then diluted with water, and extracted three times with dichloromethane. The organic extracts were dried over magnesium sulfate and concentrated. Reverse phase chromatography with acetonitrile/water/ammonium acetate afforded the title compound as a light yellow solid after lyophilization (191 mg, 18%).

MS (ESP): 441 (MH⁺) for C₂₄H₃₂N₄O₄

### Intermediate 106

### 1-(2-((3R,4S)-4-Amino-3-fluoropiperidin-1-yl)ethyl)-2-oxo-1,2-dihydroquinoline-7-carbonitrile trifluoroacetate

The title compound was prepared from tert-butyl (3R,4S)-4-(benzylamino)-3-fluoropiperidine-1-carboxylate (WO2007071965 and WO2006087543) by a process analogous to the sequence described for the racemic material (Intermediate 19), except for the following modification in the final step: The crude trifluoroacetic acid salt of the title compound was taken up in dichloromethane (100 mL) and washed once with saturated sodium bicarbonate (20 mL). The aqueous wash was adjusted to pH=10 with 20% sodium hydroxide and extracted three times with dichloromethane (100 mL). The combined organic extracts were dried over magnesium sulfate, filtered and concentrated to dryness to afford the title compound as an off-white solid, 352 mg (81%).

MS (ESP): 315 (MH⁺) for C₁₇H₁₉FN₄O

### Example 1

### 1-(2-{(3R,4S)-4-[(2,3-Dihydro[1,4]dioxino[2,3-c]pyridin-7-ylmethyl)amino]-3-hydroxypiperidin-1-yl}ethyl)-2-oxo-1,2-dihydroquinoline-7-carbonitrile, bis hydrochloride salt

A mixture of 1-{2-[(3R,4S)-4-amino-3-hydroxypiperidin-1-yl]ethyl}-2-oxo-1,2-dihydroquinoline-7-carbonitrile (Intermediate 1, 74 mg, 0.24 mmol) and 2,3-dihydro[1,4]dioxino[2,3-c]pyridine-7-carbaldehyde (WO 2004/058144) (39 mg, 0.24 mmol) was heated over freshly activated 3 Å molecular sieves at 70° C. for 3 hours. The reaction mixture was cooled to room temperature, and sodium triacetoxy borohydride (150 mg, 0.75 mmol) was added. The resulting reaction mixture was stirred at room temperature for 30 minutes and then was filtered through a 0.45 μm membrane and concentrated to dryness under reduced pressure. Chromatography on silica gel with dichloromethane/methanol (8:1 to 4:1). Fractions containing product were pooled and concentrated to dryness. The residue was taken up in dichloromethane/diethyl ether (1:2, 10 mL) and HCl in diethyl ether (2M, ˜0.15 mL) was added. The mixture was concentrated to dryness under reduced pressure, codistilled two times with dichloromethane (2×15 mL) and titurated from ether to give the title composition as a colorless solid, 91 mg (72%), mp>210° C.

MS (ESP): 462 (MH⁺) for C₂₅H₂₇N₅O₄

¹H-NMR (DMSO-d₆) δ: 2.18 (m, 2H); 3.15 (m, 1H); 3.25-3.36 (m, 4H); 3.69 (m, 2H); 4.10-4.49 (m, 7H); 4.61 (dd, 2H); 6.64 (brs, 1H); 6.83 (d, 1H); 7.30 (s, 1H); 7.72 (d, 1H); 7.97 (d, 1H); 8.08 (d, 1H); 8.22 (m, 2H); 9.45 (brs, 2H); 10.00 (brs, 1H).

### Example 2

### 1-[2-((3R,4S)-3-Hydroxy-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile, bis hydrochloride salt

3-Oxo-3,4-dihydro-2H-pyrido[3,2-b][1,4]oxazine-6-carbaldehyde (WO 2004/058144) and Intermediate 1 were reacted using a procedure similar to the one described for the synthesis of Example 1, providing the title composition.

¹H NMR (D₂O) δ: (D₂O) 2.28 (m, 2H); 3.15 (ddd, 1H); 3.26 (m, 1H); 3.40-3.72 (m, 4H); 3.91 (m, 1H); 4.28 (s, 2H); 4.52 (m, 1H); 4.61 (m, 1H); 4.71 (s, 2H); 4.80 (m, 1H); 6.65 (d, 1H); 7.08 (d, 1H); 7.35 (d, 1H); 7.66 (d, 1H); 7.87 (d, 1H); 7.96 (s, 1H); 8.02 (d, 1H).

ES (MH)^{±}: 475

### Example 3

### 1-[2-((3R,4S)-3-Hydroxy-4-{[(3-oxo-3,4-dihydro-2H-pyrido[3,2-b][1,4]thiazin-6-yl)methyl]amino}piperidin-1-yl)ethyl]-2-oxo-1,2-dihydroquinoline-7-carbonitrile, bis-hydrochloride salt

3-Oxo-3,4-dihydro-2H-pyrido[3,2-b][1,4]thiazine-6-carbaldehyde (WO 2004/058144) and Intermediate 1 were reacted using a procedure similar to the one described for the synthesis of Example 1, providing the title composition.

¹H NMR (D₂O) δ: (D₂O) 2.31 (m, 2H); 3.20 (ddd, 1H); 3.31 (m, 1H); 3.40-3.80 (m, 4H); 3.55 (s, 2H); 3.95 (m, 1H); 4.34 (s, 2H); 4.55-4.70 (m, 2H); 4.82 (m, 1H); 6.84 (d, 1H); 7.11 (d, 1H); 7.66 (d, 1H); 7.80 (d, 1H); 7.87 (d, 1H); 7.96 (s, 1H); 8.03 (d, 1H).

ES (MH)⁺: 491

### Example 4

### 1-(2-{(3S,4R)-4-[(2,3-Dihydro[1,4]dioxino[2,3-c]pyridin-7-ylmethyl)amino]-3-hydroxypiperidin-1-yl}ethyl)-2-oxo-1,2-dihydroquinoline-7-carbonitrile, bis hydrochloride salt

2,3-Dihydro[1,4]dioxino[2,3-c]pyridine-7-carbaldehyde and Intermediate 2 were reacted using a procedure similar to the one described for the synthesis of Example 1, providing the title composition.

¹H-NMR (DMSO-d₆) δ: 2.17 (m, 2H); 3.14 (m, 1H); 3.25-3.38 (m, 4H); 3.69 (m, 2H); 4.00-4.49 (m, 7H); 4.61 (dd, 2H); 6.62(brs, 1H); 6.83 (d, 1H); 7.25 (s, 1H); 7.72 (d, 1H); 7.97 (d, 1H); 8.06 (d, 1H); 8.20 (m, 2H); 9.39 (brs, 2H); 9.93 (brs, 1H).

ES (MH)⁺: 462

### Example 5

### 1-[2-((3S,4R)-3-Hydroxy-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile, bis hydrochloride salt

3-Oxo-3,4-dihydro-2H-pyrido[3,2-b][1,4]oxazine-6-carbaldehyde and Intermediate 2 were reacted using a procedure similar to the one described for the synthesis of Example 1, providing the title composition.

¹H NMR (DMSO-d₆) δ: 2.20 (m, 2H); 3.19 (m, 1H); 3.25-3.48 (m, 4H); 3.71 (m, 2H); 4.19 (m, 2H); 4.55-4.65 (m, 3H); 4.69 (s, 2H); 6.63 (brs, 1H); 6.83 (d, 1H); 7.26 (d, 1H); 7.44 (d, 1H); 7.73 (d, 1H); 7.97 (d, 1H); 8.07 (d, 1H); 8.24 (brs, 1H); 9.28 (m, 1H); 9.58 (m, 1H); 10.07 (m, 1H); 11.40 (m, 1H).

ES (MH)⁺: 475

### Example 6

### 1-[2-((3S,4R)-3-Hydroxy-4-{[(3-oxo-3,4-dihydro-2H-pyrido[3,2-b][1,4]thiazin-6-yl)methyl]amino}piperidin-1-yl)ethyl]-2-oxo-1,2-dihydroquinoline-7-carbonitrile, bis hydrochloride salt

3-Oxo-3,4-dihydro-2H-pyrido[3,2-b][1,4]thiazine-6-carbaldehyde and Intermediate 2 were reacted using a procedure similar to the one described for the synthesis of Example 1, providing the title composition.

¹H NMR DMSO-d₆) δ: 2.21 (m, 2H); 3.20 m, 1H); 3.30-3.76 (m, 6H); 3.60 (s, 2H); 4.24 (m, 2H); 4.57-4.67 (m, 3H); 6.62 (d, 1H); 6.83 (d, 1H); 7.27 (d, 1H); 7.73 (d, 1H); 7.89 (d, 1H); 7.98 (d, 1H); 8.07 (d, 1H); 8.24 (m, 1H); 9.34 (m, 1H); 9.61 (m, 1H); 10.04 (m, 1H); 11.08 (s, 1H).

ES (MH)⁺: 491

### Example 7

### 1-(2-{(3R,4S)-4-[(2,3-Dihydro[1,4]dioxino[2,3-c]pyridin-7-ylmethyl)amino]-3-hydroxypiperidin-1-yl}ethyl)-7-methoxyquinoxalin-2(1H)-one, bis hydrochloride salt

2,3-Dihydro[1,4]dioxino[2,3-c]pyridine-7-carbaldehyde and Intermediate 11 were reacted using a procedure similar to the one described for the synthesis of Example 1, providing the title composition.

¹H NMR (D₂O) δ: 2.28 (m, 2H); 3.20 (ddd, 1H); 3.31 (m, 1H); 3.45-3.76 (m, 4H); 3.92 (s, 3H); 3.99 (m, 1H); 4.29-4.62 (m, 8H); 4.83 (m, 1H); 6.93 (m, 1H); 7.12 (m, 1H); 7.22 (d, 1H); 7.81 (dd, 1H); 8.06 (d, 1H); 8.19 (d, 1H).

ES (MH)⁺: 468

### Example 8

### 6-[({(3R,4S)-3-Hydroxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one, bis hydrochloride salt

3-oxo-3,4-dihydro-2H-pyrido[3,2-b][1,4]oxazine-6-carbaldehyde and Intermediate 11 were reacted using a procedure similar to the one described for the synthesis of Example 1, providing the title composition.

¹H NMR (DMSO-d₆) δ: 2.19 (m, 2H); 3.19 (m, 1H); 3.30-3.48 (m, 4H); 3.62-3.83 (m, 2H); 3.96 (s, 3H); 4.18 (m, 2H); 4.54-4.76 (m, 3H); 4.69 (s, 2H); 6.54 (m, 1H); 7.04 (dd, 1H); 7.21 (d, 1H); 7.26 (d, 1H); 7.44 (d, 1H); 7.79 (d, 1H); 8.09 (s, 1H); 9.28 (m, 1H); 9.61 (m, 1H); 10.26 (m, 1H); 11.41 (s, 1H).

ES (MH)⁺: 481

### Example 9

### 6-[({(3R,4S)-3-Hydroxy-1-[2-(7-methoxy-2-oxoquinoxalin-1(2H)-yl)ethyl]piperidin-4-yl}amino)methyl]-2H-pyrido[3,2-b][1,4]thiazin-3(4H)-one, bis hydrochloride salt

3-Oxo-3,4-dihydro-2H-pyrido[3,2-b][1,4]thiazine-6-carbaldehyde and Intermediate 11 were reacted using a procedure similar to the one described for the synthesis of Example 1, providing the title composition.

¹H NMR (DMSO-d₆) δ: 2.19 (m, 2H); 3.17 (m, 1H); 3.25-3.70 (m, 6H); 3.59 (s, 2H); 3.96 (s, 3H); 4.23 (m, 2H); 4.55-4.67 (m, 3H); 6.53 (d, 1H); 7.04 (dd, 1H); 7.20 (d, 1H); 7.27 (d, 1H); 7.79 (d, 1H); 7.90 (d, 1H); 8.09 (s, 1H); 9.30 (m, 1H); 9.60 (m, 1H); 10.21 (m, 1H); 11.08 (s, 1H).

ES (MH)⁺: 497

### Example 10

### 1-(2-{(3S,4R)-4-[(2,3-Dihydro[1,4]dioxino[2,3-c]pyridin-7-ylmethyl)amino]-3-hydroxypiperidin-1-yl}ethyl)-7-methoxyquinoxalin-2(1H)-one, bis hydrochloride salt

2,3-Dihydro[1,4]dioxino[2,3-c]pyridine-7-carbaldehyde and Intermediate 12 were reacted using a procedure similar to the one described for the synthesis of Example 1, providing the title composition.

¹H NMR (DMSO-d₆) δ: 2.18 (m, 2H); 3.16 (m, 1H); 3.26-3.40 (m, 4H); 3.65-3.74 (m, 2H); 3.96 (s, 3H); 4.26-4.43 (m, 7H); 4.62 (dd, 2H); 6.55 (m, 1H); 7.04 (dd, 1H); 7.19 (d, 1H); 7.36 (s, 1H); 7.79 (d, 1H); 8.08 (s, 1H); 8.26 (s, 1H); 9.51 (m, 2H); 10.18 (m, 1H).

ES (MH)⁺: 468

### Example 11

### 6-[({(3S,4R)-3-Hydroxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one, bis hydrochloride salt

3-Oxo-3,4-dihydro-2H-pyrido[3,2-b][1,4]oxazine-6-carbaldehyde and Intermediate 12 were reacted using a procedure similar to the one described for the synthesis of Example 1, providing the title composition.

¹H NMR (D₂O) δ: 2.28 (m, 2H); 3.17 (ddd, 1H); 3.27 (m, 1H); 3.51-3.75 (m, 4H); 3.91 (s, 3H); 3.99 (m, 1H); 4.28 (m, 2H); 4.50-4.62 (m, 2H); 4.71 (s, 2H); 4.80 (m, 1H); 6.90 (d, 1H); 7.08 (d, 1H); 7.11 (dd, 1H); 7.34 (d, 1H); 7.79 (d, 1H); 8.05 (s, 1H).

ES (MH)⁺: 481

### Example 12

### 1-(2-{(3S,4R)-4-[(2,3-Dihydro[1,4]dioxino[2,3-c]pyridin-7-ylmethyl)amino]-3-fluoropiperidin-1-yl}ethyl)-2-oxo-1,2-dihydroquinoline-7-carbonitrile, monoacetic acid salt

and

### Example 13

### 1-(2-{(3R,4S)-4-[(2,3-Dihydro[1,4]dioxino[2,3-c]pyridin-7-ylmethyl)amino]-3-fluoropiperidin-1-yl}ethyl)-2-oxo-1,2-dihydroquinoline-7-carbonitrile, monoacetic acid salt

Cis(±) 1-[2-(4-amino-3-fluoropiperidin-1-yl)ethyl]-2-oxo-1,2-dihydroquinoline-7-carbonitrile (Intermediate 19, 0.95 mmol) was suspended in chloroform/methanol (1:1, 20 mL) and free based by dropwise addition of N,N-diisopropylethylamine until all material was solubilized. This was then subjected to reductive amination with 2,3-dihydro[1,4]dioxino[2,3-c]pyridine-7-carbaldehyde (160 mg, 0.95 mmol) and sodium triacetoxyborohydride (600 mg, 2.8 mmol) using a procedure similar to the one described for the synthesis of Example 1. The residue obtained after filtration was taken up in dichloromethane and saturated sodium bicarbonate. The pH of the aqueous phase was adjusted to pH˜10 with 1M sodium hydroxide solution. The aqueous phase was back extracted with dichloromethane and the combined organic phases were dried over sodium sulfate and concentrated. Reverse phase chromatography with water/acetonitrile/ammonium acetate afforded the product as a tan foam, 276 mg (62%).

¹H-NMR (CDCl₃-d) δ: 1.86 (m, 2H); 2.34 (m, 3H); 2.71 (m, 2H); 2.82 (m, 1H); 3.03 (m, 1H); 3.30 (m, 1H); 3.93 (m, 2H); 4.30 (m, 4H); 4.42 (m, 2H); 4.90 (m, 1H); 6.80 (d, 1H); 6.90 (s, 1H); 7.45 (d, 1H); 7.65 (m, 2H); 7.78 (s, 1H); 8.09 (s, 1H).

MS (ESP): 464 (MH⁺) for C₂₅H₂₆FN₅O₃

The racemic mixture was separated on a Chiralpak AD, 250×20mm, 10μ column (50% methanol, 50% ethanol, 0.1% diethylamine). Example 12 eluted first, [α]_{D}=+14.3 (c=0.3, methanol) (89 mg), followed by Example 13, [α]_{D}=−11.6 (c=0.328, methanol) (80 mg).

### Example 14

### 1-(2-{(3S,4R)-4-[(2,3-Dihydro[1,4]dioxino[2,3-c]pyridin-7-ylmethyl)amino]-3-methoxypiperidin-1-yl}ethyl)-7-methoxyquinoxalin-2(1H)-one, bis-hydrochloride salt

A mixture of 1-{2-[(3S,4R)-4-amino-3-methoxypiperidin-1-yl]ethyl}-7-methoxyquinoxalin-2(1H)-one (Intermediate 31, 160 mg crude, 0.48 mmol) and 2,3-dihydro[1,4]dioxino[2,3-c]pyridine-7-carbaldehyde (80 mg, 0.48 mmol) in dry methanol/chloroform (1:1, 10 mL) was heated under nitrogen over 3 Å molecular sieves for one hour at 70° C. The reaction was allowed to cool to room temperature and sodium triacetoxy borohydride (310 mg, 1.44 mmol) was added. After 30 min, the reaction was filtered through celite. The filtrate was concentrated to dryness, taken up in 15% methanol in chloroform, and washed with saturated sodium bicarbonate solution. The aqueous phase was re-extracted twice with 15% methanol/chloroform. The combined organic phases were dried over magnesium sulfate and concentrated under reduced pressure. Chromatography on silica gel with a gradient of 2-5% methanol in dichloromethane containing 0.25% ammonium hydroxide gave 160 mg (70%) of the free base of the title composition as an oil. This was taken up in dichloromethane/diethyl ether (1:1, 5 mL) and treated with 2.0 M HCl in ether (˜2 eq). The resulting precipitate was collected by filtration, reconstituted in water and lyophilized to give 148 mg of the of the title composition as a solid.

¹H NMR (D₂O) δ: 2.04-2.33 (m, 2H); 3.05-3.25 (m, 2H); 3.44 (s, 3H); 3.50-3.71 (m, 3H); 3.87 (s, 3H); 4.04 (s, 1H); 4.21 (d, 3H); 4.26-4.32 (m, 3H); 4.33-4.40 (m, 2H); 4.45-4.58 (m, 1H); 4.74-4.87 (m, 1H); 6.82-6.92 (m, 1H); 7.02-7.12 (m, 2H); 7.75 (d, 1H); 8.01 (s, 1H); 8.09 (s, 1H).

MS (ESP): 482 (MH) for C₂₅H₃₁N₅O₅

### Example 15

### 6-[({(3S,4R)-3-Methoxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one

### 1-{2-[(3S,4R)-4-amino-3-methoxypiperidin-1-yl]ethyl}-7-methoxyquinoxalin-2(1H)-one

(Intermediate 31, 160 mg crude, 0.48 mmol), 3-oxo-3,4-dihydro-2H-pyrido[3,2-b][1,4]oxazine-6-carbaldehyde (WO 2004/058144) (85 mg, 0.48 mmol) and sodium triacetoxy borohydride (310 mg, 1.44 mmol) were reacted as described for Example 14 (except for the hydrochloride salt preparation) to give 139 mg (58%) of the title composition.

¹H NMR (DMSO-d₆) δ: 1.36-1.53 (m, 1H); 1.59-1.77 (m, 1H); 2.24-2.37 (m, 1H); 2.40-2.46 (m, 1H); 2.60 (t, 2H); 2.65-2.84 (m, 2H); 3.14-3.21 (m, 3H); 3.30-3.34 (m, 2H); 3.67 (q, 2H); 3.92 (s, 3H); 4.22-4.45 (m, 2H); 4.61 (s, 2H); 6.92-7.10 (m, 3H); 7.30 (d, 1H); 7.75 (d, 1H); 8.04 (s, 1H); 11.15-11.27 (m, 1H).

MS (ESP): 495 (MH⁺) for C₂₅H₃₀N₆O₅

### Example 16

### 1-(2-{(3R,4S)-4-[(2,3-Dihydro[1,4]dioxino[2,3-c]pyridin-7-ylmethyl)amino]-3-methoxypiperidin-1-yl}ethyl)-7-methoxyquinoxalin-2(1H)-one, bis-hydrochloride salt

1-{2-[(3R,4S)-4-Amino-3-methoxypiperidin-1-yl]ethyl}-7-methoxyquinoxalin-2(1H)-one (Intermediate 37, 37 mg, 0.11 mmol), 2,3-dihydro[1,4]dioxino[2,3-c]pyridine-7-carbaldehyde (WO 2004/058144) (18 mg, 0.11 mmol) and sodium triacetoxy borohydride (70 mg, 0.33 mmol) were reacted as described according to Example 14 to give 29 mg of the title composition as a colorless solid.

¹H NMR (D₂O) δ: 2.17-2.30 (m, 2H); 3.12-3.24 (m, 2H); 3.49 (s, 3H); 3.56-3.75 (m, 3H); 3.92 (s, 3H); 4.09 (s, 1H); 4.25 (d, 2H); 4.34 (dd, 2H); 4.41 (dd, 2H); 4.52-4.70 (m, 2H); 4.79-4.93 (m, 2H); 6.93 (d, 1H); 7.08-7.16 (m, 2H); 7.82 (d, 1H); 8.07 (s, 1H); 8.13 (s, 1H).

MS (ESP): 482 (MH⁺) for C₂₅H₃₁N₅O₅

### Example 17

### 6-[({(3R,4S)-3-Methoxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one

### 1-{2-[(3R,4S)-4-Amino-3-methoxypiperidin-1-yl]ethyl}-7-methoxyquinoxalin-2(1H)-one

(Intermediate 37, 37 mg crude, 0.11 mmol), 3-oxo-3,4-dihydro-2H-pyrido[3,2-b][1,4]oxazine-6-carbaldehyde (WO 2004/058144) (20 mg, 0.11 mmol) and sodium triacetoxy borohydride (70 mg, 0.33 mmol) were reacted as described for Example 14 (except for the hydrochloride salt preparation) to give 34 mg (63%) of the title composition.

¹H NMR (DMSO-d₆) δ: 1.39-1.53 (m, 1H); 1.59-1.77 (m, 1H); 2.29 (d, 1H); 2.38-2.47 (m, 1H); 2.60 (t, 2H); 2.65-2.85 (m, 3H); 3.13-3.22 (m, 3H); 3.30-3.34 (m, 2H); 3.58-3.78 (m, 2H); 3.92 (s, 3H); 4.24-4.44 (m, 2H); 4.61 (s, 2H); 6.93-7.07 (m, 3H); 7.30 (d, 1H); 7.75 (d, 1H); 8.04 (s, 1H); 11.20 (s, 1H).

MS (ESP): 495 (MH⁺) for C₂₅H₃₀N₆O₅

### Example 18

### 6-[({(3R,4S)-3-Methoxy-1-[2-(7-methoxy-2-oxoquinoxalin-1(2H)-yl)ethyl]piperidin-4-yl}amino)methyl]-2H-pyrido[3,2-b][1,4]thiazin-3(4H)-one, bis-hydrochloride salt

1-{2-[(3R,4S)-4-Amino-3-methoxypiperidin-1-yl]ethyl}-7-methoxyquinoxalin-2(1H)-one (Intermediate 37, 70 mg crude, 0.21 mmol), 3-oxo-3,4-dihydro-2H-pyrido[3,2-b][1,4]thiazine-6-carbaldehyde (WO 2004/058144) (40 mg, 0.21 mmol) and sodium triacetoxy borohydride (130 mg, 0.63 mmol) were reacted using a procedure similar to the one described for the synthesis of Example 14 to give 73 mg of the title composition as an off-white solid.

¹H NMR (D₂O) δ: 2.13-2.28 (m, 2H); 3.03-3.21 (m, 2H); 3.41-3.46 (m, 3H); 3.50 (s, 2H); 3.52-3.72 (m, 4H); 3.86 (s, 3H); 4.08 (s, 1H); 4.19-4.35 (m, 3H); 4.44-4.59 (m, 1H); 4.74-4.88 (m, 1H); 6.86 (d, 1H); 7.01-7.10 (m, 2H); 7.70-7.79 (m, 2H); 8.01 (s, 1H).

MS (ESP): 511 (MH⁺) for C₂₅H₃₀N₆O₄S

### Example 19

### 1-(2-{(3S,4R)-4-[(2,3-Dihydro[1,4]dioxino[2,3-c]pyridin-7-ylmethyl)amino]-3-methoxypiperidin-1-yl}ethyl)-2-oxo-1,2-dihydroquinoline-7-carbonitrile, bis-hydrochloride salt

1-{2-[(3S,4R)-4-amino-3-methoxypiperidin-1-yl]ethyl}-2-oxo-1,2-dihydroquinoline-7-carbonitrile (Intermediate 41, 93 mg, 0.29 mmol), 2,3-dihydro[1,4]dioxino[2,3-c]pyridine-7-carbaldehyde (WO 2004/058144) (47 mg, 0.29 mmol) and sodium triacetoxy borohydride (180 mg, 0.86 mmol) were reacted using a procedure similar to the one described for the synthesis of Example 14 to give 95 mg of the title composition as a colorless solid.

¹H NMR (D₂O) δ: 2.14-2.33 (m, 2H); 3.12-3.27 (m, 2H); 3.42-3.49 (m, 3H); 3.54-3.62 (m, 2H); 3.63-3.76 (m, 2H); 4.09 (s, 1H); 4.25 (d, 1H); 4.32-4.39 (m, 4H); 4.43-4.50 (m, 2H); 4.50-4.65 (m, 1H); 4.83 (d, 1H); 6.80 (d, 1H); 7.29 (s, 1H); 7.61 (dd, 1H); 7.82 (d, 1H); 7.91 (s, 1H); 7.97 (d, 1H); 8.22 (s, 1H).

MS (ESP): 476 (MH⁺) for C₂₆H₂₉N₅O₄

### Example 20

### 1-[2-((3S,4R)-3-Methoxy-4-{[(3-oxo-3,4-dihydro-2H-pyrido[3,2-][1,4]oxazin-6-yl)methyl]amino}piperidin-1-yl)ethyl]-2-oxo-1,2-dihydroquinoline-7-carbonitrile

1-{2-[(3S,4R)-4-amino-3-methoxypiperidin-1-yl]ethyl}-2-oxo-1,2-dihydroquinoline-7-carbonitrile (Intermediate 41, 93 mg, 0.29 mmol), 3-oxo-3,4-dihydro-2H-pyrido[3,2-b][1,4]oxazine-6-carbaldehyde (WO 2004/058144) (51 mg, 0.29 mmol) and sodium triacetoxy borohydride (180 mg, 0.86 mmol) were reacted using a procedure similar to the one described for the synthesis of Example 14 (except for the hydrochloride salt preparation) to give 80 mg (57%) of the title compound as an off white solid.

¹H NMR (DMSO-d₆) δ: 1.36-1.51 (m, 1H); 1.59-1.75 (m, 1H); 2.23-2.37 (m, 1H); 2.56 (t, 2H); 2.60-2.80 (m, 3H); 3.17 (s, 3H); 3.26-3.32 (m, 2H); 3.68 (q, 2H); 4.25-4.45 (m, 2H); 4.61 (s, 2H); 6.78 (d, 1H); 6.99 (d, 1H); 7.29 (d, 1H); 7.65 (dd, 1H); 7.91 (d, 1H); 8.00 (d, 1H); 8.10 (s, 1H); 11.20 (s, 1H).

MS (ESP): 489 (MH⁺) for C₂₆H₂₈N₆O₄

### Example

### 1-[2-((3S,4R)-3-Methoxy-4-{[(3-oxo-3,4-dihydro-2H-pyrido[3,2-b][1,4]thiazin-6-yl)methyl]amino}piperidin-1-yl)ethyl]-2-oxo-1,2-dihydroquinoline-7-carbonitrile, bis-hydrochloride salt

1-{2-[(3S,4R)-4-Amino-3-methoxypiperidin-1-yl]ethyl}-2-oxo-1,2-dihydroquinoline-7-carbonitrile (Intermediate 41, 93 mg, 0.29 mmol), 3-oxo-3,4-dihydro-2H-pyrido[3,2-b][1,4]thiazine-6-carbaldehyde (WO 2004/058144) (55 mg, 0.29 mmol) and sodium triacetoxy borohydride (180 mg, 0.86 mmol) were reacted using a procedure similar to the one described for the synthesis of Example 14 to give 68 mg of the title composition as a yellow solid.

¹H NMR (D₂O) δ: 2.14-2.27 (m, 2H); 3.00-3.15 (m, 2H); 3.44 (s, 3H); 3.47-3.53 (m, 4H); 3.57-3.70 (m, 2H); 4.05 (s, 1H); 4.12-4.21 (m, 1H); 4.27 (d, 2H); 4.47-4.64 (m, 1H); 4.72-4.86 (m, 1H); 6.80 (d, 1H); 7.05 (d, 1H); 7.61 (dd, 1H); 7.75 (d, 1H); 7.82 (d, 1H); 7.91 (s, 1H); 7.97 (d, 1H).

MS (ESP): 505 (MH⁺) for C₂₆H₂₈N₆O₃S

### Example 22

### 1-(2-{(3R,4S)-4-[(2,3-Dihydro[1,4]dioxino[2,3-c]pyridin-7-ylmethyl)amino]-3-methoxypiperidin-1-yl}ethyl)-2-oxo-1,2-dihydroquinoline-7-carbonitrile, bis-hydrochloride salt

1-{2-[(3R,48)-4-Amino-3-methoxypiperidin-1-yl]ethyl}-2-oxo-1,2-dihydroquinoline-7-carbonitrile (Intermediate 43, 80 mg, 0.25 mmol), 2,3-dihydro[1,4]dioxino[2,3-c]pyridine-7-carbaldehyde (WO 2004/058144) (41 mg, 0.25 mmol) and sodium triacetoxy borohydride (160 mg, 0.74 mmol) were reacted using a procedure similar to the one described for the synthesis of Example 14 to give 69 mg of the title composition as an off-white solid.

¹H NMR (D₂O) δ: 2.14-2.30 (m, 2H); 3.12-3.25 (m, 2H); 3.41-3.48 (m, 314); 3.54-3.62 (m, 2H); 3.63-3.74 (m, 2H); 4.08 (s, 1H); 4.26 (s, 1H); 4.30-4.38 (m, 4H); 4.40-4.47 (m, 2H); 4.53-4.66 (m, 1H); 4.75-4.90 (m, 1H); 6.80 (d, 1H); 7.24 (s, 1H); 7.62 (dd, 1H); 7.83 (d, 1H); 7.92 (s, 1H); 7.98 (d, 1H); 8.19 (s, 1H).

MS (ESP): 476 (MH⁺) for C₂₆H₂₉N₅O₄

### Example 23

### 1-[2-((3R,4S)-3-Methoxy-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile

1-{2-[(3R,4S)-4-Amino-3-methoxypiperidin-1-yl]ethyl}-2-oxo-1,2-dihydroquinoline-7-carbonitrile (Intermediate 43, 80 mg, 0.25 mmol), 3-oxo-3,4-dihydro-2H-pyrido[3,2-b][1,4]oxazine-6-carbaldehyde (WO 2004/058144) (45 mg, 0.25 mmol) and sodium triacetoxy borohydride (160 mg, 0.74 mmol) were reacted using a procedure similar to the one described for the synthesis of Example 14 (except for the hydrochloride salt preparation) to give 62 mg (55% yield) of the title compound as an off white solid.

¹H NMR (DMSO-d₆) δ: 1.36-1.51 (m, 1H); 1.61-1.74 (m, 1H); 2.23-2.36 (m, 1H); 2.58 (t, 2H); 2.61-2.85 (m, 3H); 3.13-3.21 (m, 3H); 3.26-3.32 (m, 2H); 3.58-3.78 (m, 2H); 4.30-4.45 (m, 2H); 4.61 (s, 2H); 6.78 (d, 1H); 7.00 (d, 1H); 7.30 (d, 1H); 7.65 (dd, 1H); 7.91 (d, 1H); 8.00 (d, 1H); 8.07-8.13 (m, 1H); 11.21 (s, 1H).

MS (ESP): 489 (MH⁺) for C₂₆H₂₈N₆O₄

### Example 24

### 1-[2-((3R,4S)-3-Methoxy-4-{[(3-oxo-3,4-dihydro-2H-pyrido[3,2-b][1,4]thiazin-6-yl)methyl]amino}piperidin-1-yl)ethyl]-2-oxo-1,2-dihydroquinoline-7-carbonitrile, bis-hydrochloride salt

1-{2-[(3R,4S)-4-Amino-3-methoxypiperidin-2-yl]ethyl}-2-oxo-1,2-dihydroquinoline-7-carbonitrile (Intermediate 43, 80 mg, 0.25 mmol), 3-oxo-3,4-dihydro-2H-pyrido[3,2-b][1,4]thiazine-6-carbaldehyde (WO 2004/058144) (48 mg, 0.25 mmol) and sodium triacetoxy borohydride (160 mg, 0.74 mmol) were reacted as described for Example 14 to give 56 mg of the title composition as an off white solid.

¹H NMR (D₂O) δ: 2.10-2.24 (m, 2H); 2.89-3.13 (m, 2H); 3.38-3.46 (m, 5H); 3.48-3.52 (m, 2H); 3.53-3.68 (m, 2H); 4.04 (s, 1H); 4.11 (d, 1H); 4.27 (d, 2H); 4.49-4.62 (m, 1H); 4.73-4.84 (m, 1H); 6.79 (d, 1H); 7.05 (d, 1H); 7.61 (dd, 1H); 7.75 (d, 1H); 7.82 (d, 1H); 7.91 (s, 1H); 7.97 (d, 1H).

MS (ESP): 505 (MH⁺) for C₂₆H₂₈N₆O₃S

### Example 25

### 1-(2-{(3S,4S)-4-[(2,3-Dihydro[1,4]dioxino[2,3-c]pyridin-7-ylmethyl)amino]-3-methoxypiperidin-1-yl}ethyl)-7-methoxyquinoxalin-2(1H)-one, bis-hydrochloride salt

1-{2-[(3S,4S)-4-Amino-3-methoxypiperidin-1-yl]ethyl}-7-methoxyquinoxalin-2(1H)-one (Intermediate 48, 70 mg, 0.21 mmol), 2,3-dihydro[1,4]dioxino[2,3-c]pyridine-7-carbaldehyde (WO 2004/058144) (35 mg, 0.21 mmol) and sodium triacetoxy borohydride (130 mg, 0.63 mmol) were reacted using a procedure similar to the one described for the synthesis of Example 14 to give 61 mg of the title composition as a yellow solid.

¹H NMR (D₂O) δ: 1.83-2.02 (m, 1H); 2.37-2.50 (m, 1H); 2.90-3.06 (m, 1H); 3.08-3.23 (m, 1H); 3.33-3.48 (m, 4H); 3.55-3.70 (m, 3H); 3.73-3.81 (m, 1H); 3.87 (s, 3H); 4.16-4.26 (m, 1H); 4.32-4.39 (m, 4H); 4.43-4.51 (m, 2H); 4.60-4.80 (m, 2H); 6.87 (d, 1H); 7.07 (dd, 1H); 7.27 (s, 1H); 7.76 (d, 1H); 8.01 (s, 1H); 8.20 (s, 1H).

MS (ESP): 482 (MH⁺) for C₂₅H₃₁N₅O₅

### Example 26

### 6-[({(3S,4S)-3-Methoxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one

1-{2-[(3S,4S)-4-Amino-3-methoxypiperidin-1-yl}ethyl -7-methoxyquinoxalin-2(1H)-one (Intermediate 48, 70 mg, 0.21 mmol), 3-oxo-3,4-dihydro-2H-pyrido[3,2-b][1,4]oxazine-6-carbaldehyde (WO 2004/058144) (37 mg, 0.21 mmol) and sodium triacetoxy borohydride (130 mg, 0.63 mmol) were reacted as described for Example 14 (except for the hydrochloride salt preparation). The crude product was subjected to flash chromatography eluting with a gradient of 0-10% methanol/dichloromethane to give 62 mg (62%) of the title compound, [α]_{D}=−19.5 (methanol, c=0.58).

¹H NMR (DMSO-d₆) δ: 1.04-1.23 (m, 1H); 1.76 (t, 1H); 1.81-1.91 (m, 1H); 1.93-2.07 (m, 1H); 2.18-2.42 (m, 1H); 2.61 (t, 2H); 2.79-2.99 (m, 2H); 3.20-3.30 (m, 4H); 3.54-3.75 (m, 2H); 3.91 (s, 3H); 4.25-4.43 (m, 2H); 4.59 (s, 2H); 6.90-7.06 (m, 3H); 7.27 (d, 1H); 7.74 (d, 1H); 8.02-8.06 (m, 1H); 11.18 (s, 1H).

MS (ESP): 495 (MH⁺) for C₂₅H₃₀N₆O₅

### Example 27

### 1-(2-{(3R,4R)-4-[(2,3-Dihydro[1,4]dioxino[2,3-c]pyridin-7-ylmethyl)amino]-3-methoxypiperidin-1-yl}ethyl)-7-methoxyquinoxalin-2(1H)-one, bis-hydrochloride salt

1-{2-[(3R,4R)-4-Amino-3-methoxypiperidin-1-yl]ethyl}-7-methoxyquinoxalin-2(1H)-one (Intermediate 56, 75 mg, 0.23 mmol), 2,3-dihydro[1,4]dioxino[2,3-c]pyridine-7-carbaldehyde (WO 2004/058144) (37 mg, 0.23 mmol) and sodium triacetoxy borohydride (150 mg, 0.69 mmol) were reacted as described for Example 14 to give the title composition (63 mg) as a yellow solid.

¹H NMR (D₂O) δ: 1.81-2.02 (m, 1H); 2.34-2.52 (m, 1H); 2.92-3.05 (m, 1H); 3.10-3.23 (m, 1H); 3.37-3.49 (m, 4H); 3.54-3.72 (m, 3H); 3.73-3.82 (m, 1H); 3.85-3.90 (m, 3H); 4.22 (d, 1H); 4.29-4.41 (m, 4H); 4.43-4.51 (m, 2H); 4.60-4.80 (m, 2H); 6.83-6.91 (m, 1H); 7.07 (dd, 1H); 7.26-7.31 (m, 1H); 7.74 (d, 1H); 7.97-8.03 (m, 1H); 8.17-8.26 (m, 1H).

MS (ESP): 482 (MH⁺) for C₂₅H₃₁N₅O₅

### Example 28

### 6-[({(3R,4R)-3-Methoxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one

1-{2-[(3R,4R)-4-Amino-3-methoxypiperidin-1-yl]ethyl}-7-methoxyquinoxalin-2(1H)-one (Intermediate 56, 75 mg, 0.23 mmol), 3-oxo-3,4-dihydro-2H-pyrido[3,2-b][1,4]oxazine-6-carbaldehyde (WO 2004/058144) (40 mg, 0.23 mmol) and sodium triacetoxy borohydride (150 mg, 0.69 mmol) were reacted as described for Example 14 (except for the hydrochloride salt preparation) to give 73 mg (66%) of the title compound, [α]_{D}=+17.5 (methanol, c=0.56).

¹H NMR (DMSO-d₆) δ: 1.04-1.22 (m, 1H); 1.78 (t, 1H); 1.83-1.93 (m, 1H); 2.00 (m, 1H); 2.22-2.35 (m, 1H); 2.62 (t, 2H); 2.81-3.00 (m, 2H); 3.20-3.30 (m, 4H); 3.56-3.78 (m, 2H); 3.92 (s, 3H); 4.26-4.44 (m, 2H); 4.61 (s, 2H); 6.92-7.08 (m, 3H); 7.29 (d, 1H); 7.75 (d, 1H); 8.05 (s, 1H); 11.21 (s, 1H).

MS (ESP): 495 (MH⁺) for C₂₅H₃₀N₆O₅

### Example 29

### 1-(2-{4-[(2,3-Dihydro[1,4]dioxino[2,3-c]pyridin-7-ylmethyl)amino]-3-hydroxypiperidin-1-yl}ethyl)-7-methoxyquinoxalin-2(1H)-one, trans enantiomer A, bis hydrochloride salt

1-{2-[4-Amino-3-hydroxypiperidin-1-yl]ethyl}-7-methoxyquinoxalin-2(1H)-one, trans enantiomer A (Intermediate 57, 39 mg, 0.12 mmol), 2,3-dihydro[1,4]dioxino[2,3-c]pyridine-7-carbaldehyde (WO 2004/058144) (20 mg, 0.12 mmol) and sodium triacetoxy borohydride (76 mg, 0.36 mmol) were reacted as described for Example 14 to give the title composition as a yellow solid (39 mg).

¹H NMR (D₂O) δ: 1.82-2.00 (m, 1H); 2.48 (dd, 1H); 3.01 (t, 1H); 3.08-3.21 (m, 1H); 3.30-3.44 (m, 1H); 3.58 (t, 2H); 3.79-3.95 (m, 5H); 3.97-4.09 (m, 1H); 4.30-4.50 (m, 6H); 4.65 (t, 2H); 6.87 (d, 1H); 7.07 (dd, 1H); 7.28 (s, 1H); 7.76 (d, 1H); 8.01 (s, 1H); 8.20 (s, 1H).

MS (ESP): 468 (MH⁺) for C₂₄H₂₉N₅O₅

### Example 30

### 6-[({3-Hydroxy-1-[2-(7-methoxy-2-oxoquinoxalin-1(2H)-yl)ethyl]piperidin-4-yl}amino)methyl]-2H-pyrido[3,2-b][1,4]oxazin-3(4H-one, trans enantiomer A

1-{2-[4-amino-3-hydroxypiperidin-1-yl]ethyl}-7-methoxyquinoxalin-2(1H)-one, trans enantiomer A (Intermediate 57, 39 mg, 0.12 mmol), 3-oxo-3,4-dihydro-2H-pyrido[3,2-b][1,4]oxazine-6-carbaldehyde (WO 2004/058144) (21 mg, 0.12 mmol) and sodium triacetoxy borohydride (76 mg, 0.36 mmol) were reacted as described for Example 14 (except for the hydrochloride salt preparation) to give 49 mg (84%) of the title compound.

¹H NMR (DMSO-d₆) δ: 1.09-1.26 (m, 1H); 1.94 (t, 2H); 2.04 (t, 1H); 2.20-2.35 (m, 1H); 2.64 (t, 2H); 2.88-3.10 (m, 2H); 3.18-3.32 (m, 1H); 3.63-3.87 (m, 2H); 3.98 (s, 3H); 4.39 (t, 2H); 4.67 (s, 2H); 4.90 (d, 1H); 6.99-7.11 (m, 3H); 7.36 (d, 1H); 7.81 (d, 1H); 8.10 (s, 1H); 11.27 (s, 1H).

MS (ESP): 481 (MH⁺) for C₂₄H₂₈N₆O₅

### Example 31

### 1-(2-{4-[(2,3-dihydro[1,4]dioxino[2,3-c]pyridin-7-ylmethyl)amino]-3-hydroxypiperidin-1-yl}ethyl)-7-methoxyquinoxalin-2(1H)-one, trans enantiomer B, bis-hydrochloride salt

1-{2-[4-amino-3-hydroxypiperidin-1-yl]ethyl}-7-methoxyquinoxalin-2(1H)-one, trans enantiomer (Intermediate 65, 42 mg, 0.13 mmol), 2,3-dihydro[1,4]dioxino[2,3-c]pyridine-7-carbaldehyde (WO 2004/058144) (22 mg, 0.13 mmol) and sodium triacetoxy borohydride (83 mg, 0.39 mmol) were reacted as described for Example 14 to give 45 mg of the title composition as a yellow solid.

¹H NMR (D₂O) δ: 1.82-1.99 (m, 1H); 2.42-2.53 (m, 1H); 3.01 (t, 1H); 3.08-3.20 (m, 1H); 3.32-3.44 (m, 1H); 3.58 (t, 2H); 3.80-3.95 (m, 5H); 3.96-4.09 (m, 1H); 4.32-4.50 (m, 6H); 4.65 (t, 2H); 6.87 (d, 1H); 7.07 (dd, 1H); 7.29 (s, 1H); 7.76 (d, 1H); 8.00 (s, 1H); 8.21 (s, 1H).

MS (ESP): 467 (MH⁺) for C₂₄H₂₉N₅O₅

### Example 32

### 6-[({3-Hydroxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one, trans enantiomer B

1-{2-[4-Amino-3-hydroxypiperidin-1-yl]ethyl}-7-methoxyquinoxalin-2(1H)-one, trans enantiomer (Intermediate 65, 42 mg, 0.13 mmol), 3-oxo-3,4-dihydro-2H-pyrido[3,2-b][1,4]oxazine-6-carbaldehyde (WO 2004/058144) (24 mg, 0.13 mmol) and sodium triacetoxy borohydride (83 mg, 0.39 mmol) were reacted as described for Example 14 (except for the hydrochloride salt preparation) to give 42 mg (68% yield) of the title compound.

¹H NMR (DMSO-d₆) δ: 1.02-1.21 (m, 1H); 1.88 (t, 2H); 1.98 (t, 1H); 2.15-2.31 (m, 1H); 2.59 (t, 2H); 2.84-3.03 (m, 2H); 3.13-3.25 (m, 1H); 3.58-3.80 (m, 2H); 3.92 (s, 3H); 4.32 (t, 2H); 4.61 (s, 2H); 4.84 (d, 1H); 6.94-7.05 (m, 3H); 7.30 (d, 1H); 7.68-7.79 (m, 1H); 8.04 (s, 1H); 11.16-11.29 (m, 1H).

MS (ESP): 481 (MH⁺) for C₂₄H₂₈N₆O₅

### Example 33

### 1-(2-{4-[(2,3-Dihydro[1,4]dioxino[2,3-c]pyridin-7-ylmethyl)amino]-3-fluoroniperidin-1-yl}ethyl)-2-oxo-1,2-dihydroquinoline-7-carbonitrile, trans enantiomer A, bis-hydrochloride salt

A mixture of 1-{2-[4-amino-3-fluoropiperidin-1-yl]ethyl}-2-oxo-1,2-dihydroquinoline-7-carbonitrile, trans enantiomer A (Intermediate 67, crude, 150 mg, 0.48 mmol) and 2,3-dihydro[1,4]dioxino[2,3-c]pyridine-7-carbaldehyde (WO 2004/058144) (80 mg, 0.48 mmol) in dry methanol/chloroform (1:1, 20 mL) was heated under nitrogen over 3 Å molecular sieves for one hour at 70° C. The reaction was allowed to cool to room temperature and sodium triacetoxy borohydride (300 mg, 1.43 mmol) was added. The reaction was stirred at room temperature overnight. The reaction mixture was filtered through celite. The filtrate was concentrated to dryness. The crude product was taken up in 15% methanol/chloroform and washed with saturated sodium bicarbonate solution. The aqueous phase was re-extracted once with 15% methanol/chloroform. The combined organic phases were dried over sodium sulfate, filtered, and concentrated under reduced pressure. Chromatography on silica gel with a gradient of 0-5% methanol in dichloromethane gave 66 mg (26%) of the free base of the title composition as an oil. This was taken up in 1:1 dichloromethane/diethyl ether (5 mL) and treated with 1.0 M HCl in ether (˜2 eq) resulting in a precipitate. This mixture was concentrated to dryness. The resulting solid was reconstituted in water and lyophilized to give 66 mg of the title composition.

¹H NMR (D₂O) δ ppm: 1.81-1.94 (m, 1H); 2.30-2.43 (m, 1H); 3.13-3.26 (m, 1H); 3.32-3.42 (m, 1H); 3.42-3.50 (m, 1H); 3.53 (t, 2H); 3.57-3.66 (m, 1H); 3.79-3.93 (m, 1H); 4.11-4.27 (m, 2H); 4.38-4.43 (m, 2H); 4.50-4.56 (m, 2H); 4.62-4.75 (m, 1H); 4.80-4.88 (m, 1H); 4.85 (m, 1H) 6.85 (d, 1H); 7.25 (s, 1H); 7.68 (dd, 1H); 7.89 (d, 1H); 7.98 (s, 1H); 8.03 (d, 1H); 8.20 (s, 1H).

MS (ESP): 464 (MH⁺) for C₂₅H₂₆FN₅O₃

### Example 34

### 1-[2-((3-Fluoro-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile, trans enantiomer A

1-{2-[4-Amino-3-fluoropiperidin-1-yl]ethyl}-2-oxo-1,2-dihydroquinoline-7-carbonitrile, trans enantiomer A (Intermediate 67, 150 mg, 0.48 mmol), 3-oxo-3,4-dihydro-2H-pyrido[3,2-b][1,4]oxazine-6-carbaldehyde (WO 2004/058144) (85 mg, 0.48 mmol), and sodium triacetoxyborohydride (300 mg, 1.43 mmol) were reacted as described for Example 33 (except for the hydrochloride salt preparation) to give 70 mg (30%) the title composition. [α]_{D}=−12.4 (methanol, c=0.5).

¹H NMR (DMSO-d₆) δ ppm: 1.21 (m, 1H); 1.84-1.95 (m, 1H); 2.04-2.21 (m, 2H); 2.30-2.41 (m, 1H); 2.57-2.67 (m, 2H); 2.78-2.87 (m, 1H); 3.18-3.29 (m, 1H); 3.73 (s, 2H); 4.29 (m, 1H); 4.29-4.46 (m, 2H); 4.61 (s, 2H); 6.78 (d, 1H); 7.01 (d, 1H); 7.30 (d, 1H); 7.66 (dd, 1H); 7.91 (d, 1H); 8.01 (d, 1H); 8.11 (s, 1H); 11.19 (s, 1H).

MS (ESP): 477 (MH⁺) for C₂₅H₂₅FN₆O₃

Examples 35 to 40 were purified by reverse phase HPLC on a Waters XBridge C-18 column eluting with 5-50% water/acetonitrile with ammonium acetate buffer (pH=8).

### Example 35

### 1-(2-{4-[(2,3-Dihydro[1,4]dioxino[2,3-c]pyridin-7-ylmethyl)amino]-3-fluoropiperidin-1-yl}ethyl)-7-methoxyquinoxalin-2(1H)-one, trans enantiomer A, bis hydrochloride salt

2,3-Dihydro[1,4]dioxino[2,3-c]pyridine-7-carbaldehyde and 1-{2-[4-Amino-3-fluoropiperidin-1-yl]ethyl}-7-methoxyquinoxalin-2(1H)-one, trans enantiomer A (Intermediate 77) were reacted using a procedure similar to the one described for the synthesis of Example 33, providing the title composition.

¹H NMR (D₂O) δ ppm: 1.81-1.97 (m, 1H); 2.32-2.45 (m, 1H); 3.20-3.32 (m, 1H); 3.35-3.44 (m, 1H); 3.52-3.72 (m, 4H); 3.91 (s, 3H); 3.85-3.95 (m, 1H); 4.13-4.28 (m, 2H); 4.37-4.43 (m, 2H); 4.51-4.56 (m, 2H); 4.57-4.73 (m, 2H); 4.94 (m, 1H); 6.90 (d, 1H); 7.10 (dd, 1H); 7.30 (s, 1H); 7.78 (d, 1H); 8.03 (s, 1H); 8.22 (s, 1H).

ES (MH)⁺470

### Example 36

### 6-[({3-Fluoro-1-[2-(7-methoxy-2-oxoquinoxalin-1(2H)-yl)ethyl]piperidin-4-yl}amino)methyl]-2H-pyrido[3,2-b][1,4]oxazin-3(4H)-one, trans enantiomer A

3-Oxo-3,4-dihydro-2H-pyrido[3,2-b][1,4]oxazine-6-carbaldehyde and 1-{2-[4-Amino-3-fluoropiperidin-1-yl]ethyl}-7-methoxyquinoxalin-2(1H)-one, trans enantiomer A (Intermediate 77) were reacted using a procedure similar to the one described for the synthesis of Example 33 (except for the hydrochloride salt preparation), providing the title compound.

¹H NMR (DMSO-d₆) δ ppm: 1.14-1.29 (m, 1H); 1.82-1.95 (m, 1H); 2.04-2.20 (m, 2H); 2.52-2.61 (m, 1H); 2.60-2.70 (m, 2H); 2.78-2.89 (m, 1H); 3.16-3.27 (m, 1H); 3.67-3.80 (m, 2H); 3.88-3.96 (m, 3H); 4.29-4.43 (m, 2H); 4.30 (m, 1H); 4.61 (s, 2H); 6.96-7.06 (m, 3H); 7.30 (d, 1H); 7.75 (d, 1H); 8.00-8.09 (m, 1H); 11.19 (s, 1H).

ES (MH)⁺483

### Example 37

### 1-(2-{4-[(2,3-Dihydro[1,4]dioxino[2,3-c]pyridin-7-ylmethyl)amino]-3-fluoropiperidin-1-yl}ethyl)-2-oxo-1,2-dihydroquinoline-7-carbonitrile, trans enantiomer B, bis-hydrochloride salt

2,3-dihydro[1,4]dioxino[2,3-c]pyridine-7-carbaldehyde and 1-{2-[4-Amino-3-fluoropiperidin-1-yl]ethyl}-2-oxo-1,2-dihydroquinoline-7-carbonitrile, trans enantiomer B (Intermediate 79) were reacted using a procedure similar to the one described for the synthesis of Example 33, providing the title composition.

¹H NMR (D₂O) δ ppm: 1.81-1.94 (m, 1H); 2.30-2.43 (m, 1H); 3.13-3.26 (m, 1H); 3.32-3.42 (m, 1H); 3.42-3.50 (m, 1H); 3.53 (t, 2H); 3.57-3.66 (m, 1H); 3.79-3.93 (m, 1H); 4.11-4.27 (m, 2H); 4.38-4.43 (m, 2H); 4.50-4.56 (m, 2H); 4.62-4.75 (m, 1H); 4.80-4.88 (m, 1H); 4.85 (m, 1H) 6.85 (d, 1H); 7.25 (s, 1H); 7.68 (dd, 1H); 7.89 (d, 1H); 7.98 (s, 1H); 8.03 (d, 1H); 8.20 (s, 1H)

ES (MH)⁺464

### Example

### 1-[2-(3-Fluoro-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile, trans enantiomer B

3-Oxo-3,4-dihydro-2H-pyrido[3,2-b][1,4]oxazine-6-carbaldehyde and 1-{2-[4-Amino-3-fluoropiperidin-1-yl]ethyl}-2-oxo-1,2-dihydroquinoline-7-carbonitrile, trans enantiomer B (Intermediate 79) were reacted using a procedure similar to the one described for the synthesis of Example 33 (except for the hydrochloride salt preparation), providing the title compound.

¹H NMR (DMSO-d₆) δ ppm: 1.21 (m, 1H) 1.84-1.95 (m, 1H); 2.04-2.21 (m, 2H); 2.30-2.41 (m, 1H); 2.57-2.67 (m, 2H); 2.78-2.87 (m, 1H); 3.18-3.29 (m, 1H); 3.73 (s, 2H); 4.29 (m, 1H) 4.29-4.46 (m, 2H); 4.61 (s, 2H); 6.78 (d, 1H); 7.01 (d, 1H); 7.30 (d, 1H); 7.66 (dd, 1H); 7.91 (d, 1H); 8.01 (d, 1H); 8.11 (s, 1H); 11.19 (s, 1H)

ES (MH)⁺477 [α]_{D}=+19.8 (methanol, c=0.46)

### Example 39

### 1-(2-{4-[(2,3-Dihydro[1,4]dioxino[2,3-c]pyridin-7-ylmethyl)amino]-3-fluoropiperidin-1-yl}ethyl)-7-methoxyquinoxalin-2(1H)-one, trans enatiomer B, bis-hydrochloride salt

2,3-Dihydro[1,4]dioxino[2,3-c]pyridine-7-carbaldehyde and 1-{2-[4-Amino-3-fluoropiperidin-1-yl]ethyl}-7-methoxyquinoxalin-2(1H)-one, trans enantiomer B (Intermediate 83) were reacted using a procedure similar to the one described for the synthesis of Example 33, providing the title composition.

¹H NMR (D₂O) δ ppm: 1.81-1.97 (m, 1H); 2.32-2.45 (m, 1H); 3.20-3.32 (m, 1H); 3.35-3.44 (m, 1H); 3.52-3.72 (m, 4H); 3.91 (s, 3H); 3.85-3.95 (m, 1H); 4.13-4.28 (m, 2H); 4.37-4.43 (m, 2H); 4.51-4.56 (m, 2H); 4.57-4.73 (m, 2H); 4.94 (m, 1H); 6.90 (d, 1H); 7.30 (s, 1H); 7.78 (d, 1H); 8.03 (s, 1H); 8.22 (s, 1H).

ES (MH)⁺470

### Example 40

### 6-[({3-Fluoro-1-[2-(7-methoxy-2-oxoquinoxalin-1(2H)-yl)ethyl]piperidin-4-yl}amino)methyl]-2H-pyrido[3,2-b][1,4]oxazin-3(4H)-one, trans enantiomer B

3-Oxo-3,4-dihydro-2H-pyrido[3,2-b][1,4]oxazine-6-carbaldehyde and 1-{2-[4-Amino-3-fluoropiperidin-1-yl]ethyl}-7-methoxyquinoxalin-2(1H)-one, trans enantiomer B (Intermediate 83) were reacted using a procedure similar to the one described for the synthesis of Example 33 (except for the hydrochloride salt preparation), providing the title compound.

¹H NMR (DMSO-d₆) δ ppm: 1.14-1.29 (m, 1H); 1.82-1.95 (m, 1H); 2.04-2.20 (m, 2H); 2.52-2.61 (m, 1H); 2.60-2.70 (m, 2H); 2.78-2.89 (m, 1H); 3.16-3.27 (m, 1H); 3.67-3.80 (m, 2H); 3.88-3.96 (m, 3H); 4.29-4.43 (m, 2H); 4.30 (m, 1H); 4.61 (s, 2H); 6.96-7.30 (d, 1H); 7.75 (d, 1H); 8.00-8.09 (m, 1H); 11.19 (s, 1H)

ES (MH)⁺483

### Example 41

### 1-(2-{(3R,4R)-4-[(2,3-dihydro[1,4]dioxino[2,3-c]pyridin-7-ylmethyl)amino]-3-methoxypiperidin-1-yl}ethyl)-2-oxo-1,2-dihydroquinoline-7-carbonitrile, bis hydrochloride salt

A mixture of 1-{2-[(3R,4R)-4-amino-3-methoxypiperidin-1-yl]ethyl}-2-oxo-1,2-dihydroquinoline-7-carbonitrile (Intermediate 85, 105 mg crude, 0.32 mmol) and 2,3-dihydro[1,4]dioxino[2,3-c]pyridine-7-carbaldehyde (WO 2004/058144) (53 mg, 0.32 mmol) in dry methanol/chloroform (1:1, 10 mL) was heated under a nitrogen atmosphere over 3 Å molecular sieves for three hours at 70° C. The reaction was allowed to cool to room temperature and sodium triacetoxy borohydride (205 mg, 0.97 mmol) was added. The reaction was stirred at room temperature overnight, and then filtered through celite. The filtrate was concentrated to dryness, taken up in 15% methanol in chloroform, and washed with saturated sodium bicarbonate solution. The aqueous phase was re-extracted twice with 15% methanol/chloroform. The combined organic phases were dried over magnesium sulfate and concentrated under reduced pressure. Chromatography on silica gel with a gradient of 2-10% methanol in dichloromethane containing 0.25% ammonium hydroxide gave 61 mg (40%) of the free base of the title composition as an oil. This was taken up in 1:1 dichloromethane/diethyl ether (5 mL) and treated with 1.0 M HCl in ether (˜2 eq). The resulting precipitate was collected by filtration, reconstituted in water and lyophilized to give 63 mg of the title composition as a solid.

MS (ESP): 476 (MH⁺) for C₂₆H₂₉N₅O₄

¹H NMR (D₂O) δ ppm: 1.84-2.00 (m, 1H); 2.37-2.49 (m, 1H); 2.93-3.06 (m, 1H); 3.10-3.23 (m, 1H); 3.33-3.44 (m, 4H); 3.53-3.80 (m, 4H); 4.19 (d, 1H); 4.27-4.40 (m, 4H); 4.40-4.48 (m, 2H); 4.62-4.76 (m, 2H); 6.79 (d, 1H); 7.22 (s, 1H); 7.62 (dd, 1H); 7.83 (d, 1H); 7.91 (s, 1H); 7.97 (d, 1H); 8.14-8.20 (m, 1H).

### Example 42

### 1-[2-((3R,4R)-3-methoxy-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile

1-{2-[(3R,4R)-4-Amino-3-methoxypiperidin-1-yl]ethyl}-2-oxo-1,2-dihydroquinoline-7-carbonitrile (Intermediate 85, 105 mg, 0.32 mmol), 3-oxo-3,4-dihydro-2H-pyrido[3,2-b][1,4]oxazine-6-carbaldehyde (WO 2004/058144) (57 mg, 0.32 mmol) and sodium triacetoxy borohydride (205 mg, 0.97 mmol) were reacted using a procedure similar to the one described for the synthesis of Example 41 (except for the hydrochloride salt preparation). Chromatography on silica gel with a gradient of 2-10% methanol in dichloromethane containing 0.25% ammonium hydroxide gave 88 mg (56%) of the title compound as an off-white solid.

MS (ESP): 489 (MH⁺) for C₂₆H₂₈N₆O₄

¹H NMR (DMSO-d₆) δ ppm: 1.00-1.15 (m, 1H); 1.76 (m, 1H); 1.87 (d, 1H); 1.99 (m, 1H); 2.22-2.34 (m, 1H); 2.59 (m, 2H); 2.78-2.96 (m, 2H); 3.25-3.33 (m, 2H); 3.30 (s, 3H); 3.55-3.77 (m, 2H); 4.30-4.50 (m, 2H); 4.61 (s, 2H); 6.79 (d, 1H); 6.97 (d, 1H); 7.28 (d, 1H); 7.66 (dd, 1H); 7.91 (d, 1H); 8.01 (d, 1H); 8.12 (s, 1H); 11.21 (s, 1H).

### Example 43

### 1-(2-{(3S,4S)-4-[(2,3-Dihydro[1,4]dioxino[2,3-c]pyridin-7-ylmethynamino]-3-methoxypiperidin-1-yl}ethyl)-2-oxo-1,2-dihydroquinoline-7-carbonitrile, bis hydrochloride salt

1-{2-[(3S,45)-4-Amino-3-methoxypiperidin-1-yl]ethyl}-2-oxo-1,2-dihydroquinoline-7-carbonitrile (Intermediate 90, 120 mg, 0.37 mmol), 2,3-dihydro[1,4]dioxino[2,3-c]pyridine-7-carbaldehyde (WO 2004/058144) (61 mg, 0.37 mmol) and sodium triacetoxy borohydride (234 mg, 1.10 mmol) were reacted using a procedure similar to the one described for the synthesis of Example 41, providing the title composition (54 mg).

MS (ESP): 476 (MH⁺) for C₂₆H₂₉N₅O₄

¹H NMR (D₂O) δ ppm 1.84-2.00 (m, 1H); 2.37-2.49 (m, 1H); 2.93-3.06 (m, 1H); 3.10-3.23 (m, 1H); 3.33-3.44 (m, 4H); 3.53-3.80 (m, 4H); 4.19 (d, 1H); 4.27-4.40 (m, 4H); 4.40-4.48 (m, 2H); 4.62-4.76 (m, 2H); 6.79 (d, 1H); 7.22 (s, 1H); 7.62 (dd, 1H); 7.83 (d, 1H); 7.91 (s, 1H); 7.97 (d, 1H); 8.14-8.20 (m, 1H).

### Example 44

### 1-[2-((3S,4S)-3-Methoxy-4-{[3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile

1-{2-[(3S,4S)-4-Amino-3-methoxypiperidin-1-yl]ethyl}-2-oxo-1,2-dihydroquinoline-7-carbonitrile (Intermediate 90, 120 mg, 0.37 mmol), 3-oxo-3,4-dihydro-2H-pyrido[3,2-b][1,4]oxazine-6-carbaldehyde (WO 2004/058144) (61 mg, 0.37 mmol) and sodium triacetoxy borohydride (234 mg, 1.10 mmol) were reacted using a procedure similar to the one described for the synthesis of Example 41 (except for the hydrochloride salt preparation), providing the title compound.

MS (ESP): 489 (MH⁺) for C₂₆H₂₈N₆O₄

1H NMR (DMSO-d₆) δ ppm 1.00-1.15 (m, 1H); 1.76 (m, 1H); 1.87 (d, 1H); 1.99 (m, 1H); 2.22-2.34 (m, 1H); 2.59 (m, 2H); 2.78-2.96 (m, 2H); 3.25-3.33 (m, 2H); 3.30 (s, 3H); 3.55-3.77 (m, 2H); 4.30-4.50 (m, 2H); 4.61 (s, 2H); 6.79 (d, 1H); 6.97 (d, 1H); 7.28 (d, 1H); 7.66 (dd, 1H); 7.91 (d, 1H); 8.01 (d, 1H); 8.12 (s, 1H); 11.21 (s, 1H).

### Example 45

### 6-[({(3S,4R)-3-Fluoro-1-[2-(7-methoxy-2-oxoquinoxalin-1(2H)-yl)ethyl]piperidin-4-yl}amino)methyl]-2H-pyrido[3,2-b][1,4]oxazin-3(4H)-one, bis hydrochloride salt

and

### Example 46

### 6-[({(3R,4S)-3-Fluoro-1-[2-(7-methoxy-2-oxoquinoxalin-1(2H)-yl)ethyl]piperidin-4-yl}amino)methyl]-2H-pyrido[3,2-b[]1,4]oxazin-3(4H)-one, bis hydrochloride salt

A solution of cis(±) 1-[2-(4-amino-3-fluoropiperidin-1-yl)ethyl]-7-methoxyquinoxalin-2(1H)-one, trifluoroacetic acid salt (Intermediate 91, ˜0.53 mmol) in dichloroethane/methanol (1:1, 20 mL) was neutralized with N,N-diisopropylethylamine. 3-oxo-3,4-dihydro-2H-pyrido[3,2-][1,4]oxazine-6-carbaldehyde (WO 2004/058144) (113 mg, 0.63 mmol) was added and the reaction was stirred at reflux over 3 Å molecular sieves overnight. The reaction mixture was cooled to 0° C., and sodium cyanoborohydride (40 mg, 0.63 mmol) was added. The reaction mixture was stirred at room temperature for 2 hours, then filtered through a flitted funnel and concentrated. The residue was taken up in ethyl acetate and washed with saturated sodium bicarbonate followed by saturated sodium chloride. The organic extracts were dried over magnesium sulfate and concentrated. Reverse phase chromatography with water/methanol/trifluoroacetic acid yielded the product as trifluoroacetic acid salt. The salt was dissolved in water and chloroform and basified with saturated sodium carbonate. The layers were separated and the aqueous was extracted with chloroform. The organic extracts were dried over magnesium sulfate and concentrated to give a racemic mixture of the free bases of the title compositions as a solid, 26 mg (10%).

MS (ESP): 483 (MH⁺) for C₂₄H₂₇FN₆O₄

¹H-NMR (CDCl₃-d) δ: 1.70 (m, 4H); 2.35 (m, 2H); 2.64 (m, 1H); 2.73 (m, 2H); 3.04 (m, 1H); 3.31 (m, 1H); 3.86 (s, 2H); 3.92 (s, 3H); 4.35 (m, 2H); 4.63 (s, 2H); 4.83 (m, 1H); 6.86 (m, 1H); 6.92 (m, 1H); 6.98 (m, 1H); 7.21 (m, 1H); 7.77 (m, 1H); 8.11 (s, 1H).

The racemic mixture was separated by chiral chromatography (HPLC, Chiralcel OJ, 250×20 mm, 10μ mobile phase: 50% hexane, 25% ethanol, 25% methanol, 0.1% diethylamine). The free base of Example 45 eluted first, followed by the free base of Example 46. The hydrochloride salts were prepared by dissolving the free bases in dichloromethane (2 mL) and adding 2.2 equivalents of 4N HCl in dioxane. The resulting colorless precipitates were collected by filtration to yield 48 mg and 46 mg of Example 45 and 46, respectively.

### Example 47

### 1-[2-((3R,4S)-3-Methoxy-4-{[(3-oxo-3,4-dihydro-2H-pyrido[3,2-b][1,4]oxazin-6-yl)methyl]amino}piperidin-1-yl)ethyl]-4-methyl-2-oxo-1,2-dihydroquinoline-7-carbonitrile, bis-hydrochloride salt

1-{2-[(3R,4S)-4-Amino-3-methoxypiperidin-1-yl]ethyl}-4-methyl-2-oxo-1,2-dihydroquinoline-7-carbonitrile (Intermediate 93, ˜0.81 mmol) was suspended in chloroform/methanol (1:1, 20 mL) and N,N-diisopropylethylamine was added dropwise until solid went into solution. 3-Oxo-3,4-dihydro-2H-pyrido[3,2-b][1,4]oxazine-6-carbaldehyde (WO 2004/058144) (210 mg, 1.2 mmol) was added and the reaction was refluxed over 3 Å molecular sieves for 5 hours. Sodium triacetoxyborohydride (340 mg, 1.6 mmol) was added at 0° C. The reaction mixture was stirred at room temperature for 2 hours then filtered through a 0.45 μm membrane and concentrated. The residue was taken up in dichloromethane and saturated sodium bicarbonate. The pH of the aqueous phase was adjusted to pH˜10 with 1M sodium hydroxide solution. The aqueous phase was back extracted with dichloromethane and the combined organic phases were dried over sodium sulfate and concentrated. Reverse phase chromatography with water/acetonitrile/ammonium acetate afforded the free base of the title composition as a tan foam, 144 mg (36%). The bis-hydrochloride salt was prepared using a procedure similar to the one described for the synthesis of Example 45.

MS (ESP): 503 (MH⁺) for C₂₇H₃₀N₆O₄

¹H-NMR (DMSO-d₆) δ: 1.41 (m, 1H); 1.75 (m, 2H); 2.28 (m, 1H); 2.44 (s, 3H); 2.53 (m, 2H); 2.70 (m, 2H); 3.16 (s, 3H); 3.31 (m, 2H); 3.64 (m, 2H); 4.35 (m, 2H); 4.59 (s, 2H); 6.68 (s, 1H); 6.97 (d, 1H); 7.27 (d, 1H); 7.66 (d, 1H); 7.92 (d, 1H); 8.08 (s, 1H); 8.31 (s, 1H); 11.19 (s, 1H).

### Example 48

### 1-[2-((3S,4R)-3-Fluoro-4-{[(3-oxo-3,4-dihydro-2H-pyrido[3,2-b][1,4]oxazin-6-yl)methyl]amino}piperidin-1-yl)ethyl]-4-methyl-2-oxo-1,2-dihydroquinoline-7-carbonitrile, bis hydrochloride salt

and

### Example 49

### 1-[2-((3R,4S)-3-Fluoro-4-{[(3-oxo-3,4-dihydro-2H-pyrido[3,2-b][1,4]oxazin-6-yl)methyl]amino}piperidin-1-yl)ethyl]-4-methyl-2-oxo-1,2-dihydroquinoline-7-carbonitrile, bis hydrochloride salt, bis hydrochloride salt

Cis (±)1-[2-(4-amino-3-fluoropiperidin-1-yl)ethyl]-4-methyl-2-oxo-1,2-dihydroquinoline-7-carbonitrile (Intermediate 95, 200 mg, 0.63 mmol), 3-oxo-3,4-dihydro-2H-pyrido[3,2-b][1,4]oxazine-6-carbaldehyde (WO 2004/058144) (170 mg, 0.94 mmol) and sodium triacetoxyborohydride (260 mg, 1.26 mmol) were reacted using a procedure similar to the one described for the synthesis of Example 47. Reverse phase chromatography with water/acetonitrile/TFA provided a racemic mixture of the title compounds as a dry film, 80 mg.

The racemic mixture was separated by chiral chromatography (SFC, Chiralpak AD-H, 250×21 mm, 5μ; 50% methanol, 0.1% dimethylethylamine. The free base of Example 48 eluted first, followed by the free base of Example 49. The bis-hydrochloride salts were prepared using a procedure similar to the one described for the synthesis of Example 45, providing the title compounds as colorless solids (6 mg and 4 mg, respectively).

MS (ESP): 491 (MH⁺) for C₂₆H₂₇FN₆O₃

¹H-NMR DMSO-d₆) δ: 1.60 (m, 2H); 2.12 (m, 4H); 2.44 (s, 3H); 2.54 (m, 2H); 2.91 (m, 1H); 3.12 (m, 1H); 3.71 (m, 2H); 4.34 (m, 2H); 4.59 (s, 2H); 4.70 (m, 1H); 6.68 (s, 1H); 7.01 (d, 1H); 7.28 (d, 1H); 7.66 (m, 1H); 7.92 (d, 1H); 8.07 (s, 1H); 11.18 (s, 1H).

### Example 50

### 1-[2-((3S,4R)-3-Fluoro-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile, bis hydrochloride salt

1-{2-[(3S,4R)-4-amino-3-fluoropiperidin-1-yl]ethyl}-2-oxo-1,2-dihydroquinolie-7-carbonitrile trifluoroacetate (Intermediate 97, 1.2 mmol) was suspended in chloroform/methanol (1:2, 30 mL) and neutralized by dropwise addition of N,N-diisopropylethylamine. 3-Oxo-3,4-dihydro-2H-pyrido[3,2-b][1,4]oxazine-6-carbaldehyde (WO 2004/058144) (258 mg, 1.45 mmol) was added and the reaction was refluxed over 3 Å molecular sieves for 5 hours. Sodium triacetoxyborohydride (512 mg, 2.42 mmol) was added at 0° C. The reaction mixture was stirred at room temperature for 30 minutes then filtered through a fitted funnel and concentrated. The residue was taken up in dichloromethane and washed with saturated sodium bicarbonate and saturated sodium chloride. The organic phase was dried over magnesium sulfate and concentrated. Reverse phase chromatography with water/acetonitrile/ammonium acetate afforded a product as an off-white solid after lyophilization. The hydrochloride salt was prepared by dissolving the lyopholization product in dichloromethane (5 mL) and adding 2.2 equivalents of 4N HCl in dioxane to afford the title composition as a colorless solid, 154 mg (26%).

MS (ESP): 477 (MH⁺) for C₂₅H₂₅FN₆O₃

¹H-NMR (DMSO-d₆) δ: 1.61 (m, 2H); 2.20 (m, 2H); 2.52 (m, 4H); 3.04 (m, 2H); 3.72 (s, 2H); 4.34 (m, 2H); 4.59 (s, 2H); 4.82 (m, 1H); 6.67 (d, 1H); 7.01 (d, 1H); 7.28 (d, 1H); 7.64 (d, 1H); 7.89 (d, 1H); 8.00 (d, 1H); 8.09 (s, 1H); 11.19 (s, 1H).

### Example 51

### 1-[2-((3S,4R)-3-Methoxy-4-{[(3-oxo-3,4-dihydro-2H-pyrido[3,2-b][1,4]oxazin-6-yl)methyl]amino}piperidin-1-yl)ethyl]-4-methyl-2-oxo-1,2-dihydroquinoline-7-carbonitrile, bis-trifluoroacetic acid salt

1-{2-[(3S,4R)-4-Amino-3-methoxypiperidin-1-yl]ethyl}-4-methyl-2-oxo-1,2-dihydroquinoline-7-carbonitrile, trifluoroacetate, (Intermediate 104, ˜0.52 mmol) was suspended in chloroform/methanol (1:2, 15 mL) and neutralized by dropwise addition of N,N-diisopropylethylamine. 3-Oxo-3,4-dihydro-2H-pyrido[3,2-b][1,4]oxazine-6-carbaldehyde (WO 2004/058144) (93 mg, 0.52 mmol) was added and the reaction was refluxed over 3 Å molecular sieves overnight. Sodium triacetoxyborohydride (138 mg, 0.65 mmol) was added at 0° C. The reaction mixture was stirred at room temperature for 5 hours then filtered through a fritted funnel and concentrated. The residue was taken up in dichloromethane and washed with saturated sodium bicarbonate and saturated sodium chloride. The organic phase was dried over magnesium sulfate and concentrated. Reverse phase chromatography with water/acetonitrile/trifluoroacetic acid afforded title composition as a white solid after lyophilization, 108 mg (34%).

MS (ESP): 503 (MH⁺) for C₂₇H₃₀N₆O₄

¹H-NMR (D₂O-d₂) δ: 2.26 (m, 2H); 2.49 (s, 3H); 3.20 (m, 2H); 3.48 (s, 3H); 3.64 (m, 4H); 4.18 (m, 4H); 4.70 (m, 6H); 6.75 (s, 1H); 7.06 (d, 1H); 7.35 (d, 1H); 7.69 (m, 1H); 7.94 (s, 1H); 8.01 (d, 1H).

### Example 52

### 1-[2-((3R,4S)-3-fluoro-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile, bis hydrochloride salt

1-{2-[(3R,4S)-4-amino-3-fluoropiperidin-1-yl]ethyl}-2-oxo-1,2-dihydroquinoline-7-carbonitrile (Intermediate 106, 352 mg, 1.12 mmol) was suspended in chloroform/methanol (1:1, 30 mL). 3-Oxo-3,4-dihydro-2H-pyrido[3,2-b][1,4]oxazine-6-carbaldehyde (WO 2004/058144) (299 mg, 1.68 mmol) was added and the reaction was refluxed over 3 Å molecular sieves overnight. Sodium triacetoxyborohydride (356 mg, 1.68 mmol) was added at 0° C. The reaction mixture was stirred at room temperature for 5 hours then filtered through a fritted funnel and concentrated. The residue was taken up in dichloromethane and washed with saturated sodium bicarbonate and brine. The organic phase was dried over magnesium sulfate and concentrated. Reverse phase chromatography with water/acetonitrile/ammonium acetate afforded the acetic acid salt of the title compound as an off-white solid after lyophilization. The bis-HCl salt was prepared from a solution of the acetic acid salt in dichloromethane (5 mL), by adding 2.2 equivalents of 4N HCl in dioxane. Filtration and drying under vacuum afforded the desired compound as a colorless solid, 154 mg (26%).

MS (ESP): 477 (MH⁺) for C₂₅H₂₅FN₆O₃

¹H-NMR (DMSO-d₆) δ: 1.57 (m, 2H); 2.37 (m, 2H); 2.58 (m, 4H); 2.91 (m, 2H); 3.71 (s, 2H); 4.34 (m, 2H); 4.59 (s, 2H); 4.70 (m, 1H); 6.76 (d, 1H); 7.00 (d, 1H); 7.28 (d, 1H); 7.63 (d, 1H); 7.89 (d, 1H); 7.98 (d, 1H); 8.08 (s, 1H).

1. A compound of Formula (I): 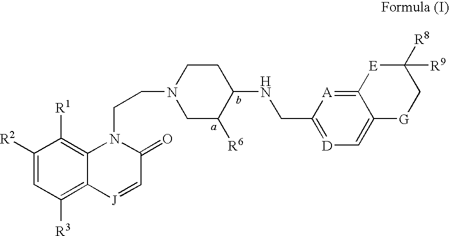
or a pharmaceutically acceptable salt thereof, wherein the compound of Formula (I) is substantially free of a cis (±) mixture of its enantiomers, and wherein
A is selected from CH and N;
D is selected from C—R⁷ and N;
wherein at least one of A and D is carbon;
E is selected from O, NH, and S,
wherein:
i) E is NH if R⁹ and R⁹ together from ═O; and
ii) E is O or S if R⁸ and R⁹ are each H;
G is selected from O and S;
J is selected from C—R⁴ and N;
R¹ is selected from H, halo, cyano, C₁₋₆alkyl, C₂₋₆alkenyl, and C₂₋₆alkynyl, OR^{1a}, and —N(R^{1a})₂, wherein said C₁₋₆alkyl, C₂₋₆alkenyl, and C₂₋₆alkynyl are optionally substituted with one or more R¹⁰;
R^{1a} in each occurrence is independently selected from H and C₁₋₆alkyl, wherein said C₁₋₆alkyl is optionally substituted with one or more R²⁰;
R² is selected from halo, cyano, C₁₋₆alkyl, C₂₋₆alkenyl, C₂₋₆alkynyl, —OR^{2a}, and —N(R^{2a)}₂ wherein said C₁₋₆alkyl, C₂₋₆alkenyl, and C₂₋₆alkynyl are optionally substituted with one or more R²⁰;
R^{2a} in each occurrence is independently selected from H and C₁₋₆alkyl, wherein said C₁₋₆alkyl are optionally substituted with one or more R²⁰;
R³ is selected from H, halo, cyano, C₁₋₆alkyl, C₂₋₆alkenyl, and C₂₋₆alkynyl, —OR^{3a}, and —N(R^{3a})₂, wherein said C₁₋₆alkyl, C₂₋₆alkenyl, and C₂₋₆alkynyl are optionally substituted with one or more R³⁰;
R^{3a} in each occurrence is independently selected from H and C₁₋₆alkyl, wherein said C₁₋₆alkyl is optionally substituted with one or more R³⁰;
R⁴ is selected from H, halo, cyano, —CO₂H, C₁₋₆alkyl, C₂₋₆alkenyl, and C₂₋₆alkynyl, wherein said C₁₋₆alkyl, C₂₋₆alkenyl, and C₂₋₆alkynyl are optionally substituted with one or more R⁴⁰;
R⁶ is selected from fluoro, C₁₋₆alkyl, C₂₋₆alkenyl, C₂₋₆alkynyl, —OR^{6a}, wherein said C₂₋₆alkenyl, and C₂₋₆alkynyl are optionally substituted with one or more R⁶⁰;
R^{6a} in each occurrence is independently selected from H and C₁₋₆alkyl, wherein said C₁₋₆alkyl are optionally substituted with one or more R⁶⁰;
R⁷ is selected from H, halo, cyano, C₁₋₆alkyl, C₂₋₆alkenyl, and C₂₋₆alkynyl, wherein said C₁₋₆alkyl, C₂₋₆alkenyl, and C₂₋₆alkynyl are optionally substituted with one or more R⁷⁰;
R⁸ and R⁹ are each hydrogen, or R⁸ and R⁹ together form ═O; and
R¹⁰, R²⁰, R³⁰, R⁴⁰, R⁶⁰, and R⁷⁰ in each occurrence are each, independently, selected from halo, hydroxy, cyano, —CO₂H, C₁₋₆alkyl, C₂₋₆alkenyl, and C₂₋₆alkynyl.

2. The compound of Formula (I), or a pharmaceutically acceptable salt thereof, as claimed in claim 1, wherein R¹ is H.

3. The compound of Formula (I), or a pharmaceutically acceptable salt thereof, as claimed in claim 1, wherein:
R² is selected from cyano and —OR^{2a}; and
R^{2a} is selected from C₁₋₆alkyl.

4. The compound of Formula (I), or a pharmaceutically acceptable salt thereof, as claimed in claim 1, wherein R³ is H.

5. The compound of Formula (I), or a pharmaceutically acceptable salt thereof, as claimed in claim 1, wherein:
R⁶ is selected from fluoro and —OR^{6a}; and
R^{6a} is selected from H and C₁₋₆alkyl.

6. The compound of Formula (I), or a pharmaceutically acceptable salt thereof, as claimed in claim 1, wherein:
J is selected from C—R⁴ and N; and
R⁴ is selected from H and C₁₋₆alkyl.

7. The compound of Formula (I), or a pharmaceutically acceptable salt thereof, as claimed in claim 1, wherein A, D, E, G, R⁸, and R⁹, together with the ring atoms to which they are attached, form a group selected from:

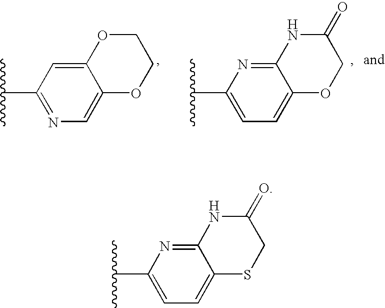

8. The compound of Formula (I), or a pharmaceutically acceptable salt thereof, as claimed in claim 1, wherein
J is selected from N and C—R⁴;
A, D, E, G, R⁸, and R⁹, together with the ring atoms to which they are attached, form a group selected from: 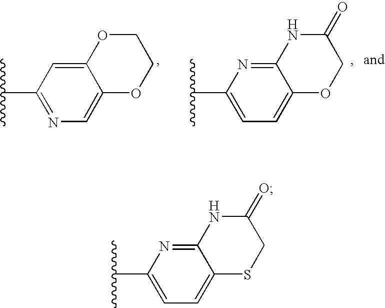
R¹ is H;
R² is selected from cyano and methoxy;
R³ is H;
R⁴ is selected from H and methyl; and
R⁶ is selected from fluoro, hydroxy, and methoxy.

9. The compound of Formula (I), or a pharmaceutically acceptable salt thereof, as claimed in claim 1, wherein the R⁶ group on carbon “a” and the —NH— group on carbon “b” of the compounds of Formula (I) are in a cis (+) relationship to each other.

10. The compound of Formula (I), or a pharmaceutically acceptable salt thereof, as claimed in claim 1, wherein the R⁶ group on carbon “a” and the —NH— group on carbon “b” of the compounds of Formula (I) are in a cis (−) relationship to each other.

11. The compound of Formula (I), or a pharmaceutically acceptable salt thereof, as claimed in claim 1, selected from:
1-[2-((3R,4S)-3-Hydroxy-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile, bis hydrochloride salt;
6-[({(3R,4S)-3-Hydroxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one, bis hydrochloride salt;
1-(2-{(3R,4S)-4-[(2,3-Dihydro[1,4]dioxino[2,3-c]pyridin-7-ylmethyl)amino]-3-fluoropiperidin-1-yl}ethyl)-2-oxo-1,2-dihydroquinoline-7-carbonitrile, monoacetic acid salt;
1-(2-{(3S,4R)-4-[(2,3-Dihydro[1,4]dioxino[2,3-c]pyridin-7-ylmethyl)amino]-3-methoxypiperidin-1-yl}ethyl)-2-oxo-1,2-dihydroquinoline-7-carbonitrile, bis-hydrochloride salt;
1-(2-{(3R,4R)-4-[(2,3-Dihydro[1,4]dioxino[2,3-c]pyridin-7-ylmethyl)amino]-3-methoxypiperidin-1-yl}ethyl)-7-methoxyquinoxalin-2(1H)-one, bis-hydrochloride salt;
1-[2-((3-Fluoro-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile, trans enantiomer A;
1-[2-((3R,4R)-3-methoxy-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile;
1-[2-((3S,4R)-3-Fluoro-4-{[(3-oxo-3,4-dihydro-2H-pyrido[3,2-b][1,4]oxazin-6-yl)methyl]amino}piperidin-1-yl)ethyl]-4-methyl-2-oxo-1,2-dihydroquinoline-7-carbonitrile, bis hydrochloride salt; and
1-[2-((3R,4S)-3-fluoro-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile, bis hydrochloride salt.

12. A process for preparing a compound of Formula (I), or a pharmaceutically acceptable salt thereof, as claimed in claim 1, said process comprising reacting a compound of Formula (AA): 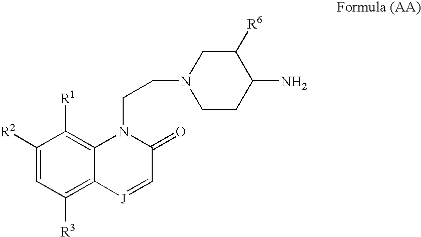
with a compound of Formula (AB): 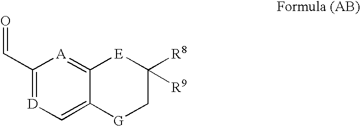
in the presence of a suitable reducing agent,
and thereafter if necessary:
i. converting the compound of Formula (I) into another compound of Formula (I);
ii. removing any protecting groups; and/or
iii. forming a pharmaceutically acceptable salt.

13. A process for preparing a compound of Formula (I), or a pharmaceutically acceptable salt thereof, as claimed in claim 1, said process comprising reacting a compound of Formula (BI): 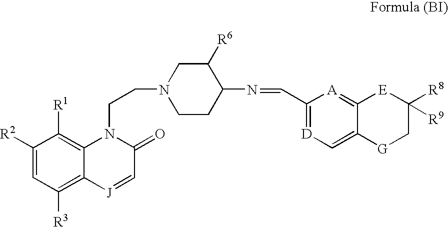
with a suitable reducing agent,
and thereafter if necessary:
i. converting the compound of Formula (I) into another compound of Formula (I);
ii. removing any protecting groups; and/or
iii. forming a pharmaceutically acceptable salt.

14. The use of a compound of Formula (I), or a pharmaceutically acceptable salt thereof, as claimed in claim 1, for the manufacture of a medicament for the treatment of a bacterial infection in a warm-blooded animal such as man.

15. A method for treating a bacterial infection in a warm-blooded animal such as man, said method comprising administering to said animal an effective amount of a compound of Formula (I), or a pharmaceutically acceptable salt thereof, as claimed in claim 1.

16. A compound of Formula (I), or a pharmaceutically acceptable salt thereof, as claimed in claim 1, for use in treating a bacterial infection in a warm-blooded animal such as man.

17. A pharmaceutical composition comprising a compound of Formula (I), or a pharmaceutically acceptable salt thereof, as claimed in claim 1, and at least one pharmaceutically acceptable carrier, diluent, or excipient.

18. A compound of Formula (II): 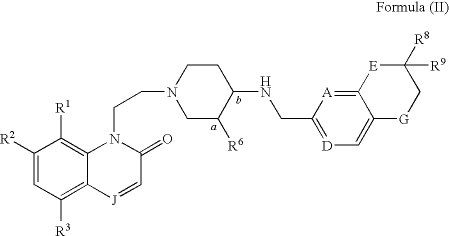
or a pharmaceutically acceptable salt thereof, wherein the R⁶ group on carbon “a” and the —NH— group on carbon “b” are in a trans relationship to each other, and wherein
A is selected from CH and N;
D is selected from C—R⁷ and N;
wherein at least one of A and D is carbon;
E is selected from O, NH, and S,
wherein:
i. E is NH if R⁸ and R⁹ together from ═O; and
ii. E is O or S if R⁸ and R⁹ are each H;
G is selected from O and S;
J is selected from C—R⁴ and N;
R¹ is selected from H, halo, cyano, C₁₋₆alkyl, C₂₋₆alkenyl, and C₂₋₆alkynyl, OR^{1a}, and —N(R^{1a})₂, wherein said C₁₋₆alkyl, C₂₋₆alkenyl, and C₂₋₆alkynyl are optionally substituted with one or more R¹⁰;
R^{1a} in each occurrence is independently selected from H and C₁₋₆alkyl, wherein said C₁₋₆alkyl is optionally substituted with one or more R²⁰;
R² is selected from H, halo, cyano, C₁₋₆alkyl, C₂₋₆alkenyl, C₂₋₆alkynyl, —OR^{2a}, and —N(R^{2a})₂ wherein said C₁₋₆alkyl, C₂₋₆alkenyl, and C₂₋₆alkynyl are optionally substituted with one or more R²⁰;
R^{2a} in each occurrence is independently selected from H and C₁₋₆alkyl, wherein said C₁₋₆alkyl are optionally substituted with one or more R²⁰;
R³ is selected from H, halo, cyano, C₁₋₆alkyl, C₂₋₆alkenyl, and C₂₋₆alkynyl, —OR^{3a}, and —N(R^{3a})₂, wherein said C₁₋₆alkyl, C₂₋₆alkenyl, and C₂₋₆alkynyl are optionally substituted with one or more R³⁰;
R^{3a} in each occurrence is independently selected from H and C₁₋₆alkyl, wherein said C₁₋₆alkyl is optionally substituted with one or more R³⁰;
R⁴ is selected from H, halo, —CO₂H, cyano, C₁₋₆alkyl, C₂₋₆alkenyl, and C₂₋₆alkynyl, wherein said C₁₋₆alkyl, C₂₋₆alkenyl, and C₂₋₆alkynyl are optionally substituted with one or more R⁴⁰;
R⁶ is selected from fluoro, C₁₋₆alkyl, C₂₋₆alkenyl, C₂₋₆alkynyl, —OR^{6a}, wherein said C₁₋₆alkyl, C₂₋₆alkenyl, and C₂₋₆alkynyl are optionally substituted with one or more R⁶⁰;
R^{5a} in each occurrence is independently selected from H and C₁₋₆alkyl, wherein said C₁₋₆alkyl are optionally substituted with one or more R⁶⁰;
R⁷ is selected from H, halo, cyano, C₁₋₆alkyl, C₂₋₆alkenyl, and C₂₋₆alkynyl, wherein said C₁₋₆alkyl, C₂₋₆alkenyl, and C₂₋₆alkynyl are optionally substituted with one or more R⁷⁰;
R⁸ and R⁹ are each hydrogen, or R⁸ and R⁹ together form ═O; and
R¹⁰, R²⁰, R³⁰, R⁴⁰, R⁶⁰, and R⁷⁰ in each occurrence are each, independently, selected from halo, hydroxy, cyano, —CO₂H, C₁₋₆alkyl, C₂₋₆alkenyl, and C₂₋₆alkynyl.

19. The compound of Formula (II), or a pharmaceutically acceptable salt thereof, as claimed in claim 18, wherein R¹ is H.

20. The compound of Formula (II), or a pharmaceutically acceptable salt thereof, as claimed in claim 18, wherein:
R² is selected from cyano and —OR^{2a}; and
R^{2a} is selected from C₁₋₆alkyl.

21. The compound of Formula (II), or a pharmaceutically acceptable salt thereof, as claimed in claim 18, wherein R³ is H.

22. The compound of Formula (II), or a pharmaceutically acceptable salt thereof, as claimed in claim 18, wherein:
R⁶ is selected from fluoro and —OR^{6a}; and
R^{6a} is selected from H and C₁₋₆alkyl.

23. The compound of Formula (II), or a pharmaceutically acceptable salt thereof, as claimed in claim 18, wherein:
J is selected from C—R⁴ and N; and
R⁴ is selected from H and C₁₋₆alkyl.

24. The compound of Formula (II), or a pharmaceutically acceptable salt thereof, as claimed in claim 18, wherein A, D, E, G, R⁸, and R⁹, together with the ring atoms to which they are attached, form a group selected from:

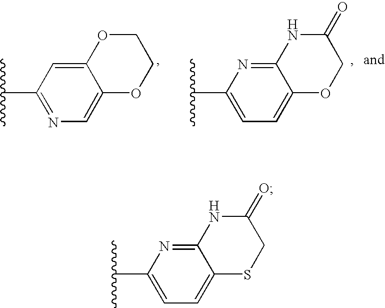

25. The compound of Formula (II), or a pharmaceutically acceptable salt thereof, as claimed in claim 18, wherein:
J is selected from C—R⁴ and N;
A, D, E, G, R⁸, and R⁹, together with the ring atoms to which they are attached, form a group selected from: 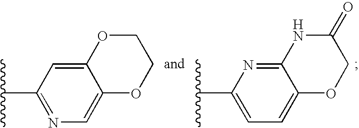
R¹ is H;
R² is selected from cyano and —OR^{2a};
R^{2a} is selected from methyl;
R³ is H; and
R⁴ is selected from H and methyl.

26. The compound of Formula (I), or a pharmaceutically acceptable salt thereof, as claimed in claim 18, selected from:
1-(2-{(3S,4S)-4-[(2,3-Dihydro[1,4]dioxino[2,3-c]pyridin-7-ylmethyl)amino]-3-methoxypiperidin-1-yl}ethyl)-7-methoxyquinoxalin-2(1H)-one, bis-hydrochloride salt;
6-[({(3S,4S)-3-Methoxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one;
1-(2-{(3R,4R)-4-[(2,3-Dihydro[1,4]dioxino[2,3-c]pyridin-7-ylmethyl)amino]3-methoxypiperidin-1-yl}ethyl)-7-methoxyquinoxalin-2(1H)-one, bis-hydrochloride salt;
6-[({(3R,4R)-3-Methoxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one;
1-(2-{4-[(2,3-Dihydro[1,4]dioxino[2,3-c]pyridin-7-ylmethyl)amino]-3-hydroxypiperidin-1-yl}ethyl)-7-methoxyquinoxalin-2(1H)-one, trans enantiomer A, bis hydrochloride salt;
6-[({3-Hydroxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one, trans enantiomer A;
1-(2-{4-[(2,3-dihydro[1,4]dioxino[2,3-c]pyridin-7-ylmethyl)amino]-3-hydroxypiperidin-1-yl}ethyl)-7-methoxyquinoxalin-2(1H)-one, trans enantiomer B, bis-hydrochloride salt;
6-[({3-Hydroxy-1-[2-(7-methoxy-2-oxoquinoxalin-1(2H)-yl)ethyl]piperidin-4-yl}amino)methyl]-2H-pyrido[3,2-b][1,4]oxazin-3(4H)-one, trans enantiomer B;
1-(2-{4-[(2,3-Dihydro[1,4]dioxino[2,3-c]pyridin-7-ylmethyl)amino]-3-fluoropiperidin-1-yl}ethyl)-2-oxo-1,2-dihydroquinoline-7-carbonitrile, trans enantiomer A, bis-hydrochloride salt;
1-[2-((3-Fluoro-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile, trans enantiomer A;
1-(2-{4-[(2,3-Dihydro[1,4]dioxino[2,3-c]pyridin-7-ylmethyl)amino]-3-fluoropiperin-1-yl}ethyl)-7-methoxyquinoxalin-2(1H)-one, trans enantiomer A, bis hydrochloride salt;
6-[({3-Fluoro-1-[2-(7-methoxy-2-oxoquinoxalin-1(2H)-yl)ethyl]piperidin-4-yl}amino)methyl]-2H-pyrido[3,2-b][1,4]oxazin-3(4H)-one, trans enantiomer A;
1-(2-{4-[(2,3-Dihydro[1,4]dioxino[2,3-c]pyridin-7-ylmethyl)amino]-3-fluoropiperin-1-yl}ethyl)-2-oxo-1,2-dihydroquinoline-7-carbonitrile, trans enantiomer B, bis-hydrochloride salt;
1-[2-(3-Fluoro-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile, trans enantiomer B;
1-(2-{4-[(2,3-Dihydro[1,4]dioxino[2,3-c]pyridin-7-ylmethyl)amino]-3-fluoropiperin-1-yl}ethyl)-7-methoxyquinoxalin-2(1H)-one, trans enantiomer B, bis-hydrochloride salt
6-[({3-Fluoro-1-[2-(7-methoxy-2-oxoquinoxalin-1(2H)-yl)ethyl]piperidin-4-yl}amino)methyl]-2H-pyrido[3,2-b][1,4]oxazin-3(4H)-one, trans enantiomer B;
1-(2-{(3R,4R)-4-[(2,3-dihydro[1,4]dioxino[2,3-c]pyridin-7-ylmethyl)amino]-3-methoxypiperidin-1-yl}ethyl)-2-oxo-1,2-dihydroquinoline-7-carbonitrile, bis hydrochloride salt;
1-[2-((3R,4R)-3-methoxy-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile;
1-(2-{(3S,4S)-4-[(2,3-Dihydro[1,4]dioxino[2,3-c]pyridin-7-ylmethyl)amino]-3-methoxypiperidin-1-yl}ethyl)-2-oxo-1,2-dihydroquinoline-7-carbonitrile, bis hydrochloride salt; and
1-[2-((3S,4S)-3-Methoxy-4-{[(3-oxo-3,4-dihydro-2H-pyrido[3,2-b][1,4]oxazin-6-yl)methyl]amino}piperidin-1-yl)ethyl]-2-oxo-1,2-dihydroquinoline-7-carbonitrile.

27. A process for preparing a compound of Formula (II), or a pharmaceutically acceptable salt thereof, as claimed in claim 18, said process comprising reacting a compound of Formula (AA): 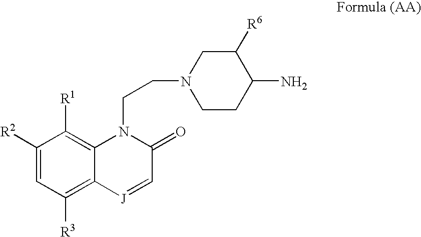
with a compound of Formula (AB): 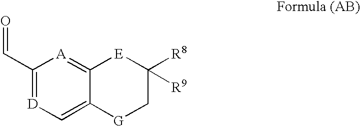
in the presence of a suitable reducing agent,
and thereafter if necessary:
i. converting the compound of Formula (II) into another compound of Formula (II),
ii. removing any protecting groups; and/or
iii. forming a pharmaceutically acceptable salt.

28. A process for preparing a compound of Formula (I), or a pharmaceutically acceptable salt thereof, as claimed in claim 18, said process comprising reacting a compound of Formula (BI): 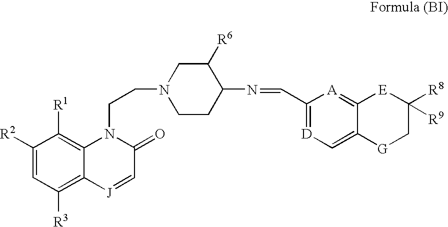
with a suitable reducing agent,
and thereafter if necessary:
i. converting the compound of Formula (II) into another compound of Formula (II),
ii. removing any protecting groups; and/or
iii. forming a pharmaceutically acceptable salt.

29. The use of a compound of Formula (II), or a pharmaceutically acceptable salt thereof, as claimed in claim 18, for the manufacture of a medicament for the treatment of a bacterial infection in a warm-blooded animal such as man.

30. A method for treating a bacterial infection in a warm-blooded animal such as man, said method comprising administering to said animal an effective amount of a compound of Formula (II), or a pharmaceutically acceptable salt thereof, as claimed in claim 18.

31. A compound of Formula (II), or a pharmaceutically acceptable salt thereof, as claimed in claim 18, for use in treating a bacterial infection in a warm-blooded animal such as man.

32. A pharmaceutical composition comprising a compound of Formula (II), or a pharmaceutically acceptable salt thereof, as claimed in claim 18, and at least one pharmaceutically acceptable carrier, diluent, or excipient.
